(19) United States
(12) Patent Application Publication
(10) Pub. No.: US 2008/0175908 A1
(43) Pub. Date: Jul. 24, 2008

(54) TABLET-IN-TABLET COMPOSITIONS

(75) Inventors: Xiuying Liu, Glen Rock, NJ (US); John Kresevic, New Windsor, NY (US); Nizamuddin Baksh, South Hackensack, NJ (US); Robin Enever, New City, NY (US)

Correspondence Address: WilmerHale/Wyeth, 60 STATE STREET, BOSTON, MA, 02109, US

(73) Assignee: Wyeth, Madison, NJ (US)

(21) Appl. No.: 12/013,109

(22) Filed: Jan. 11, 2008

Related U.S. Application Data
(60) Provisional application No. 60/884,801, filed on Jan. 12, 2007.

(51) Int. Cl. A61K 9/28; A61K 31/565

(52) U.S. Cl. 424/465; 514/182; 514/170

(57) ABSTRACT

The present invention is directed to tablet-in-tablet compositions comprising one or more estrogens in a first layer and a therapeutic agent in a second layer, and processes for their preparation

### CROSS-REFERENCE TO RELATED APPLICATIONS

This application claims priority to provisional U.S. Application Ser. No. 60/884,801, filed on Jan. 12, 2007, which is herein incorporated by reference in its entirety.

### FIELD OF THE INVENTION

The invention is directed generally to the field of pharmaceutical formulations. More specifically, the invention relates to tablet-in-tablet compositions and methods of preparing such compositions. In some embodiments, the compositions comprise one or more estrogens in a core tablet and one or more therapeutic agents in a compressed outer tablet layer.

### BACKGROUND OF THE INVENTION

Menopause is generally defined as the last natural menstrual period and is characterized by the cessation of ovarian function, leading to the substantial diminution of circulating estrogen in the bloodstream. Menopause is usually identified, in retrospect, after 12 months of amenorrhea. It is usually not a sudden event, but is often preceded by a time of irregular menstrual cycles prior to eventual cessation of menses. Following the cessation of menstruation, the decline in endogenous estrogen concentrations is typically rapid. There is a decrease in serum estrogens from circulating levels ranging from 40-250 pg/mL of estradiol and 40-170 pg/mL of estrone during ovulatory cycles to less than 15 pg/mL of estradiol and 30 pg/mL of estrone in postmenopausal women.

As these estrogens decline during the time preceding (perimenopause) and following the menopause (postmenopause), various physiological changes may result, including vulvar and vaginal atrophy causing vaginal dryness, pruritus and dyspareunia, and vasomotor instability manifested as hot flushes. Other menopausal disturbances may include depression, insomnia, and nervousness. The long-term physiologic effects of postmenopausal estrogen deprivation may result in significant morbidity and mortality due to increase in the risk factors for cardiovascular disease and osteoporosis. Menopausal changes in blood lipid levels, a major component of the pathogenesis of coronary heart disease (CHD), may be precursors to increased incidence of ischemic heart disease, atherosclerosis, and other cardiovascular disease. A rapid decrease in bone mass of both cortical (spine) and trabecular (hip) bone can be seen immediately after the menopause.

Estrogen replacement therapy (ERT) is beneficial for symptomatic relief of hot flushes and genital atrophy and for prevention of postmenopausal osteoporosis. ERT has been recognized as an advantageous treatment for relief of vasomotor symptoms. Long term ERT can prevent osteoporosis because it decreases bone loss, reduces spine and hip fracture, and prevents loss of height. In addition, ERT has been shown to be effective in increasing high density lipoprotein-cholesterol (HDL-C) and in reducing low density lipoprotein cholesterol (LDL-C), affording possible protection against CHD. ERT also can provide antioxidant protection against free radical mediated disorders or disease states. Estrogens have also been reported to confer neuroprotection, and inhibit neurodegenerative disorders, such as Alzheimer's disease (see U.S. Pat. No. 5,554,601, which is hereby incorporated by reference in its entirety).

The normal protocol for ERT calls for estrogen supplementation using such formulations containing estrone, estriol, ethynyl estradiol or conjugated estrogens isolated from natural sources (i.e. PREMARIN® conjugated estrogens from Wyeth, Madison, N.J.). In some patients, therapy may be contraindicated due to the proliferative effects of unopposed estrogens have on uterine tissue. This proliferation is associated with increased risk for endometriosis and/or endometrial cancer. The effects of unopposed estrogens on breast tissue are less clear, but are of some concern. Accordingly, one trend has been towards the development of low dose treatment regimens that minimize the adverse effects of ERT.

Another approach has been to administer a progestin, either sequentially or in combination, with the estrogen. There are extensive clinical data showing that the relative risk of endometrial cancer can be reduced by the addition of a progestin to ERT. The addition of a progestin to estrogen therapy can help prevent estrogen-induced endometrial proliferation. With appropriate doses of daily estrogen and progestin, combined estrogen replacement therapy has been shown to be effective in relieving vaginal atrophy and vasomotor symptoms, preventing postmenopausal osteoporosis, and reducing the risk of endometrial cancer by prevention of endometrial hyperplasia.

A third approach to minimize the adverse effects of ERT is the use of selective estrogen receptor modulators (SERMs) in conjunction with ERT. Selective SERMs are a class of compounds that demonstrate an affinity for estrogen receptors (ER) but show tissue selective estrogenic effects. An example of a SERM is bazedoxifene acetate (1-[4-(2-azepan-1-yl-ethoxy)-benzyl]-2-(4-hydroxy-phenyl)-3-methyl-1H-indol-5-ol acetic acid), having the chemical formula shown below:

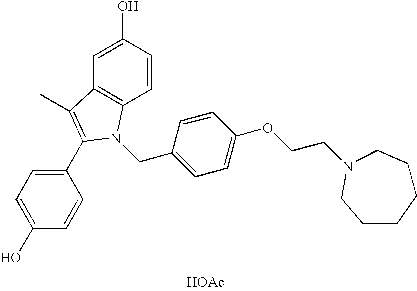

Bazedoxifene acetate (“BZA”) has been reported to prevent bone loss and protect the cardiovascular system and reduce or eliminate the negative effects on the uterus and breast (potential risk of uterine and breast cancers). Consistent with its classification as a SERM, bazedoxifene acetate demonstrates little or no stimulation of uterine response in preclinical models of uterine stimulation. Conversely, bazedoxifene acetate demonstrates an estrogen agonist-like effect in preventing bone loss and reducing cholesterol in an ovariectomized rat model of osteopenia. In an MCF-7 cell line (human breast cancer cell line), bazedoxifene acetate behaves as an estrogen antagonist. These data demonstrate that bazedoxifene acetate is estrogenic on bone and cardiovascular lipid parameters and antiestrogenic on uterine and mammary tissue and thus has the potential for treating a number of different diseases or disease-like states wherein the estrogen receptor is involved.

In summary, a number of different approaches exist for minimizing the adverse side-effects of ERT, including the administration of a progestin or SERM in conjunction with ERT. Given the growing trend towards progestin/estrogen and SERM/estrogen therapies, there is an interest in developing a single dosage form which can deliver multiple drugs at different release rates. There is a particular need to develop drug delivery systems which can be tailored to give any desired treatment regimen, e.g., fast release of one drug and sustained release of another, sustained release of both drugs, sequential dosing regimens, continuous dosing regimens and the like, in order to improve clinical outcome. Additionally, there is a need to improve patient compliance by eliminating the need for separate administration of multiple drugs. This invention meets these needs and others.

### SUMMARY OF THE INVENTION

In a first aspect, the present invention provides tablet-in-tablet compositions comprising:

a) a core tablet comprising:
one or more estrogens;
a core filler/diluent component comprising from about 30% to about 85% by weight of the core tablet;
a core filler/binder component comprising from about 1% to about 30% by weight of the core tablet;
a core hydrophilic gel-forming polymer component comprising from about 1% to about 40% by weight of the core tablet; and
optionally, a core lubricant component comprising from about 0.01% to about 2% by weight of the core tablet; and

b) a compressed outer tablet layer comprising:
one or more therapeutic agents selected from the group consisting of selective estrogen receptor modulators and progestational agents;
an outer layer filler/diluent component comprising from about 10% to about 80% by weight of the compressed outer tablet layer;
an outer layer filler/binder component comprising from about 1% to about 70% by weight of the compressed outer tablet layer;
an outer layer hydrophilic gel-forming polymer component comprising from about 1% to about 70% by weight of the compressed outer tablet layer;
optionally, an antioxidant component comprising from about 0.01% to about 4% by weight of the compressed outer tablet layer; and
optionally, an outer layer lubricant component comprising from about 0.01% to about 2% by weight of the compressed outer tablet layer.

In a second aspect, the present invention provides tablet-in-tablet compositions comprising:

a) a core tablet comprising:
one or more estrogens;
a core filler/diluent component comprising from about 30% to about 85% by weight by weight of the core tablet;
a core filler/binder component comprising from about 1% to about 30% by weight of the core tablet;
a core hydrophilic gel-forming polymer component comprising from about 1% to about 40% by weight of the core tablet; and
optionally, a core lubricant component comprising from about 0.01% to about 2% by weight of the core tablet; and

b) a compressed outer tablet layer comprising:
one or more therapeutic agents selected from the group consisting of selective estrogen receptor modulators and progestational agents;
a pharmaceutically acceptable carrier component comprising from about 60% to about 99.9% by weight of the compressed outer tablet layer, wherein the pharmaceutically acceptable carrier component optionally comprises one or more of an outer layer filler/diluent component, an outer layer filler/binder component, and an outer layer hydrophilic gel-forming polymer component;
optionally, an outer layer lubricant component comprising from about 0.01% to about 2% by weight of the compressed outer tablet layer; and
optionally, an antioxidant component comprising from about 0.01% to about 4% by weight of the compressed outer tablet layer.

In a third aspect, the present invention provides tablet-in-tablet compositions comprising:

a) a core tablet comprising:
one or more estrogens;
a core filler/diluent component comprising from about 30% to about 85% by weight by weight of the core tablet;
a core filler/binder component comprising from about 1% to about 30% by weight of the core tablet;
a core hydrophilic gel-forming polymer component comprising from about 1% to about 40% by weight of the core tablet; and
optionally, a core lubricant component comprising from about 0.01% to about 2% by weight of the core tablet; and

b) a compressed outer tablet layer comprising:
one or more therapeutic agents selected from the group consisting of selective estrogen receptor modulators and progestational agents;
an outer layer filler/diluent component comprising from about 25% to about 65% by weight of the compressed outer tablet layer;
an outer layer filler/binder component comprising from about 20% to about 50% by weight of the compressed outer tablet layer;
a disintegrant component comprising from about 2% to about 15% by weight of the compressed outer tablet layer;
optionally, an outer layer wetting agent component comprising from about 0.01% to about 4% of the compressed outer tablet layer;
optionally, an outer layer lubricant component comprising from about 0.01% to about 2% by weight of the compressed outer tablet layer; and
optionally, an antioxidant component comprising from about 0.01% to about 4% by weight of the compressed outer tablet layer.

In some embodiments, the present invention further provides a tablet-in-tablet composition selected from a plurality of tablet-in-tablet compositions, wherein the plurality has a content uniformity for the therapeutic agent about equal to or less than 3.5% or 2.5%.

In some embodiments, the present invention further provides a tablet-in-tablet composition selected from a plurality of tablet-in-tablet compositions, wherein the plurality has a weight variation of about equal to or less than 2% or 1.5%.

In some embodiments, the present invention provides tablet-in-tablet compositions selected from a plurality of compositions according to the first aspect of the invention, wherein the plurality has a mean dissolution profile wherein: the mean of % of the estrogen released per composition after 1, 2, 3, 4, and 5 hours under estrogen dissolution conditions is substantially equal to the sum of b₁*X₁, b₂X₂, b₃*X₃, b₁₂*X₁*X₂, b₁₃*X₁*X₃, and b₂₃*X₂*X₃; and

the mean of % of the therapeutic agent per composition released after 0.25, 0.5, 1, 2, and 6 hours under type I therapeutic agent dissolution conditions is substantially equal to the sum of a₁*X₁, b₂X₂, a₃*X₃, a₁₂*X₁*X₂, a₁₃*X₁*X₃, and a₂₃*X₂*X₃;

X₁ is the % by weight of the outer layer hydrophilic gel-forming polymer component in the compressed outer tablet layer;

X₂ is the % by weight of the outer layer filler/diluent component in the compressed outer tablet layer;

X₃ is the % by weight of the outer layer filler/binder component in the compressed outer tablet layer;

b₁ at 1 hour is 157.4;

b₁ at 2 hours is 193.09;

b₁ at 3 hours is 184.1;

b₁ at 4 hours is 146.45;

b₁ at 5 hours is 100.25;

b₂ at 1 hour is 54.47;

b₂ at 2 hours is 80.09;

b₂ at 3 hours is 93.71;

b₂ at 4 hours is 101.05;

b₂ at 5 hours is 104.11;

b₃ at 1 hour is 46.75;

b₃ at 2 hours is 69.86;

b₃ at 3 hours is 84.19;

b₃ at 4 hours is 92.12;

b₃ at 5 hours is 95.89;

b₁₂ at 1 hour is −437.12;

b₁₂ at 2 hours is −557.91;

b₁₂ at 3 hours is −561.48;

b₁₂ at 4 hours is −489.08;

b₁₂ at 5 hours is −383.44;

b₁₃ at 1 hour is −414.17;

b₁₃ at 2 hours is −542.65;

b₁₃ at 3 hours is −569.13;

b₁₃ at 4 hours is −518.63;

b₁₃ at 5 hours is −441.05;

b₂₃ at 1 hour is 76.74;

b₂₃ at 2 hours is 79.7;

b₂₃ at 3 hours is 65.43;

b₂₃ at 4 hours is 43.23;

b₂₃ at 5 hours is 29.91;

a₁ at 0.25 hour is 217.8;

a₁ at 0.5 hour is 218.36;

a₁ at 1 hour is 188.75;

a₁ at 2 hours is 121.23;

a₁ at 6 hours is −21.48;

a₂ at 0.25 hour is 87.91;

a₂ at 0.5 hour is 93.12;

a₂ at 1 hour is 96.98;

a₂ at 2 hours is 100.52;

a₂ at 6 hours is 100.91;

a₃ at 0.25 hour is 58.83;

a₃ at 0.5 hour is 75.08;

a₃ at 1 hour is 86.32;

a₃ at 2 hours is 92.04;

a₃ at 6 hours is 99.99;

a₁₂ at 0.25 hour is −616.98;

a₁₂ at 0.5 hour is −617.39;

a₁₂ at 1 hour is −545.68;

a₁₂ at 2 hours is −377.76;

a₁₂ at 6 hours is 69.72;

a₁₃ at 0.25 hour is −536.63;

a₁₃ at 0.5 hour is −576.95;

a₁₃ at 1 hour is −540.35;

a₁₃ at 2 hours is −397.91;

a₁₃ at 6 hours is 12.22;

a₂₃ at 0.25 hour is 30.77;

a₂₃ at 0.5 hour is 31.94;

a₂₃ at 1 hour is 32.68;

a₂₃ at 2 hours is 32.91; and

a₂₃ at 6 hours is 9.65.

In some embodiments, the present invention provides tablet-in-tablet compositions selected from a plurality of compositions according to the second aspect of the invention, wherein the plurality has a mean dissolution profile wherein:

the mean of % of the estrogen released per composition after 1, 2, 3, 4, and 5 hours under estrogen dissolution conditions is substantially equal to the sum of b₁*X₁, b₂X₂, b₃*X₃, b₁₂*X₁*X₂, b₁₃*X₁*X₃, and b₂₃*X₂*X₃;

the mean of % of the therapeutic agent per composition released after 0.25, 0.5, 1, 2, and 6 hours under type I therapeutic agent dissolution conditions is substantially equal to the sum of a₁*X₁, b₂X₂, a₃*X₃, a₁₂*X₁*X₂, a₁₃*X₁*X₃, and a₂₃*X₂*X₃;

X₁ is the % by weight of the optional outer layer hydrophilic gel-forming polymer component, if present, in the compressed outer tablet layer;

X₂ is the % by weight of the optional outer layer filler/diluent component, if present, in the compressed outer tablet layer; and

X₃ is the % by weight of the optional outer layer filler/binder component, if present, in the compressed outer tablet layer;

b₁ at 1 hour is 157.4;

b₁ at 2 hours is 193.09;

b₁ at 3 hours is 184.1;

b₁ at 4 hours is 146.45;

b₁ at 5 hours is 100.25;

b₂ at 1 hour is 54.47;

b₂ at 2 hours is 80.09;

b₂ at 3 hours is 93.71;

b₂ at 4 hours is 101.05;

b₂ at 5 hours is 104.11;

b₃ at 1 hour is 46.75;

b₃ at 2 hours is 69.86;

b₃ at 3 hours is 84.19;

b₃ at 4 hours is 92.12;

b₃ at 5 hours is 95.89;

b₁₂ at 1 hour is −437.12;

b₁₂ at 2 hours is −557.91;

b₁₂ at 3 hours is −561.48;

b₁₂ at 4 hours is −489.08;

b₁₂ at 5 hours is −383.44;

b₁₃ at 1 hour is −414.17;

b₁₃ at 2 hours is −542.65;

b₁₃ at 3 hours is −569.13;

b₁₃ at 4 hours is −518.63;

b₁₃ at 5 hours is −441.05;

b₂₃ at 1 hour is 76.74;

b₂₃ at 2 hours is 79.7;

b₂₃ at 3 hours is 65.43;

b₂₃ at 4 hours is 43.23;

b₂₃ at 5 hours is 29.91;

a₁ at 0.25 hour is 217.8;

a₁ at 0.5 hour is 218.36;

a₁ at 1 hour is 188.75;

a₁ at 2 hours is 121.23;

a₁ at 6 hours is −21.48;

a₂ at 0.25 hour is 87.91;

a₂ at 0.5 hour is 93.12;

a₂ at 1 hour is 96.98;

a₂ at 2 hours is 100.52;

a₂ at 6 hours is 100.91;

a₃ at 0.25 hour is 58.83;

a₃ at 0.5 hour is 75.08;

a₃ at 1 hour is 86.32;

a₃ at 2 hours is 92.04;

a₃ at 6 hours is 99.99;

a₁₂ at 0.25 hour is −616.98;

a₁₂ at 0.5 hour is −617.39;

a₁₂ at 1 hour is −545.68;

a₁₂ at 2 hours is −377.76;

a₁₂ at 6 hours is 69.72;

a₁₃ at 0.25 hour is −536.63;

a₁₃ at 0.5 hour is −576.95;

a₁₃ at 1 hour is −540.35;

a₁₃ at 2 hours is −397.91;

a₁₃ at 6 hours is 12.22;

a₂₃ at 0.25 hour is 30.77;

a₂₃ at 0.5 hour is 31.94;

a₂₃ at 1 hour is 32.68;

a₂₃ at 2 hours is 32.91; and

a₂₃ at 6 hours is 9.65.

In some embodiments, the present invention provides tablet-in-tablet compositions wherein:
the core tablet comprises at least one conjugated estrogen;
the compressed outer tablet layer comprises bazedoxifene acetate;
the dissolution profile of the estrogen from the composition under estrogen dissolution conditions is substantially as shown in any one of FIG. 30 to 32 or 48 to 54; and
the dissolution profile of the therapeutic agent from the composition under type II therapeutic agent dissolution conditions is substantially as shown in any one of FIGS. 27 to 29 or 41 to 47.

In some embodiments, the present invention provides tablet-in-tablet compositions wherein:
the core tablet comprises at least one conjugated estrogen;
the compressed outer tablet layer comprises medroxyprogesterone acetate;
the dissolution profile of the estrogen from the composition under estrogen dissolution conditions is substantially as shown in any one of FIGS. 4-6, FIG. 33 (Example 9), FIG. 34 (Example 13), FIG. 35 (Example 15), FIG. 35 (Example 16), FIG. 35 (Example 18) or FIG. 36 (Example 20); and
the dissolution profile of the therapeutic agent from the composition under type I therapeutic agent dissolution conditions is substantially as shown in any one of FIGS. 1-3, FIG. 37 (Example 9), FIG. 38 (Example 13), FIG. 39 (Example 15), FIG. 39 (Example 16), FIG. 39 (Example 18) or FIG. 40 (Example 20).

In some embodiments, the present invention provides tablet-in-tablet compositions wherein:
the core tablet comprises at least one conjugated estrogen;
the compressed outer tablet layer comprises medroxyprogesterone acetate;
the dissolution profile of the estrogen from the composition under estrogen dissolution conditions is substantially as shown in any one of FIG. 33 (Example 8), FIG. 33 (Example 10), FIG. 33 (Example 11), FIG. 34 (Example 12), FIG. 34 (Example 14), FIG. 35 (Example 17), FIG. 36 (Example 19) or FIG. 36 (Example 21); and
the dissolution profile of the therapeutic agent from the composition under type I therapeutic agent dissolution conditions is substantially as shown in any one of FIG. 37 (Example 8), FIG. 37 (Example 10), FIG. 38 (Example 11), FIG. 38 (Example 12), FIG. 38 (Example 14), FIG. 39 (Example 17), FIG. 40 (Example 19) or FIG. 40 (Example 21).

The present invention also provides processes for producing the tablet-in-tablet compositions of the invention. Accordingly, in one aspect, the present invention provides a process for producing a tablet-in-tablet composition of the invention comprising compressing a first solid mixture to form a core tablet; and

compressing a second solid mixture onto the core tablet to form a compressed outer tablet layer;

wherein:

(a) the first solid mixture comprises:
one or more estrogens;
a first solid mixture filler/diluent component comprising from about 30% to about 85% by weight of the first solid mixture;
a first solid mixture filler/binder component comprising from about 1% to about 30% by weight of the first solid mixture;
a first solid mixture hydrophilic gel-forming polymer component comprising from about 1% to about 40% by weight of the first solid mixture; and
optionally, a first solid mixture lubricant component comprising from about 0.01% to about 2% by weight of the first solid mixture; and

(b) the second solid mixture comprises:
one or more therapeutic agents selected from the group consisting of selective estrogen receptor modulator and a progestational agent;
a second solid mixture filler/diluent component comprising from about 10% to about 80% by weight of the second solid mixture;
a second solid mixture filler/binder component comprising from about 1% to about 70% by weight of the second solid mixture;
a second solid mixture hydrophilic gel-forming polymer component comprising from about 1% to about 60% of the compressed outer tablet layer;
optionally, a second solid mixture antioxidant component comprising from about 0.01% to about 4% of the second solid mixture; and
optionally, a second solid mixture lubricant component comprising from about 0.01% to about 2% of the second solid mixture.

In another aspect, the present invention provides a process for producing a tablet-in-tablet composition comprising:
compressing a first solid mixture to form a core tablet; and
compressing a second solid mixture onto the core tablet to form a compressed outer tablet layer;
   wherein:

a) the first solid mixture comprises:
one or more estrogens;
a first solid mixture filler/diluent component comprising from about 30% to about 85% by weight by weight of the core tablet;
a first solid mixture filler/binder component comprising from about 1% to about 30% by weight of the core tablet;
a first solid mixture hydrophilic gel-forming polymer component comprising from about 1% to about 40% by weight of the core tablet; and
optionally, a first solid mixture lubricant component comprising from about 0.01% to about 2% by weight of the core tablet; and

b) the second solid mixture comprises:
one or more therapeutic agents selected from the group consisting of selective estrogen receptor modulators and progestational agents;
a pharmaceutically acceptable carrier component comprising from about 60% to about 99.9% by weight of the compressed outer tablet layer, wherein the outer pharmaceutically acceptable carrier component optionally comprises one or more of a second solid mixture filler/diluent component, a second solid mixture filler/binder component, and a second solid mixture hydrophilic gel-forming polymer component;
optionally, a second solid mixture lubricant component comprising from about 0.01% to about 2% by weight of the compressed outer tablet layer; and
optionally, a second solid mixture antioxidant component comprising from about 0.01% to about 4% by weight of the compressed outer tablet layer.

In another aspect, the present invention provides a process for producing a tablet-in-tablet composition comprising:
compressing a first solid mixture to form a core tablet; and
compressing a second solid mixture onto the core tablet to form a compressed outer tablet layer;
   wherein:

a) the first solid mixture comprises:
one or more estrogens;
a core filler/diluent component comprising from about 30% to about 85% by weight by weight of the core tablet;
a core filler/binder component comprising from about 1% to about 30% by weight of the core tablet;
a core hydrophilic gel-forming polymer component comprising from about 1% to about 40% by weight of the core tablet; and
optionally, a core lubricant component comprising from about 0.01% to about 2% by weight of the core tablet; and

b) the second solid mixture comprises:
one or more therapeutic agents selected from the group consisting of selective estrogen receptor modulators and progestational agents;
an outer layer filler/diluent component comprising from about 25% to about 65% by weight of the compressed outer tablet layer;
an outer layer filler/binder component comprising from about 20% to about 50% by weight of the compressed outer tablet layer;
a disintegrant component comprising from about 2% to about 15% by weight of the compressed outer tablet layer;
optionally, an outer layer wetting agent component comprising from about 0.01% to about 4% of the compressed outer tablet layer;
optionally, an outer layer lubricant component comprising from about 0.01% to about 2% by weight of the compressed outer tablet layer; and
optionally, an antioxidant component comprising from about 0.01% to about 4% by weight of the compressed outer tablet layer.

In some embodiments, the processes produce a plurality of tablet-in-tablet compositions having a content uniformity for the therapeutic agent about equal to or less than 3.5% or 2.5%.

In some embodiments, the processes produce a plurality of tablet-in-tablet compositions having a weight variation about equal to or less than 2% or 1.5%.

The present invention further provides products produced by the processes of the invention.

The present invention further provides a plurality of products produced by the processes of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### DESCRIPTION OF THE FIGURES

FIG. 1 is a line graph depicting the % of MPA released over time for Example 5 (see Table 20, Example 5 for each data point and the associated standard deviation).

FIG. 2 is a line graph depicting the % of MPA released over time for Example 6 (see Table 20, Example 6 for each data point and the associated standard deviation).

FIG. 3 is a line graph depicting the % of MPA released over time for Example 7 (see Table 20, Example 7 for each data point and the associated standard deviation).

FIG. 4 is a line graph depicting the % of CE released over time for Example 5 (see Table 21, Example 5, for each data point and the associated standard deviation).

FIG. 5 is a line graph depicting the % of CE released over time for Example 6 (see Table 21, Example 6, for each data point and the associated standard deviation).

FIG. 6 is a line graph depicting the % of CE released over time for Example 7 (see Table 21, Example 7, for each data point and the associated standard deviation).

FIG. 7 is a plot depicting the effect of hydroxypropylmethylcellulose (“HPMC”), lactose monohydrate (“lactose”) and microcrystalline cellulose (“AVICEL®”) levels on the % of CE released in 1 hour from the tablet-in-tablet compositions.

FIG. 8 is a line graph depicting the effect of hydroxypropylmethylcellulose (“HPMC”), lactose monohydrate (“lactose”) and microcrystalline cellulose (“AVICEL®”) levels on the % of CE released in 1 hour from the tablet-in-tablet compositions.

FIG. 9 is a plot depicting the effect of hydroxypropylmethylcellulose (“HPMC”), lactose monohydrate (“lactose”) and microcrystalline cellulose (“AVICEL®”) levels on the % of CE released in 2 hours from the tablet-in-tablet compositions.

FIG. 10 is a line graph depicting the effect of hydroxypropylmethylcellulose (“HPMC”), lactose monohydrate (“lactose”) and microcrystalline cellulose (“AVICEL®”) levels on the % of CE released in 2 hours from the tablet-in-tablet compositions.

FIG. 11 is a plot depicting the effect of hydroxypropylmethylcellulose (“HPMC”), lactose monohydrate (“lactose”) and microcrystalline cellulose (“AVICEL®”) levels on the % of CE released in 3 hours from the tablet-in-tablet compositions.

FIG. 12 is a line graph depicting the effect of hydroxypropylmethylcellulose (“HPMC”), lactose monohydrate (“lactose”) and microcrystalline cellulose (“AVICEL®”) levels on the % of CE released in 3 hours from the tablet-in-tablet compositions.

FIG. 13 is a plot depicting the effect of hydroxypropylmethylcellulose (“HPMC”), lactose monohydrate (“lactose”) and microcrystalline cellulose (“AVICEL®”) levels on the % of CE released in 4 hours from the tablet-in-tablet compositions.

FIG. 14 is a line graph depicting the effect of hydroxypropylmethylcellulose (“HPMC”), lactose monohydrate (“lactose”) and microcrystalline cellulose (“AVICEL®”) levels on the % of CE released in 4 hours from the tablet-in-tablet compositions.

FIG. 15 is a plot depicting the effect of hydroxypropylmethylcellulose (“HPMC”), lactose monohydrate (“lactose”) and microcrystalline cellulose (“AVICEL®”) levels on the % of CE released in 5 hours from the tablet-in-tablet compositions.

FIG. 16 is a line graph depicting the effect of hydroxypropylmethylcellulose (“HPMC”), lactose monohydrate (“lactose”) and microcrystalline cellulose (“AVICEL®”) levels on the % of CE released in 5 hours from the tablet-in-tablet compositions.

FIG. 17 is a plot depicting the effect of hydroxypropylmethylcellulose (“HPMC”), lactose monohydrate (“lactose”) and microcrystalline cellulose (“AVICEL®”) levels on the % of MPA released in 15 minutes from the tablet-in-tablet compositions.

FIG. 18 is a line graph depicting the effect of hydroxypropylmethylcellulose (“HPMC”), lactose monohydrate (“lactose”) and microcrystalline cellulose (“AVICEL®”) levels on the % of MPA released in 15 minutes from the tablet-in-tablet compositions.

FIG. 19 is a plot depicting the effect of hydroxypropylmethylcellulose (“HPMC”), lactose monohydrate (“lactose”) and microcrystalline cellulose (“AVICEL®”) levels on the % of MPA released in 30 minutes from the tablet-in-tablet compositions.

FIG. 20 is a line graph depicting the effect of hydroxypropylmethylcellulose (“HPMC”), lactose monohydrate (“lactose”) and microcrystalline cellulose (“AVICEL®”) levels on the % of MPA released in 30 minutes from the tablet-in-tablet compositions.

FIG. 21 is a plot depicting the effect of hydroxypropylmethylcellulose (“HPMC”), lactose monohydrate (“lactose”) and microcrystalline cellulose (“AVICEL®”) levels on the % of MPA released in 60 minutes from the tablet-in-tablet compositions.

FIG. 22 is a line graph depicting the effect of hydroxypropylmethylcellulose (“HPMC”), lactose monohydrate (“lactose”) and microcrystalline cellulose (“AVICEL®”) levels on the % of MPA released in 60 minutes from the tablet-in-tablet compositions.

FIG. 23 is a plot depicting the effect of hydroxypropylmethylcellulose (“HPMC”), lactose monohydrate (“lactose”) and microcrystalline cellulose (“AVICEL®”) levels on the % of MPA released in 120 minutes from the tablet-in-tablet compositions.

FIG. 24 is a line graph depicting the effect of hydroxypropylmethylcellulose (“HPMC”), lactose monohydrate (“lactose”) and microcrystalline cellulose (“AVICEL®”) levels on the % of MPA released in 120 minutes from the tablet-in-tablet compositions.

FIG. 25 is a plot depicting the effect of hydroxypropylmethylcellulose (“HPMC”), lactose monohydrate (“lactose”) and microcrystalline cellulose (“AVICEL®”) levels on the % of MPA released in 360 minutes from the tablet-in-tablet compositions.

FIG. 26 is a line graph depicting the effect of hydroxypropylmethylcellulose (“HPMC”), lactose monohydrate (“lactose”) and microcrystalline cellulose (“AVICEL®”) levels on the % of MPA released in 360 minutes from the tablet-in-tablet compositions.

FIG. 27 is a line graph depicting the % of BZA released over time for Example 34A (see Table 46, Example 34A for each data point and the associated standard deviation).

FIG. 28 is a line graph depicting the % of BZA released over time for Example 34B (see Table 46, Example 34B for each data point and the associated standard deviation).

FIG. 29 is a line graph depicting the % of BZA released over time for Example 34C (see Table 46, Example 34C for each data point and the associated standard deviation).

FIG. 30 is a line graph depicting the % of CE released over time for Example 34A (see Table 47, Example 34A for each data point and the associated standard deviation).

FIG. 31 is a line graph depicting the % of CE released over time for Example 34B (see Table 47, Example 34B for each data point and the associated standard deviation).

FIG. 32 is a line graph depicting the % of CE released over time for Example 34C (see Table 47, Example 34C for each data point and the associated standard deviation).

FIG. 33 is a line graph depicting the % of CE released over time for Examples 8-11 (see Table 23, Examples 8-11 for each data point and the associated standard deviation).

FIG. 34 is a line graph depicting the % of CE released over time for Examples 12-14 (see Table 23, Examples 12-14 for each data point and the associated standard deviation).

FIG. 35 is a line graph depicting the % of CE released over time for Examples 15-18 (see Table 23, Examples 15-18 for each data point and the associated standard deviation).

FIG. 36 is a line graph depicting the % of CE released over time for Examples 19-21 (see Table 23, Examples 19-21 for each data point and the associated standard deviation).

FIG. 37 is a line graph depicting the % of MPA released over time for Examples 8-10 (see Table 22, Examples 8-10 for each data point and the associated standard deviation).

FIG. 38 is a line graph depicting the % of MPA released over time for Examples 11-14 (see Table 22, Examples 11-14 for each data point and the associated standard deviation).

FIG. 39 is a line graph depicting the % of MPA released over time for Examples 15-18 (see Table 22, Examples 15-18 for each data point and the associated standard deviation).

FIG. 40 is a line graph depicting the % of MPA released over time for Examples 19-21 (see Table 22, Examples 19-21 for each data point and the associated standard deviation).

FIG. 41 is a line graph depicting the % of BZA released over time for Example 34D (see Table 48 for each data point and the associated standard deviation).

FIG. 42 is a line graph depicting the % of BZA released over time for Example 34E (see Table 48 for each data point and the associated standard deviation).

FIG. 43 is a line graph depicting the % of BZA released over time for Example 34F (see Table 48 for each data point and the associated standard deviation).

FIG. 44 is a line graph depicting the % of BZA released over time for Example 34G (see Table 48 for each data point and the associated standard deviation).

FIG. 45 is a line graph depicting the % of BZA released over time for Example 34H (see Table 48 for each data point and the associated standard deviation).

FIG. 46 is a line graph depicting the % of BZA released over time for Example 341 (see Table 48 for each data point and the associated standard deviation).

FIG. 47 is a line graph depicting the % of BZA released over time for Example 34J (see Table 48 for each data point and the associated standard deviation).

FIG. 48 is a line graph depicting the % of CE released over time for Example 34D (see Table 49 for each data point and the associated standard deviation).

FIG. 49 is a line graph depicting the % of CE released over time for Example 34E (see Table 49 for each data point and the associated standard deviation).

FIG. 50 is a line graph depicting the % of CE released over time for Example 34F (see Table 49 for each data point and the associated standard deviation).

FIG. 51 is a line graph depicting the % of CE released over time for Example 34G (see Table 49 for each data point and the associated standard deviation).

FIG. 52 is a line graph depicting the % of CE released over time for Example 34H (see Table 49 for each data point and the associated standard deviation).

FIG. 53 is a line graph depicting the % of CE released over time for Example 341 (see Table 49 for each data point and the associated standard deviation).

FIG. 54 is a line graph depicting the % of CE released over time for Example 34J (see Table 49 for each data point and the associated standard deviation).

### DETAILED DESCRIPTION OF THE INVENTION

The present invention relates to a tablet-in-tablet composition having improved characteristics, including content uniformity (C.U.), compared to compositions containing similar compounds such as compositions having one or more active layers coated via suspension layering or sugar coating. The invention therefore includes methods for producing and testing such tablets, e.g., a tablet that includes a core containing an estrogen and an outer layer containing a selective estrogen receptor modulator (SERM) or a progestational agent.

One formulation of the tablet-in-tablet composition includes a hydrophilic gel-forming polymer in the outer tablet layer, which slows the release of active pharmaceutical ingredient (API) from the outer tablet layer. This formulation further includes diluent and binder components, and may also include an antioxidant component and/or a lubricant component. A second formulation contains one or more of a diluent component, a binder component, and a hydrophilic gel-forming polymer component, allowing for more rapid release of API from the outer tablet layer than in the first formulation. This second formulation may also include an antioxidant component and/or a lubricant component. A third formulation includes diluent, binder, and disintegrant components in the outer tablet layer. The disintegrant component provides for almost immediate release of API from the outer tablet layer. This third formulation may also include an antioxidant component and/or a lubricant component. Processes for making these formulations of the tablet-in-tablet composition are disclosed herein.

Because of the excellent content uniformity of each layer of the tablet-in-tablet composition, the delivery of each API is improved, e.g., compared to a composition in which the estrogen and SERM or progestin are compounded together, or where an active layer is applied via suspension coating or sugar coating. Typically, a tablet-in-tablet composition as described herein will have C.U. of less than or equal to 3.5%. The weight variation of a tablet-in-tablet composition as described herein will typically be less than or equal to 2%.

The methods and compositions provided herein permit varied formulation of excipients in the tablet-in-tablet composition, which is advantageous for readily testing different in vitro release characteristics, which can result in different in vivo outcomes depending on the ratio and amount of excipients formulated in the chosen composition. In part, because the compounds are formulated in separate layers of the tablet-in-tablet composition, controlled release rates can be tailored for each compound in the tablet-in-tablet composition. Known compositions display more variable C.U., which results in more variability of each component of the composition and accordingly increases the variability of the release rate of each compound. Thus the disclosed tablet-in-tablet composition is an improvement over currently available compositions of an estrogen and SERM or estrogen and progestin. In addition, the disclosed tablet-in-tablet composition can be readily manufactured, e.g., with varying dosages of each compound, therefore adapting various formulations for specific intended uses or release characteristics, e.g., for treating infertility, perimenopause, menopause, and postmenopausal symptoms. The disclosed tablet-in-tablet composition may be formulated for different dissolution rates of API from the table core and the outer tablet layer, allowing for further adaptation of various formulations for specific intended uses. The estrogen/SERM and estrogen/progestin tablets described herein thus have better tablet to tablet control than compositions that are currently available and therefore can provide better treatment for patients using such compositions.

Because of the finding that the compositions described herein can be formulated to make an effective composition with C.U. that is generally improved over currently available compositions, additional advantages include the ease of production of a tablet-in-tablet composition comprising an estrogen and a SERM or an estrogen and a progestin. It is commercially practical to make such tablets, including more economical, e.g., because the manufacturing time for tableting is less than for suspension layering or sugar coating. Furthermore, the disclosed tablet-in-table composition employs tableting equipment that is less likely to malfunction than spray coating equipment. The disclosed compositions can be as stable as, or more stable than, previously known formulations using suspension layering or sugar coating. Finally, the compositions disclosed herein can be formulated to have diminished or none of the odor that is characteristic odor of conjugated estrogen preparations, e.g., obtained from pregnant mare urine. Accordingly, the compositions provided herein can be more palatable than known coated compositions.

### DEFINITIONS

As used herein, the term “about” means plus or minus 10% of the value, unless otherwise indicated herein.

As used herein, the term “alginic acid” refers to a naturally occurring hydrophilic colloidal polysaccharide obtained from the various species of seaweed, or synthetically modified polysaccharides thereof.

As used herein, the term “sodium alginate” refers to a sodium salt of alginic acid and can be formed by reaction of alginic acid with a sodium containing base such as sodium hydroxide or sodium carbonate. As used herein, the term “potassium alginate” refers to a potassium salt of alginic acid and can be formed by reaction of alginic acid with a potassium containing base such as potassium hydroxide or potassium carbonate. As used herein, the term “calcium alginate” refers to a calcium salt of alginic acid and can be formed by reaction of alginic acid with a calcium containing base such as calcium hydroxide or calcium carbonate. Suitable sodium alginates, calcium alginates, and potassium alginates include, but are not limited to, those described in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety. Suitable sodium alginates, include, but are not limited to, KELCOSOL® (ISP, Wayne, N.J.), KELFONE™ LVCR and HVCR (ISP, Wayne, N.J.), MANUCOL® (ISP, Wayne, N.J.), and PROTANOL™ (FMC Biopolymer, Philadelphia, Pa.).

As used herein, the phrase “apparent viscosity” refers to a viscosity measured by the USP method.

As used herein, the abbreviation “BZA” refers to bazedoxifene acetate.

As used herein, the term “calcium phosphate” refers to monobasic calcium phosophate, dibasic calcium phosphate or tribasic calcium phosphate.

As used herein, the abbreviation “CE” refers to conjugated estrogens.

Cellulose, cellulose floc, powdered cellulose, microcrystalline cellulose, silicified microcrystalline cellulose, hydroxyethylcellulose, hydroxypropylcellulose, hydroxypropylmethylcellulose, and methylcellulose include, but are not limited to, those described in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety. As used herein, cellulose refers to natural cellulose. The term “cellulose” also refers to celluloses that have been modified with regard to molecular weight and/or branching, particularly to lower molecular weight. The term “cellulose” further refers to celluloses that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable powdered celluloses for use in the invention include, but are not limited to ARBOCEL® (JRS Pharma, Patterson, N.Y.), SANACEL® (CFF GmbH), and SOLKA-FLOC® (International Fiber Corp.).

Suitable microcrystalline celluloses include, but are not limited to, the AVICEL® PH series (FMC Biopolymer, Philadelphia, Pa.), CELEX™ (ISP, Wayne, N.J.), CELPHERE® (Asahi Kasei, Tokyo, Japan), CEOLUS® KG (Asahi Kasei, Tokyo, Japan), and VIVAPUR® (JRS Pharma, Patterson, N.Y.). In some embodiments, the microcrystalline cellulose is AVICEL® PH 200.

As used herein, the term “hydroxyethylcellulose” refers to a cellulose ether with pendant hydroxyethyl groups of formula HO—CH2-CH2-, attached to the cellulose via an ether linkage. Suitable hydroxyethylcelluloses include, but are not limited to, CELLOSIZE® HEC (Dow Chemical Co., Midland, Mich.), NATROSOL® (Hercules, Inc., Wilmington, Del.), and TYLOSE® PHA (Clariant Corp., Muttenz, Switzerland).

As used herein, the term “hydroxypropylcellulose” refers to a cellulose that has pendant hydroxypropoxy groups, and includes both high- and low-substituted hydroxypropylcellulose. In some embodiments, the hydroxypropylcellulose has about 5% to about 25% hydroxypropyl groups. Suitable hydroxypropylcelluloses include, but are not limited to, the KLUCEL® series (Hercules, Inc., Wilmington, Del.), the METHOCEL® series (Dow Chemical Co., Midland, Mich.), the NISSO HPC series (Nisso America Inc., New York, N.Y.), the METOLOSE® series (Shin-Etsu, Tokyo, Japan), and the LH series, including LHR-11, LH-21, LH-31, LH-20, LH-30, LH-22, and LH-32 (Shin-Etsu, Tokyo, Japan).

As used herein, the term “methyl cellulose” refers to a cellulose that has pendant methoxy groups. Suitable methyl celluloses include, but are not limited to CULMINAL® MC (Hercules, Inc., Wilmington, Del.).

As used herein, the term “carboxymethylcellulose sodium” refers to a cellulose ether with pendant groups of the formula Na⁺⁻O—C(O)—CH₂—, attached to the cellulose via an ether linkage. Suitable carboxymethylcellulose sodium polymers include, but are not limited to, AKUCELL® (Akzo Nobel, Amsterdam, The Netherlands), AQUASORB® (Hercules, Inc., Wilmington, Del.), BLANOSE® (Hercules, Inc., Wilmington, Del.), FINNFIX® (Noviant, Arnhem, The Netherlands), NYMEL™ (Noviant, Arnhem, The Netherlands), and TYLOSE® CB (Clariant Corp., Muttenz, Switzerland).

As used herein, the term “compressed outer tablet layer” means that the outer tablet layer of the tablet-in-tablet composition is formed by compression of a solid mixture, such as a direct blend, dry granulation, or wet granulation, rather than forming the outer layer by coating with a suspension or solution. Suitable compression techniques include, but are not limited to, compression with the 11 mm round convex tooling utilizing a Kilian RUD compression machine. In some embodiments, the compressed outer tablet layer without core tablet portion is compressed to a hardness of 2 kp to 7 kp. To perform the measurement, only the outer tablet layer blend was compressed and its hardness measured.

As used herein, “content uniformity” is measured by use of USP Method <905> (General Chapters, Uniformity of Dosage Forms), unless otherwise indicated. In this context, a plurality refers to ten or more tablet-in-tablet compositions.

As used herein, the term “copovidone” refers to a copolymer of vinylpyrrolidone and vinyl acetate, wherein the vinyl acetate monomers may be partially hydrolyzed. Suitable copovidone polymers include, but are not limited to KOLLIDON® VA 64 (BASF, Florham Park, N.J.), LUVISKOL® VA (BASF, Florham Park, N.J.), PLASDONE® S-630 (ISP, Wayne, N.J.), and MAJSAO® CT (Cognis, Monheim, Germany).

As used herein, the term “core” in the phrases “core filler/diluent component”, “core filler/binder component”, “core hydrophilic gel-forming polymer component”, and “core lubricant component” is used to specify that the component is present in the core tablet portion of the tablet-in-tablet composition.

As used herein, the term “croscarmellose calcium” refers to a crosslinked polymer of carboxymethylcellulose calcium.

As used herein, the term “croscarmellose sodium” refers to a crosslinked polymer of carboxymethylcellulose sodium. In some embodiments, the croscarmellose sodium is Ac.Di.Sol (FMC Biopolymer, Philadelphia, Pa.).

As used herein, the term “crospovidone” refers to a crosslinked polymer of polyvinylpyrrolidone. Suitable crospovidone polymers include, but are not limited to POLYPLASDONE® XL-10 (ISP, Wayne, N.J.) and KOLLIDON® CL and CL-M (BASF, Florham Park, N.J.).

As used herein, the phrase “dissolution profile” refers to the amount of active pharmacological agent dissolved under specified conditions in a specified period of time.

As used herein, the term “fatty acid”, employed alone or in combination with other terms, refers to an aliphatic acid that is saturated or unsaturated. In some embodiments, the fatty acid is a mixture of different fatty acids. In some embodiments, the fatty acid has between about eight to about thirty carbons on average. In some embodiments, the fatty acid has about eight to about twenty-four carbons on average. In some embodiments, the fatty acid has about twelve to about eighteen carbons on average. Suitable fatty acids include, but are not limited to, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, benhenic acid, isobehenic acid, and arachidonic acid, or mixtures thereof.

As used herein, the term “fatty acid ester” refers to a compound formed between a fatty acid and a hydroxyl containing compound. In some embodiments, the fatty acid ester is a sugar ester of fatty acid. In some embodiments, the fatty acid ester is a glyceride of fatty acid. In some embodiments, the fatty acid ester is an ethoxylated fatty acid ester.

As used herein, the term “fatty alcohol”, employed alone or in combination with other terms, refers to an aliphatic alcohol that is saturated or unsaturated. In some embodiments, the fatty alcohol is a mixture of different fatty alcohols. In some embodiments, the fatty alcohol has between about eight to about thirty carbons on average. In some embodiments, the fatty alcohol has about eight to about twenty-four carbons on average. In some embodiments, the fatty alcohol has about twelve to about eighteen carbons on average. Suitable fatty alcohols include, but are not limited to, stearyl alcohol, lauryl alcohol, palmityl alcohol, palmitolyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “filler/binder component” refers to one or more substances that can act as fillers and/or binders, although the substances may have additional, unspecified benefits.

As used herein, the term “filler/diluent component” refers to one or more substances that act to dilute the active pharmacological agent to the desired dosage and/or that act as a carrier for the active pharmacological agent, although the substances may have additional, unspecified benefits.

As used herein, the term “first solid mixture” in the phrases “first solid mixture filler/diluent component”, “first solid mixture filler/binder component”, “first solid mixture hydrophilic gel-forming polymer component”, and “first solid mixture lubricant component” is used to specify that the component is present in the first solid mixture used to form the core tablet portion of the tablet-in-tablet composition.

As used herein, the term “gelatin” refers to any material derived from boiling the bones, tendons, and/or skins of animals, or the material known as agar, derived from seaweed. The term “gelatin” also refers to any synthetic modifications of natural gelatin. Suitable gelatins include, but are not limited to, Byco (Croda Chemicals, East Yorkshire, UK) and CRYOGEL™ and INSTAGEL™ (Tessenderlo, Brussels, Belgium), and the materials described in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety.

As used herein, the term “gum arabic” refers to natural, or synthetically modified, arabic gum. As used herein, the term “gum tragacanath” refers to natural, or synthetically modified, tragacanath gum. As used herein, the term “gum acacia” refers to natural, or synthetically modified, acacia gum. Suitable gum arabic, gum tragacanath, and gum acacia include, but are not limited to, those described in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety.

As used herein, hardness is measured on a standard tablet hardness tester, such as a Schleuniger 2E tablet hardness tester on a test area width of 35 mm or 15 mm.

As used herein, the term “hydrophilic gel-forming polymer component” refers to one or more hydrophilic polymers, wherein the dry polymer is capable of swelling in the presence of aqueous media to form a highly viscous gelatinous mass.

As used herein, the term “lubricant component” refers to one or more substances that aids in preventing the pharmaceutical formulations from sticking to equipment during processing and/or that improves powder flow of the formulation during processing.

Suitable mannitols include, but are not limited to, PHARMMANNIDEX™ (Cargill, Minneapolis, Minn.), PEARLITOL® (Roquette Freres, Lestrem, France), and MANNOGEM™ (SPI Polyols, New Castle, Del.).

As used herein, the phrase “mean dissolution profile” means that the percentage of each active pharmacological agent which dissolves after specified period of time under specified conditions is first measured for each composition in a plurality. The mean percentage of active pharmacological agent released at a given time for the plurality is then calculated by adding the percentages of active pharmacological agent released at a given time for each composition and then dividing by the number of compositions in the plurality.

As used herein, the phrase “mean of % of the estrogen released per composition” means that the percentage of estrogen which dissolves after specified period of time under specified conditions is first measured for each composition in a plurality. The mean percentage of estrogen released at a given time for the plurality is then calculated by adding the percentages of estrogen released at a given time for each composition and then dividing by the number of compositions in the plurality.

As used herein, the phrase “mean of % of the therapeutic agent released per composition” means that the percentage of one of the therapeutic agents which dissolves after specified period of time under specified conditions is first measured for each composition in a plurality. The mean percentage of therapeutic agent released at a given time for the plurality is then calculated by adding the percentages of the therapeutic agent released at a given time for each composition and then dividing by the number of compositions in the plurality.

As used herein, the term “metallic alkyl sulfate” refers to a metallic salt formed between inorganic base and an alkyl sulfate compound. In some embodiments, the metallic alkyl sulfate has about eight carbons to about eighteen carbons. In some embodiments, metallic alkyl sulfate is a metallic lauryl sulfate. In some embodiments, the metallic alkyl sulfate is sodium lauryl sulfate.

As used herein, the term “metal carbonate” refers to any metallic carbonate, including, but not limited to sodium carbonate, calcium carbonate, and magnesium carbonate, and zinc carbonate.

As used herein, the term “metallic stearate” refers to a metal salt of stearic acid. In some embodiments, the metallic stearate is calcium stearate, zinc stearate, or magnesium stearate. In some embodiments, the metallic stearate is magnesium stearate.

As used herein, the term “mineral oil” refers to both unrefined and refined (light) mineral oil. Suitable mineral oils include, but are not limited to, the AVATECH™ grades (Avatar Corp., University Park, Ill.), DRAKEOL™grades (Penreco, Dickinson, Tex.), SIRIUS™ grades (Royal Dutch Shell, The Hague, Netherlands), and the CITATION™ grades (available from Avatar Corp., University Park, Ill.).

As used herein, the abbreviation “MPA” refers to medroxyprogestrone acetate.

As used herein, the term “outer layer” in the phrases “outer layer filler/diluent component”, “outer layer filler/binder component”, “outer layer hydrophilic gel-forming polymer component”, “outer layer lubricant component”, “outer layer wetting agent component”, and “outer layer disintegrant component” is used to specify that the component is present in the compressed outer tablet layer portion of the tablet-in-tablet composition.

As used herein, the term “plurality” refers to two or more tablet-in-tablet compositions, unless otherwise indicated. In some embodiments, a plurality refers to six or more tablet-in-tablet compositions. In the context of embodiments directed to content uniformity, a plurality refers to ten or more tablet-in-tablet compositions. In the context of embodiments directed to weight variation, a plurality refers to 100 or more tablet-in-tablet compositions. In some embodiments, the plurality is derived from a single manufacturing batch of compositions.

As used herein, the term “polyethoxylated fatty acid ester” refers to a monoester or diester, or mixture thereof, derived from the ethoxylation of a fatty acid. The polyethoyxylated fatty acid ester can contain free fatty acids and polyethylene glycol as well. Fatty acids useful for forming the polyethoxylated fatty acid esters include, but are not limited to, those described herein. Suitable polyethoxylated fatty acid esters include, but are not limited to, EMULPHOR™ VT-679 (stearic acid 8.3 mole ethoxylate, available from Stepan Products, Northfield, Ill.), the ALKASURF™ CO series (Alkaril Chemicals, Mississauga, Canada), macrogol 15 hydroxystearate, SOLUTOL™ HS15 (BASF, Florham Park, N.J.), and the polyoxyethylene stearates listed in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety.

As used herein, the term “polyethylene glycol” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (Dow Chemical Co., Midland, Mich.), the Lipoxol™ series (Brenntag, Ruhr, Germany), the Lutrol™ series (BASF, Florham Park, N.J.), and the Pluriol™ series (BASF, Florham Park, N.J.).

As used herein, the term “polyethylene glycol-polypropylene glycol copolymer” refers to a copolymer that has both oxyethylene monomer units and oxypropylene monomer units. Suitable polyethylene glycol-polypropylene glycol copolymers for use in the invention can be of any chain length or molecular weight, and can include branching. The chain ends may have a free hydroxyl group or may have one or more hydroxyl groups etherified with a lower alkyl or carboxy group. The polyoxyethylene-polyoxypropylene copolymers can also include other monomers which were copolymerized and which form part of the backbone. For example, butylene oxide can be copolymerized with ethylene oxide and propylene oxide to form polyethylene glycol-polypropylene glycol copolymers useful in the present invention. In some embodiments, the polyethylene glycol-polypropylene glycol copolymer is a block copolymer, wherein one block is polyoxyethylene and the other block is polyoxypropylene. Suitable polyethylene glycol-polypropylene glycol copolymer copolymers include, but are not limited to, Poloxamer 108, 124, 188, 217, 237, 238, 288, 338, 407, 101, 105, 122, 123, 124, 181, 182, 183, 184, 212, 231, 282, 331, 401, 402, 185, 215, 234, 235, 284, 333, 334, 335, and 403. Other suitable polyoxyethylene-polyoxypropylene copolymers include, but are not limited to, DOWFAX® Nonionic surfactants (Dow Chemical Co., Midland, Mich.), the DOWFAX® N-Series surfactants (Dow Chemical Co., Midland, Mich.), LUTROL™ surfactants such as LUTROL MICRO 68 (BASF, Florham Park, N.J.), and SYNPERONIC™ surfactants (Uniqema, Bromborough, UK).

As used herein, the term “polyethylene oxide castor oil derivatives” refers to a compound formed from the ethoxylation of castor oil, wherein at least one chain of polyethylene glycol is covalently bound to the castor oil. The castor oil may be hydrogenated or unhydrogenated. Synonyms for polyethylene oxide castor oil derivatives include, but are not limited to, polyoxyl castor oil, hydrogenated polyoxyl castor oil, macrogolglyceroli ricinoleas, macrogolglyceroli hydroxystearas, polyoxyl 35 castor oil, and polyoxyl 40 hydrogenated castor oil. Suitable polyethylene oxide castor oil derivatives include, but are not limited to, the NIKKOL™ HCO series (Nikko Chemicals Co. Ltd., Tokyo, Japan), such as NIKKOL™ HCO-30, HC-40, HC-50, and HC-60 (polyethylene glycol-30 hydrogenated castor oil, polyethylene glycol-40 hydrogenated castor oil, polyethylene glycol-50 hydrogenated castor oil, and polyethylene glycol-60 hydrogenated castor oil, EMULPHOR™ EL-719 (castor oil 40 mole-ethoxylate, Stepan Products, Northfield, Ill.), the CREMOPHORE™ series (BASF, Florham Park, N.J.), which includes CREMOPHORE RH40, RH60, and EL35 (polyethylene glycol-40 hydrogenated castor oil, polyethylene glycol-60 hydrogenated castor oil, and polyethylene glycol-35 hydrogenated castor oil, respectively), and the EMULGIN® RO and HRE series (Cognis PharmaLine, Monheim, Germany). Other suitable polyethylene oxide castor oil derivatives include those listed in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety.

As used herein, the term, “polyethylene oxide sorbitan fatty acid ester” refers to a compound, or mixture thereof, derived from the ethoxylation of a sorbitan ester. As used herein, the term “sorbitan ester” refers to a compound, or mixture of compounds, derived from the esterification of sorbitol and at least one fatty acid. Fatty acids useful for deriving the polyethylene oxide sorbitan esters include, but are not limited to, those described herein. In some embodiments, the polyethylene oxide portion of the compound or mixture has about 2 to about 200 oxyethylene units. In some embodiments, the polyethylene oxide portion of the compound or mixture has about 2 to about 100 oxyethylene units. In some embodiments, the polyethylene oxide portion of the compound or mixture has about 4 to about 80 oxyethylene units. In some embodiments, the polyoxyethylene portion of the compound or mixture has about 4 to about 40 oxyethylene units. In some embodiments, the polyethylene oxide portion of the compound or mixture has about 4 to about 20 oxyethylene units. Suitable polyethylene oxide sorbitan esters include, but are not limited to the TWEEN series (Uniqema, Bromborough, UK), which includes Tween 20 (POE(20) sorbitan monolaurate), 21 (POE(4) sorbitan monolaurate), 40 (POE(20) sorbitan monopalmitate), 60 (POE(20) sorbitan monostearate), 60K (POE(20) sorbitan monostearate), 61 (POE(4) sorbitan monostearate), 65 (POE(20) sorbitan tristearate), 80 (POE(20) sorbitan monooleate), 80K (POE(20) sorbitan monooleate), 81 (POE(5) sorbitan monooleate), and 85 (POE(20) sorbitan trioleate). As used herein, the abbreviation “POE” refers to polyethylene oxide. The number following the POE abbreviation refers to the number of oxyethylene repeat units in the compound. Other suitable polyethylene oxide sorbitan esters include the polyethylene oxide sorbitan fatty acid esters listed in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety.

As used herein, the term “polyglycolized glycerides” refers to the products formed from the esterification of polyethylene glycol, glycerol, and fatty acids; the transesterification of glycerides and polyethylene glycol; or the ethoxylation of a glyceride of a fatty acid. As used herein, the term “polyglycolized glycerides” can, alternatively or additionally, refer to mixtures of monoglycerides, diglycerides, and/or triglycerides with monoesters and/or diesters of polyethylene glycol. Polyglycolized glycerides can be derived from the fatty acids, glycerides of fatty acids, and polyethylene glycols described herein. The fatty ester side-chains on the glycerides, monoesters, or diesters can be of any chain length and can be saturated or unsaturated. The polyglycolized glycerides can contain other materials as contaminants or side-products, such as, but not limited to, polyethylene glycol, glycerol, and fatty acids.

As used herein, the term “polyvinyl alcohol” refers to a polymer formed by partial or complete hydrolysis of polyvinyl acetate. Suitable polyvinyl alcohols include, but are not limited to, the AIRVOL® series (Air Products, Allentown, Pa.), the ALCOTEX® series (Synthomer LLC, Powell, Ohio), the ELVANOL® series (DuPont, Wilmington, Del.), the GELVATOL® series (Burkard), and the GOHSENOL® series (Nippon Gohsei, Osaka, Japan).

As used herein, the term “polyvinylpyrrolidone” refers to a polymer of vinylpyrrolidone. In some embodiments, the polyvinylpyrrolidone contains one or more additional polymerized monomers. In some embodiments, the additional polymerized monomer is a carboxy containing monomer. In some embodiments, the polyvinylpyrrolidone is povidone. In some embodiments, the polyvinylpyrrolidone has a molecular weight between 2500 and 3 million. In some embodiments, the polyvinylpyrrolidone is povidone K12, K17, K25, K30, K60, K90, or K120. Suitable polyvinylpyrrolidone polymers include, but are not limited to, the KOLLIDONE™ series (BASF, Florham Park, N.J.) and the PLASDONE™ series (ISP, Wayne, N.J.).

As used herein, the term “propylene glycol fatty acid ester” refers to an monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: LAUROGLYCOL™ FCC and 90 (Gattefosse Corp., Paramus, N.J.); propylene glycol caprylates: CAPRYOL™ PGMC and 90 (Gattefosse Corp., Paramus, N.J.); and propylene glycol dicaprylocaprates: LABRAFAC™ PG (Gattefosse Corp., Paramus, N.J.).

As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient. Pharmaceutically acceptable salts, including mono- and bi-salts, include, but are not limited to, those derived from organic and inorganic acids such as, but not limited to, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties.

As used herein, the term “quaternary ammonium compound” refers a compound that contains at least one quaternary ammonium group. Particularly useful quaternary ammonium compounds are those that are capable of emulsifying, solubilizing, or suspending hydrophobic materials in water. Other quaternary ammonium compounds useful in the invention are those that can enhance bioavailability of the active pharmacological agent when administered to the patient. Suitable quaternary ammonium compounds include, but are not limited to, 1,2-dioleyl-3-trimethylammonium propane, dimethyldioctadecylammonium bromide, N-[1-(1,2-dioleyloxy)propyl]-N,N,N-trimethylammonium chloride, 1,2-dioleyl-3-ethylphosphocholine, or 3-β-[N—[(N′,N′-dimethylamino)ethan]carbamoyl]cholesterol. Other suitable quaternary ammonium compounds include, but are not limited to, Stepanquat™ 50NF and 65NF (n-alkyl dimethyl benzyl ammonium chloride, Stepan Products, Northfield, Ill.).

As used herein, “released” means dissolved under the specified conditions.

As used herein, the term “second solid mixture” in the phrases “second solid mixture filler/diluent component”, “second solid mixture filler/binder component”, “second solid mixture hydrophilic gel-forming polymer component”, “second solid mixture lubricant component”, “second solid mixture wetting agent component”, “second solid mixture antioxidant component” and “second solid mixture disintegrant component” is used to specify that the component is present in the second solid mixture used to form the compressed outer tablet layer portion of the tablet-in-tablet composition.

As used herein, the term “silicified microcrystalline cellulose” refers to a synergistic intimate physical mixture of silicon dioxide and microcrystalline cellulose. Suitable silicified microcrystalline celluloses include, but are not limited to, the PROSOLV® line of products, including PROSOLV® 90 (JRS Pharma, Patterson, N.Y.).

Suitable sorbitols include, but are not limited to, PHARMSORBIDEX™E420 (Cargill, Minneapolis, Minn.), LIPONIC® 70-NC and 76-NC (Lipo Chemical, Paterson, N.J.), NEOSORB® (Roquette Freres, Lestrem, France), PARTECH™ SI (Merck, Whitehouse Station, N.J.), and SORBOGEM® (SPI Polyols, New Castle, Del.).

Starches and sodium starch glycolate include, but are not limited to, those described in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety.

As used herein, the term “starch” refers to any type of natural or modified starch including, but not limited to, maize starch (also known as corn starch or maydis amylum), potato starch (also known as solani amylum), rice starch (also known as oryzae amylum), wheat starch (also known as tritici amylum), and tapioca starch. The term “starch” also refers to starches that have been modified with regard to molecular weight and branching. The term “starch” further refers to starches that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable sodium starch glycolates include, but are not limited to, EXPLOTAB® (JRS Pharma, Patterson, N.Y.), GLYCOLYS® (Roquette Freres, Lestrem, France), PRIMOJEL® (DMV International), and VIVASTAR® (JRS Pharma, Patterson, N.Y.).

Suitable pregelatinized starches include, but are not limited to, LYCATAB® C and PGS (Roquette Freres, Lestrem, France), MERIGEL™ (Brenntag, Ruhr, Germany), NATIONAL™ 78-1551 (National Starch & Chemical Co., Bridgewater, N.J.), SPRESS® B820 (Grain Processing Corp., Muscatine, Iowa), and Starch 1500 (Colorcon, West Point, Pa.).

As used herein, the phrase “substantially equal to” means the value plus or minus 20% of the value.

As used herein, the term “substantially as shown” means that the profile is plus or minus 2σ (twice the standard deviation) of the value for each point of the figure (the standard deviation, σ, for the individual points in the figures are shown in Tables 20-23, 30-31, and 49-52).

As used herein, the term “sugar ester of fatty acid” refers to an ester compound formed between a fatty acid and carbohydrate or sugar molecule. In some embodiments, the carbohydrate is glucose, lactose, sucrose, dextrose, mannitol, xylitol, sorbitol, maltodextrin and the like. Suitable sugar esters of fatty acids include, but are not limited to sucrose fatty acid esters (such as those available from Mitsubishi Chemical Corp., Tokyo, Japan).

As used herein, the term “tablet-in-tablet composition” refers to a pharmaceutical dosage form comprising an outer layer, which has been compressed onto a core tablet, such that the core tablet is completely surrounded by the compressed outer tablet layer and such that no surface of the core tablet remains visible.

As used herein, the phrase “under estrogen dissolution conditions” refers to subjecting a composition of the invention to USP Apparatus 2, at 50 rpm in 900 mL of 0.02 M sodium acetate buffer of pH 4.5, in order to measure the amount of estrogen which dissolves at each various time(s). In some embodiments, the core tablet comprises at least one conjugated estrogen.

As used herein, the phrase “under type I therapeutic agent conditions” refers to subjecting a composition of the invention to USP Apparatus 2, at 50 rpm in 900 mL of 0.54% sodium lauryl sulfate in water, in order to measure the amount of therapeutic agent which dissolves at each time. In some embodiments, the therapeutic agent is medroxyprogesterone acetate.

As used herein, the phrase “under type II therapeutic agent conditions” refers to subjecting a composition of the invention to USP Apparatus 1 (basket), at 75 rpm in 900 mL of 10 mM acetic acid solution with 0.2% polysorbate 80 (Tween 80) at 37° C. for a period of 60 minutes, changing the speed to 250 rpm at 80 minutes, in order to measure the amount of therapeutic agent which dissolves at each time. In some embodiments, the compressed outer tablet layer comprises bazedoxifene acetate.

As used herein, the term “vegetable oil” refers to naturally occurring or synthetic oils, which may be refined, fractionated or hydrogenated, including triglycerides. Suitable vegetable oils include, but are not limited to, castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, MIGLYOL™ 810 and 812 (Dynamit Nobel Chemicals, Sweden) NEOBEE™ M5 (Drew Chemical Corp., Boonton, N.J.), ALOFINE™ (Jarchem Industries, Newark, N.J.), the LUBRITAB™ series (JRS Pharma, Patterson, N.Y.), the STEROTEX™ (Abitec Corp., Columbus, Ohio), SOFTISAN™ 154 (Sasol, Johannesburg, South Africa), CRODURET™ (Croda Chemicals, East Yorkshire, UK), FANCOL™ (the Fanning Corp., Chicago, Ill.), CUTINA™ HR (Cognis, Monheim, Germany), SIMULSOL™ (CJ Petrow Chemicals, Johannesburg, South Africa), EMCON™ CO (Amisol Co., Toronto, Canada), LIPVOL™ CO, SES, and HS-K (Lipo Chemical, Paterson, N.J.), and STEROTEX™ HM (Abitec Corp., Columbus, Ohio). Other suitable vegetable oils, including sesame, castor, corn, and cottonseed oils, include those listed in R. C. Rowe and P. J. Shesky, Handbook of Pharmaceutical Excipients, (Great Britain: Pharmaceutical Press; Washington, D.C.: American Pharmacists Association, 5th ed.) (2006), which is incorporated herein by reference in its entirety.

As used herein, “weight variation” is measured by use of USP Method <905> (General Chapters, Uniformity of Dosage Forms), unless otherwise indicated. In this context, a plurality refers to 100 or more tablet-in-tablet compositions.

As will be appreciated, some components of the pharmaceutical formulations of the invention can possess multiple functions. For example, a given component can act as both a filler/diluent and a disintegrant. In some such cases, the function of a given component can be considered singular, even though its properties may allow multiple functionalities.

In a first aspect, the present invention provides a tablet-in-tablet composition comprising:

a) a core tablet comprising:
one or more estrogens;
a core filler/diluent component comprising from about 30% to about 85% by weight of the core tablet;
a core filler/binder component comprising from about 1% to about 30% by weight of the core tablet;
a core hydrophilic gel-forming polymer component comprising from about 1% to about 40% by weight of the core tablet; and
optionally, a core lubricant component comprising from about 0.01% to about 2% by weight of the core tablet; and

b) a compressed outer tablet layer comprising:
one or more therapeutic agents selected from the group consisting of selective estrogen receptor modulators and progestational agents;
an outer layer filler/diluent component comprising from about 10% to about 80% by weight of the compressed outer tablet layer;
an outer layer filler/binder component comprising from about 1% to about 70% by weight of the compressed outer tablet layer;
an outer layer hydrophilic gel-forming polymer component comprising from about 1% to about 70% by weight of the compressed outer tablet layer;
optionally, an antioxidant component comprising from about 0.01% to about 4% by weight of the compressed outer tablet layer; and
optionally, an outer layer lubricant component comprising from about 0.01% to about 2% by weight of the compressed outer tablet layer.

In a second aspect, the present invention provides a tablet-in-tablet composition comprising:

a) a core tablet comprising:
one or more estrogens;
a core filler/diluent component comprising from about 30% to about 85% by weight by weight of the core tablet;
a core filler/binder component comprising from about 1% to about 30% by weight of the core tablet;
a core hydrophilic gel-forming polymer component comprising from about 1% to about 40% by weight of the core tablet; and
optionally, a core lubricant component comprising from about 0.01% to about 2% by weight of the core tablet; and

b) a compressed outer tablet layer comprising:
one or more therapeutic agents selected from the group consisting of selective estrogen receptor modulators and progestational agents;
a pharmaceutically acceptable carrier component comprising from about 60% to about 99.9% by weight of the compressed outer tablet layer, wherein the pharmaceutically acceptable carrier component optionally comprises one or more of an outer layer filler/diluent component, an outer layer filler/binder component, and an outer layer hydrophilic gel-forming polymer component;
optionally, an outer layer lubricant component comprising from about 0.01% to about 2% by weight of the compressed outer tablet layer; and
optionally, an antioxidant component comprising from about 0.01% to about 4% by weight of the compressed outer tablet layer.

In a third aspect, the present invention provides a tablet-in-tablet composition comprising:

a) a core tablet comprising:
one or more estrogens;
a core filler/diluent component comprising from about 30% to about 85% by weight by weight of the core tablet;
a core filler/binder component comprising from about 1% to about 30% by weight of the core tablet;
a core hydrophilic gel-forming polymer component comprising from about 1% to about 40% by weight of the core tablet; and
optionally, a core lubricant component comprising from about 0.01% to about 2% by weight of the core tablet; and

b) a compressed outer tablet layer comprising:
one or more therapeutic agents selected from the group consisting of selective estrogen receptor modulators and progestational agents;
an outer layer filler/diluent component comprising from about 25% to about 65% by weight of the compressed outer tablet layer;
an outer layer filler/binder component comprising from about 20% to about 50% by weight of the compressed outer tablet layer;
a disintegrant component comprising from about 2% to about 15% by weight of the compressed outer tablet layer;
optionally, an outer layer wetting agent component comprising from about 0.01% to about 4% of the compressed outer tablet layer;
optionally, an outer layer lubricant component comprising from about 0.01% to about 2% by weight of the compressed outer tablet layer; and
optionally, an antioxidant component comprising from about 0.01% to about 4% by weight of the compressed outer tablet layer.

Unless otherwise specified, each of the embodiments described herein can be provided for the first, second, and third aspects of the invention.

As used herein, an estrogen is a natural or synthetic substance which dISP, Wayne, NJIays estrogenic activity. In some embodiments, the core tablet comprises one or more estrogens which are selected from the group consisting of estradiol, estradiol benzoate, estradiol valerate, estradiol cypionate, estradiol heptanoate, estradiol decanoate, estradiol acetate, estradiol diacetate, 17α-estradiol, ethinylestradiol, ethinylestradiol 3-acetate, ethinylestradiol 3-benzoate, estriol, estriol succinate, polyestrol phosphate, estrone, estrone acetate, estrone sulfate, piperazine estrone sulfate, quinestrol, mestranol, and conjugated equine estrogens, or other pharmaceutically acceptable ester and ether thereof. In some embodiments, the core tablet comprises at least one conjugated estrogen. In some embodiments, the core tablet comprises combinations of estrogens.

As used herein, the terms “conjugated estrogen” and “conjugated estrogens” (“CE”) includes both natural and synthetic conjugated estrogens, such as the compounds described in the United States Pharmacopeia (USP 23), as well as other estrogens so considered by those skilled in the art. Further, “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include: 17-α and β-dihydroequilin, equilenin, 17-α and β-dihydroequilenin, estrone, 17-β-estradiol, and their sodium sulfate esters.

Although CE are typically a mixture of estrogens, such as estrone and equilin, the core tablet material may be formulated to either utilize such a mixture, or to include only selected or individual estrogenic components. These CE may be of synthetic or natural origin. Examples of synthetically produced estrogens include, inter alia, sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate, estropipate and ethinyl estradiol. The alkali metal salts of 8,9-dehydroestrone and the alkali metal salts of 8,9-dehydroestrone sulfate ester, as described in U.S. Pat. No. 5,210,081, which is herein incorporated by reference, also may be used. Naturally occurring CE are usually obtained from pregnant mare urine and then are processed and may be stabilized. Examples of such processes are set forth in U.S. Pat. Nos. 2,565,115 and 2,720,483, each of which are hereby incorporated by reference.

Many CE products are commercially available. One such CE product is the naturally occurring CE product known as PREMARIN® (Wyeth, Madison, N.J.). Another commercially available CE product prepared from synthetic estrogens is CENESTIN® (Duramed Pharmaceuticals, Inc., Cincinnati, Ohio). The specific CE dose included in the core tablet material may be any dosage required to achieve a specific therapeutic effect, and may vary depending on the specific treatment indicated, and on the specific CE included in the tablet. In some embodiments, the CE is a CE dessication with a sugar material such as lactose, sucrose, and the like. In some embodiments, the CE is a CE dessication with lactose.

As used herein, the term “progestational agent” refers to a natural or synthetic substance which has progestational activity, such as progestagens and progestins. In some embodiments, the compressed outer tablet layer comprises one or more progestational agents selected from the group consisting of acetoxypregnenolone, allylestrenol, anagestone acetate, chlormadinone acetate, cyproterone, cyproterone acetate, desogestrel, dihydrogesterone, dimethisterone, ethisterone, ethynodiol diacetate, fluorogestone acetate, gestodene, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, progesterone, dienogest, drospirenone, nomegestrol acetate, hydroxyprogesterone, and trimegestone. In some embodiments, the compressed outer tablet layer comprises one or more progestational agents selected from the group consisting of medroxyprogesterone acetate or trimegestone. In some embodiments, the compressed outer tablet layer comprises medroxyprogesterone acetate. In some embodiments, the compressed outer tablet layer comprises combinations of progestational agents.

As used herein, the term “selective estrogen receptor modulator” is a pharmacological agent with an affinity for the estrogen receptor, which in some tissues acts like an estrogen but block estrogen action in other tissues. In some embodiments, the compressed outer tablet layer comprises one or more selective estrogen receptor modulators selected from the group consisting of TSE-424, ERA-923, raloxifene, tamoxifen, droloxifene, arzoxifene tamoxifen, raloxifene, toremifen, trioxifene, keoxifene, 4-hydroxytamoxifene, clomifene, nafoxidine, dihydroraloxifene, lasofoxifene, and bazedoxifene; or pharmaceutically acceptable salt thereof. In some embodiments, the compressed outer tablet layer comprises one or more selective estrogen receptor modulators selected from the group consisting of those of U.S. Pat. Nos. 5,998,402 and 6,479,535, each of which is hereby incorporated by reference in its entirety. In some embodiments, the compressed outer tablet layer comprises one or more selective estrogen receptor modulators selected from the group consisting of TSE-424, ERA-923, raloxifene, tamoxifen, droloxifene, arzoxifene, and bazedoxifene; or a pharmaceutically acceptable salt thereof. In some embodiments, the compressed outer tablet layer comprises one or more selective estrogen receptor modulators selected from the group consisting of raloxifene and bazedoxifene; or a pharmaceutically acceptable salt thereof. In some embodiments, the compressed outer tablet layer comprises bazedoxifene, or a pharmaceutically acceptable salt thereof. In some embodiments, the compressed outer tablet layer comprises bazedoxifene acetate (bazedoxifene acetic acid salt; “BZA”). In some embodiments, the compressed outer tablet layer comprises combinations of selective estrogen receptor modulators.

U.S. Pat. Nos. 5,998,402 and 6,479,535 report the preparation of bazedoxifene, acetic acid salt (bazedoxifene acetate; “BZA”) and characterize the salt as having a melting point of 174-178° C. The synthetic preparation of bazedoxifene acetate has also appeared in the general literature. See, for example, Miller et al., J. Med. Chem., 2001, 44, 1654-1657, incorporated herein by reference in its entirety, which reports the salt as a crystalline solid having a melting point of 170.5-172.5° C. Further description of the drug's biological activity has appeared in the general literature as well (e.g. Miller, et al. Drugs of the Future, 2002, 27(2), 117-121), incorporated herein by reference in its entirety.

In some embodiments:
the core tablet comprises at least one conjugated estrogen; and
the one or more therapeutic agents are selected from the group consisting of medroxyprogestrone acetate and bazedoxifene acetate.

The estrogen and therapeutic agents can also include pharmaceutically acceptable salts. In some embodiments, the estrogen comprises up to about 20%, up to about 15%, up to about 10%, up to about 9%, up to about 8%, up to about 7%, up to about 6%, up to about 5%, up to about 4%, up to about 3%, up to about 2%, up to about 1%, or up to about 0.5% by weight of the core tablet. In some embodiments, the estrogen comprises from about 0.01 to about 1% by weight of the core tablet.

In some embodiments, the one or more therapeutic agents comprise up to about 20%, up to about 15%, up to about 10%, up to about 9%, up to about 8%, up to about 7%, up to about 6%, up to about 5%, up to about 4%, up to about 3%, up to about 2%, up to about 1%, or up to about 1% by weight of the compressed outer tablet layer. In some embodiments, the one or more therapeutic agents comprise from about 0.1% to about 1% by weight of the compressed outer tablet layer. In some embodiments, the one or more therapeutic agents comprise from about 0.4% to about 0.8% by weight of the compressed outer tablet layer. In some embodiments, the one or more therapeutic agents comprises from about 7% to about 8% by weight of the compressed outer tablet layer.

In some embodiments:
the estrogen comprises from about 0.01% to about 2% by weight of the core tablet; and
the one or more therapeutic agents comprise from about 0.01% to about 10% by weight of the compressed outer tablet layer.

In some embodiments, the core tablet comprises from about 10% to about 70%, from about 10% to about 60%, from about 10% to about 50%, or from about 20% to about 40% by weight of the composition. In some embodiments, the compressed outer tablet layer comprises from about 30% to about 90%, 40% to about 90%, 50% to about 90%, 40% to about 80%, 50% to about 80%, or 60% to about 80% by weight of the composition.

In some embodiments:
the core tablet comprises from about 10% to about 50% by weight of the composition; and
the compressed outer tablet layer comprises from about 50% to about 90% by weight of the composition.

In some embodiments:
the core tablet comprises from about 10% to about 40% by weight of the composition; and
the compressed outer tablet layer comprises from about 60% to about 90% by weight of the composition.

In some embodiments, the compressed outer tablet layer has a hardness from about 2 kp to about 7 kp. In some embodiments, the compressed outer layer does not comprise a surfactant or wetting agent.

In some embodiments, the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, and glycerides of fatty acids. In some embodiments, the compressed outer tablet layer does not comprise sodium lauryl sulfate.

In some embodiments, the compressed outer tablet layer does not comprise a material selected from the group consisting of hydroxyethyl cellulose (HEC) and hydroxypropyl cellulose (HPC). In some embodiments, the compressed outer tablet layer does not comprise a hydroxyalkyl cellulose. In some embodiments, the compressed outer layer comprises at least 10% of the filler/binder component.

In some embodiments:
the core tablet comprises from about 10% to about 50% by weight of the composition;
the compressed outer tablet layer comprises from about 50% to about 90% by weight of the composition;
the compressed outer tablet layer has a hardness from about 2 kp to about 7 kp; and
the compressed outer tablet layer does not comprise a surfactant or wetting agent.

In some embodiments:
the core tablet comprises from about 10% to about 50% by weight of the composition;
the compressed outer tablet layer comprises from about 50% to about 90% by weight of the composition;
the compressed outer tablet layer has a hardness from about 2 kp to about 7 kp; and
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, hydroxyethylcellulose, and hydroxypropylcellulose.

In some embodiments:
the core tablet comprises from about 10% to about 50% by weight of the composition;
the compressed outer tablet layer comprises from about 50% to about 90% by weight of the composition;
the compressed outer tablet layer has a hardness from about 2 kp to about 7 kp;
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, hydroxyethylcellulose, and hydroxypropylcellulose; and
the compressed outer layer comprises at least 10% of the filler/binder component.

In some embodiments:
the core tablet comprises from about 10% to about 50% by weight of the composition;
the compressed outer tablet layer comprises from about 50% to about 90% by weight of the composition;
the compressed outer tablet layer has a hardness from about 2 kp to about 7 kp; and
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, and hydroxyalkylcellulose.

In some embodiments:
the core tablet comprises from about 10% to about 50% by weight of the composition;
the compressed outer tablet layer comprises from about 50% to about 90% by weight of the composition;
the compressed outer tablet layer has a hardness from about 2 kp to about 7 kp;
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, and hydroxyalkylcellulose; and
the compressed outer layer comprises at least 10% of the filler/binder component.

In some embodiments:
the core tablet comprises from about 10% to about 40% by weight of the composition;
the compressed outer tablet layer comprises from about 60% to about 90% by weight of the composition;
the compressed outer tablet layer has a hardness from about 2 kp to about 7 kp; and
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, hydroxyethylcellulose, and hydroxypropylcellulose.

In some embodiments:
the core tablet comprises from about 10% to about 40% by weight of the composition;
the compressed outer tablet layer comprises from about 60% to about 90% by weight of the composition;
the compressed outer tablet layer has a hardness from about 2 kp to about 7 kp; and
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, hydroxyethylcellulose, and hydroxypropylcellulose; and
the compressed outer layer comprises at least 10% of the filler/binder component.

In some embodiments:
the core tablet comprises from about 10% to about 40% by weight of the composition;
the compressed outer tablet layer comprises from about 60% to about 90% by weight of the composition;
the compressed outer tablet layer has a hardness from about 2 kp to about 7 kp; and
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, and hydroxyalkylcellulose.

In some embodiments:
the core tablet comprises from about 10% to about 40% by weight of the composition;
the compressed outer tablet layer comprises from about 60% to about 90% by weight of the composition;
the compressed outer tablet layer has a hardness from about 2 kp to about 7 kp;
the compressed outer tablet layer does not comprise any material selected from the group consisting of sucrose palmitate, Poloxamer 188, metal alkyl sulfate, sodium lauryl sulfate, polyethylene oxide sorbitan fatty acid esters, polyethylene glycol, polyethylene oxide castor oil derivatives, docusate sodium, quaternary ammonium amine compounds, sugar esters of fatty acids, glycerides of fatty acids, and hydroxyalkylcellulose; and
the compressed outer layer comprises at least 10% of the filler/binder component.

In some embodiments, the hydrophilic gel-forming polymer swells in a pH independent manner. In some embodiments, one or both of the core and outer layer hydrophilic gel-forming polymer components comprises one or more of hydroxypropylmethylcellulose, polyethylene oxide, hydroxypropylcellulose, hydroxyethylcellulose, methylcellulose, polyvinylpyrrolidone, xanthan gum, and guar gum. In some embodiments, the hydrophilic gel-forming polymer component is hydroxypropylmethylcellulose (“HPMC”; also known as hypromellose). Suitable HPMC polymers include, but are not limited to the METHOCEL™ line of hydroxypropylmethylcellulose polymers such as METHOCEL™ Premium K100M CR, METHOCEL™ Premium K4M CR, and METHOCEL™ Premium K100 LV (Dow Chemical Co., Midland, Mich.). In some embodiments, the hydrophilic gel-forming polymer component comprises HPMC K100M CR. These embodiments also can be provided for the core and the optional outer layer hydrophilic gel-forming polymer component of the second aspect of the invention.

In some embodiments, one or both of the core and outer layer hydrophilic gel-forming polymer components comprises a hydroxypropylmethylcellulose polymer having from about 7% to about 12% by weight hydroxypropoxyl groups. In some embodiments, one or both of the core and outer layer hydrophilic gel-forming polymer components comprises a hydroxypropylmethylcellulose polymer having from about 19% to about 24% by weight methoxyl groups. These embodiments also can be provided for the core and the optional outer layer hydrophilic gel-forming polymer component of the second aspect of the invention.

In some embodiments, one or both of the core and outer layer hydrophilic gel-forming polymer components comprises a polymer having an apparent viscosity from about 80 cP to about 150,000 cP. In some embodiments, one or both of the core and outer layer hydrophilic gel-forming polymer components comprises a polymer having an apparent viscosity from about 3000 to about 6000 cP. In some embodiments, one or both of the core and outer layer hydrophilic gel-forming polymer components comprises a polymer having an apparent viscosity from about 80 to about 120 cP. In some embodiments, one or both of the core and outer layer hydrophilic gel-forming polymer components comprises a polymer having an apparent viscosity from about 80,000 to about 120,000 cP. The previously described embodiments also can be provided for the core and the optional outer layer hydrophilic gel-forming polymer component of the second aspect of the invention.

In some embodiments, one or both of the core and outer layer filler/diluent components comprises one or more filler substances. In some embodiments, one or both of the core and outer layer filler/diluent components comprises one or more diluent substances. In some embodiments, one or both of the core and outer layer filler/diluent components comprises one or more substances that are diluents and fillers.

In some embodiments, the core filler/diluent component of the first, second, or third aspect of the invention comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate. In some embodiments, the core filler/diluent component of the first, second, or third aspect of the invention comprises one or more of lactose, lactose monohydrate, mannitol, sucrose, maltodextrin, sorbitol, and xylitol. In some embodiments, the core filler/diluent component of the first, second, or third aspect of the invention comprises one or more of lactose and lactose monohydrate. In some embodiments, the core filler/diluent component of the first or second aspect of the invention does not comprise sucrose.

In some embodiments, the outer layer filler/diluent component of the first or third aspect of the invention or the optional outer layer filler/diluent component of the second aspect of the invention, if present, comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate. In some embodiments, the outer layer filler/diluent component of the first or third aspect of the invention or the optional outer layer filler/diluent component of the second aspect of the invention, if present, comprises one or more of lactose, lactose monohydrate, mannitol, sucrose, maltodextrin, sorbitol, and xylitol. In some embodiments, the outer layer filler/diluent component of the first or third aspect of the invention or the optional outer layer filler/diluent component of the second aspect of the invention, if present, comprises one or more of lactose and lactose monohydrate. In some embodiments, the outer layer filler/diluent component of the first or third aspect of the invention or the optional outer layer filler/diluent component of the second aspect of the invention, if present, does not comprise sucrose.

In some embodiments, the term “binder” refers to a substance that increases the mechanical strength and/or compressibility of a pharmaceutical composition comprising the pharmaceutical formulations of the invention. In some embodiments, one or both of the core and outer layer filler/binder components comprises one or more filler substances. In some embodiments, one or both of the core and outer layer filler/binder components comprises one or more binder substances. In some embodiments, one or both of the core and outer layer filler/binder components comprises one or more substances that are fillers and binders.

In some embodiments, the core filler/binder component of the first, second, or third aspect of the invention comprises one or more of microcrystalline cellulose, polyvinylpyrrolidone, copovidone, polyvinylalcohol, starch, gelatin, gum arabic, gum acacia, and gum tragacanth. In some embodiments, the core filler/binder component of the first, second, or third aspect of the invention comprises microcrystalline cellulose.

In some embodiments, the outer layer filler/binder component of the first aspect of the invention or the optional outer layer filler/binder component of the second aspect of the invention, if present, comprises one or more of microcrystalline cellulose, polyvinylpyrrolidone, copovidone, polyvinylalcohol, starch, gelatin, gum arabic, gum acacia, and gum tragacanth. In some embodiments, the outer layer filler/binder component of the first aspect of the invention or the optional outer layer filler/binder component of the second aspect of the invention, if present, comprises microcrystalline cellulose. In some embodiments, the outer layer filler/binder component of the first aspect of the invention or the optional outer layer filler/binder component of the second aspect of the invention, if present, does not comprise polyvinylpyrrolidone.

In some embodiments, the outer layer filler/binder component of the third aspect of the invention comprises one or more of silicified microcrystalline cellulose, microcrystalline cellulose, polyvinylpyrrolidone, copovidone, polyvinylalcohol, starch, gelatin, gum arabic, gum acacia, and gum tragacanth. In some embodiments, the outer layer filler/binder component of the third aspect of the invention comprises silicified microcrystalline cellulose. In some embodiments, the outer layer filler/binder component of the third aspect of the invention does not comprise polyvinylpyrrolidone.

In some embodiments, one or both of the core tablet and the compressed outer tablet layer optionally comprises a lubricant component. In some embodiments, the optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol. In some embodiments, the optional core lubricant component, if present, comprises one or more of stearic acid, metallic stearate, sodium stearyl fumarate, glyceryl behenate, mineral oil, vegetable oil, and paraffin. In some embodiments, the optional core lubricant component, if present, comprises magnesium stearate.

In some embodiments, the optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol. In some embodiments, the optional outer layer lubricant component, if present, comprises one or more of stearic acid, metallic stearate, sodium stearyl fumarate, glyceryl behenate, mineral oil, vegetable oil, and paraffin. In some embodiments, the optional outer layer lubricant component, if present, comprises magnesium stearate. The previously described embodiments can also be provided for the first, second, or third aspect of the invention.

In some embodiments, the compressed outer tablet layer optionally comprises an antioxidant component. The antioxidant component can be a single compound, such as ascorbic acid, or a mixture of antioxidants. A wide variety of antioxidant compound are known in the art, and are suitable for use in the present invention. Examples of such antioxidants that can be used in the present invention include vitamin E, vitamin E acetate (for example, dry vitamin E acetate 50% DC from BASF, Florham Park, N.J.; also known as D,L-α-tocopheryl acetate) sodium ascorbate, ascorbyl palmitate, BHT (butylated hydroxytoluene) and BHA (butylated hydroxyanisole), each optionally in conjunction with an amount of ascorbic acid.

In some embodiments, the antioxidant component, if present, comprises one or more of ascorbic acid, sodium ascorbate, ascorbyl palmitate, vitamin E, vitamin E acetate, butylated hydroxytoluene, and butylated hydroxyanisole. In some embodiments, the optional antioxidant component, if present, comprises one or more of ascorbic acid, vitamin E, and vitamin E acetate. In some embodiments, the optional antioxidant component, if present, comprises one or more of ascorbic acid and vitamin E acetate. The previously described embodiments can also be provided for the first, second, or third aspect of the invention.

In some embodiments, the outer layer disintegrant component of the third aspect of the invention comprises one or more of croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose. In some embodiments, the outer layer disintegrant component of the third aspect of the invention comprises one or more of sodium starch glycolate and pregelatinized starch.

In some embodiments, the optional wetting agent component of the third aspect of the invention, if present, comprises one or more of a polyethylene glycol-polypropylene glycol copolymer, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, polyethoxylated fatty acid esters, or polyglycolized glycerides. In some embodiments, the optional wetting agent component of the third aspect of the invention, if present, comprises a polyethylene glycol-polypropylene glycol copolymer. In some embodiments, the optional wetting agent component of the third aspect of the invention, if present, comprises Poloxamer 188.

In some embodiments, the core hydrophilic gel-forming polymer component of the first or second aspect of the invention comprises from about 1% to about 40%, from about 1% to about 30%, from about 5% to about 15%, from about 15% to about 25%, from about 25% to about 35%, or from about 30% to about 40% by weight of the core tablet. In some embodiments, the outer layer hydrophilic gel-forming polymer component of the first aspect of the invention or the optional outer layer hydrophilic gel-forming polymer component of the second aspect of the invention, if present, comprises from about 1% to about 60%, from about 1% to about 50%, from about 1% to about 40%, from about 1% to about 30%, from about 1% to about 8%, from about 8% to about 15%, from about 15% to about 30%, from about 30% to about 50%, from about 50% to about 60%, or from about 30% to about 60% by weight of the compressed outer tablet layer.

In some embodiments, the core filler/diluent component of the first, second, or third aspect of the invention comprises from about 30% to about 85%, from about 40% to about 85%, from about 40% to about 75%, from about 50% to about 85%, from about 50% to about 60%, from about 60% to about 70% or from about 70% to about 80% by weight of the core tablet. In some embodiments, the outer layer filler/diluent component of the first aspect of the invention or the optional outer layer filler/diluent component of the second aspect of the invention, if present, comprises from about 10% to about 80%, from about 10% to about 70%, from about 10% to about 60%, from about 10% to about 50%, from about 10% to about 40%, from about 10% to about 20%, from about 20% to about 30%, from about 30% to about 40%, from about 40% to about 50%, from about 50% to about 60%, from about 60% to about 70%, from about 20% to about 60%, or from about 30% to about 60% by weight of the compressed outer tablet layer. In some embodiments, the outer layer filler/diluent component of the third aspect of the invention comprises from about 25% to about 65%, from about 35% to about 55%, or from about 40% to about 50% of the compressed outer tablet layer.

In some embodiments, the core filler/binder component of the first, second, or third aspect of the invention comprises from about 1% to about 30%, from about 5% to about 25%, from about 10% to about 20% by weight of the core tablet. In some embodiments, the outer layer filler/binder component of the first aspect of the invention or the optional outer layer filler/binder component of the second aspect of the invention, if present, comprises from about 1% to about 70%, from about 1% to about 60%, from about 1% to about 50%, from about 1% to about 10%, from about 10% to about 30%, from about 30% to about 40%, from about 40% to about 50%, or from about 50% to about 60% by weight of the compressed outer tablet layer. In some embodiments, the outer layer filler/diluent component of the third aspect of the invention comprises from about 20% to about 50%, from about 25% to about 45%, or from about 30% to about 40% of the compressed outer tablet layer.

In some embodiments, the optional core lubricant component, if present, comprises from about 0.01% to about 2%, from about 0.01% to about 1%, from about 0.1% to about 2%, or from about 0.1% to about 1% of a lubricant component by weight of the core tablet. In some embodiments, the optional outer layer lubricant component, if present, comprises from about 0.01% to about 2%, 0.01% to about 1%, 0.1% to about 2%, or about 0.1% to about 1% of a lubricant component by weight of the compressed outer tablet layer.

In some embodiments, the optional antioxidant, if present, comprises from about 0.01% to about 4%, from about 0.01% to about 3%, or from about 0.01% to about 2% of an antioxidant component by weight of the compressed outer tablet layer.

In some embodiments, the outer layer disintegrant component of the third aspect of the invention comprises from about 2% to about 15%, from about 5% to about 15%, from about 8% to about 12%, or from about 9% to about 11%, of the compressed outer tablet layer.

In some embodiments, the optional outer layer wetting agent component of the third aspect of the invention, if present, comprises from about 0.01% to about 4%, from about 0.1% to about 3%, from about 0.1% to about 3%, from about 0.5% to about 3%, or from about 1% to about 3% of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet; and
the outer layer hydrophilic gel-forming polymer component comprises from about 1% to about 8% by weight of the compressed outer tablet layer.

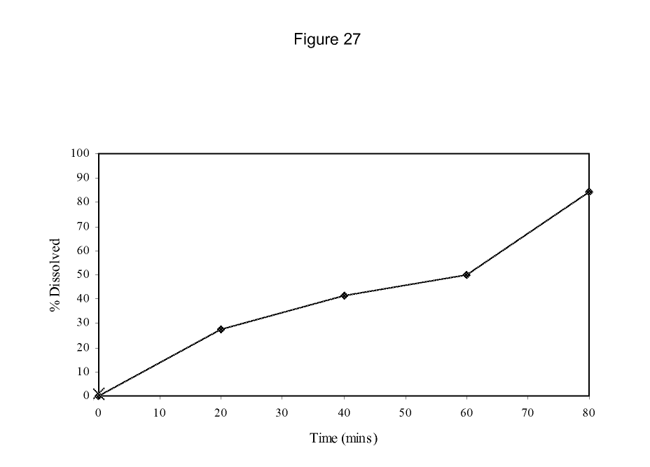

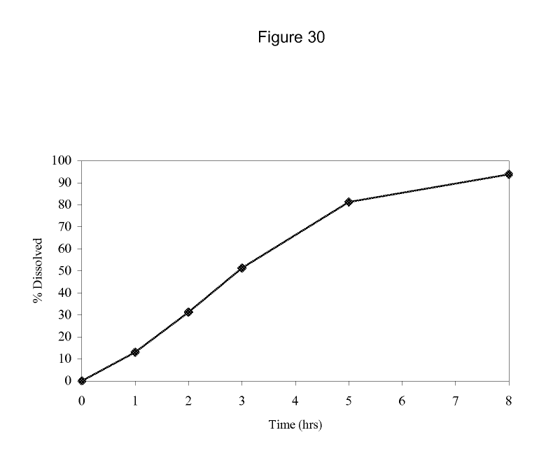

In some embodiments of the previous embodiment:
the core tablet comprises at least one conjugated estrogen;
the compressed outer tablet layer comprises bazedoxifene acetate;
the dissolution profile of the estrogen from the composition is substantially as shown in any one of FIG. 30, 48, 51, or 53; and
the dissolution profile of the therapeutic agent from the composition under type II therapeutic agent dissolution conditions is substantially as shown in any one of FIG. 27, 41, 44, or 46.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet; and
the outer layer hydrophilic gel-forming polymer component comprises from about 8% to about 15% by weight of the compressed outer tablet layer.

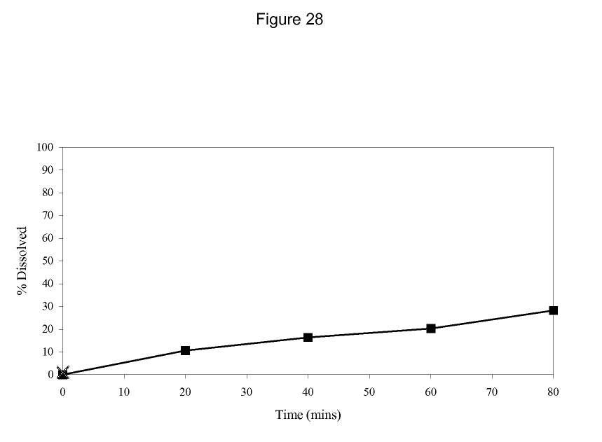

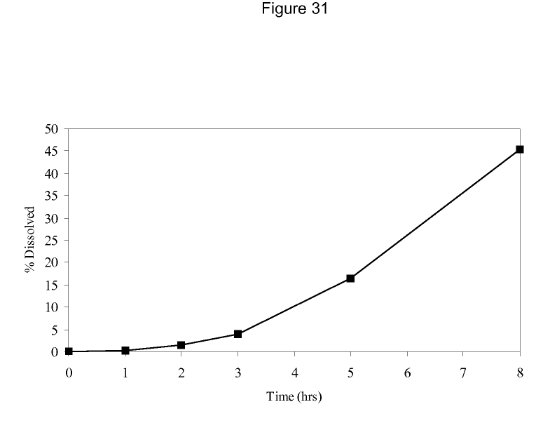

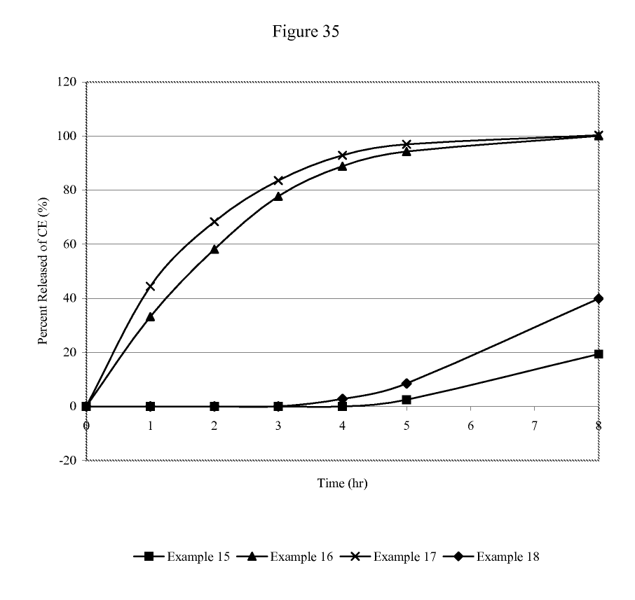

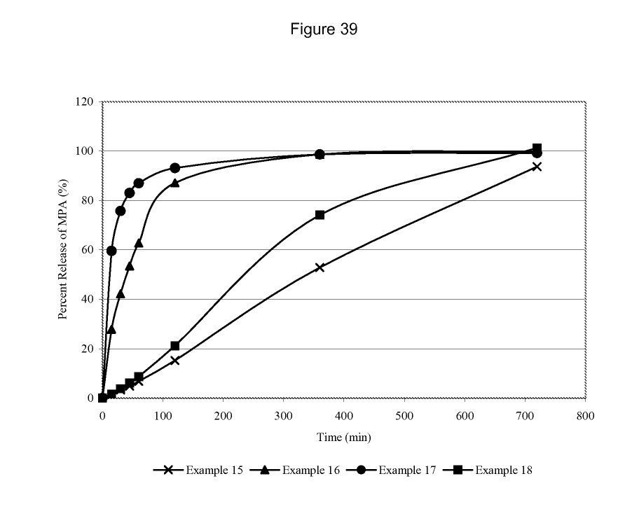

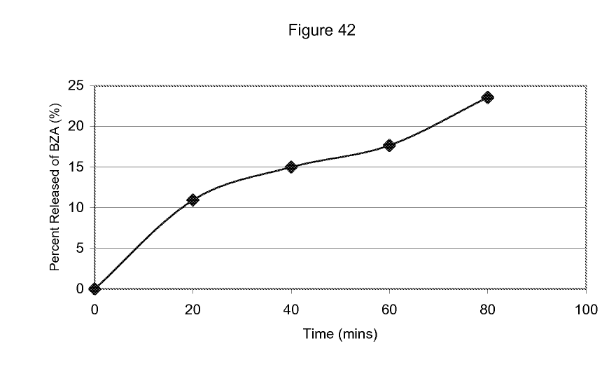

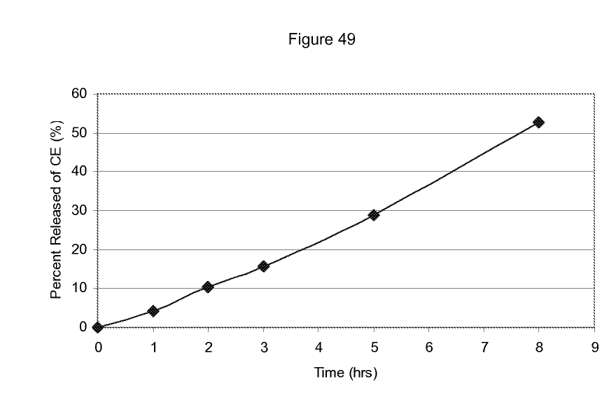

In some embodiments of the previous embodiment:
the core tablet comprises at least one conjugated estrogen;
the compressed outer tablet layer comprises bazedoxifene acetate;
the dissolution profile of the estrogen from the composition is substantially as shown in FIG. 31 or 49; and
the dissolution profile of the therapeutic agent from the composition under type II therapeutic agent dissolution conditions is substantially as shown in FIG. 28 or 42; or
the core tablet comprises at least one conjugated estrogen;
the compressed outer tablet layer comprises medroxyprogesterone acetate;
the dissolution profile of the estrogen from the composition is substantially as shown in FIG. 35 for Example 16; and
the dissolution profile of the therapeutic agent from the composition under type I therapeutic agent dissolution conditions is substantially as shown in FIG. 39 for Example 16.

In some embodiments:
the core filler/diluent component comprises from about 50% to about 85% by weight the core tablet;
the core filler/binder component comprises from about 10% to about 20% by weight of the core tablet;
the core hydrophilic gel-forming polymer component comprises from about 5% to about 15% by weight of the core tablet; and
the outer layer hydrophilic gel-forming polymer component comprises from about 15% to about 30% by weight of the compressed outer tablet layer.

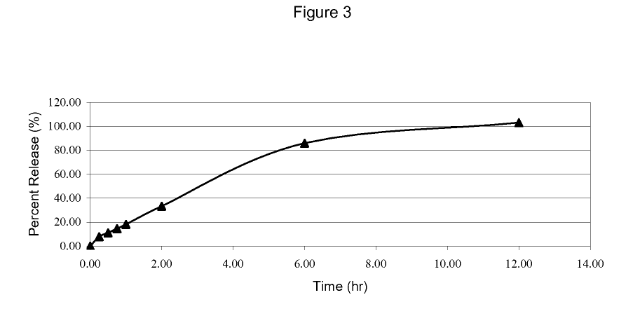

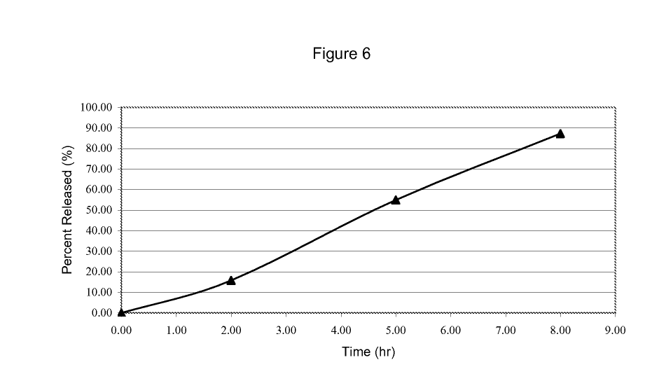

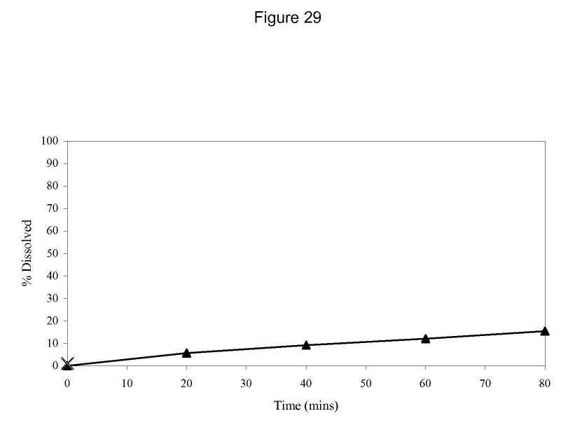

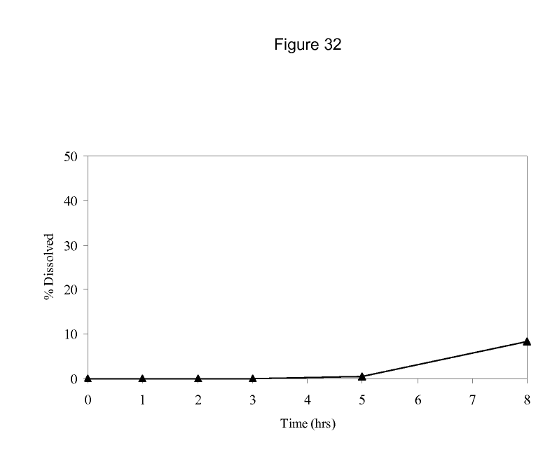

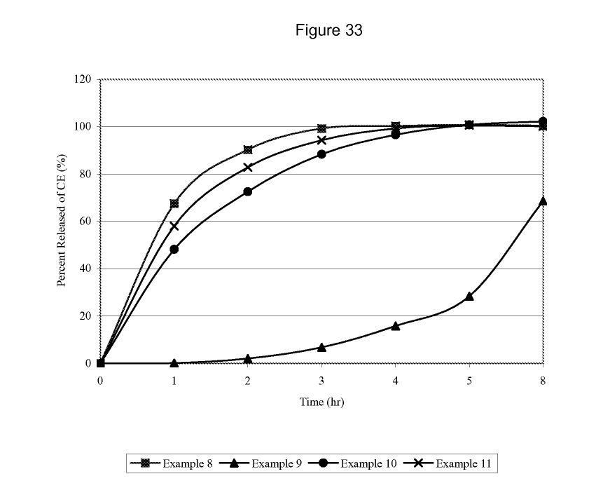

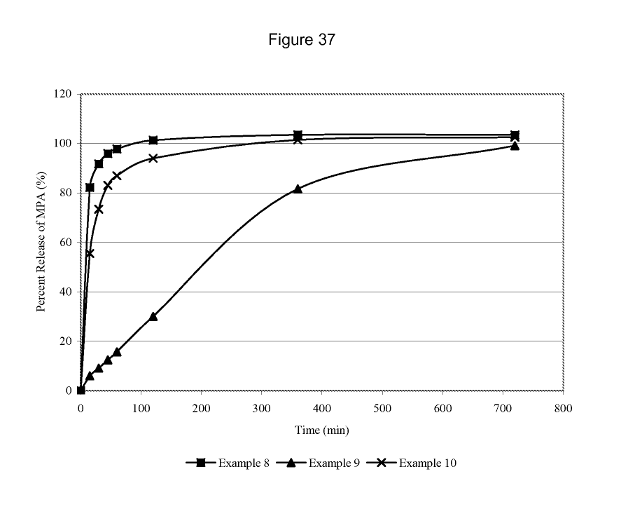

In some embodiments of the previous embodiment:
the core tablet comprises at least one conjugated estrogen;
the compressed outer tablet layer comprises bazedoxifene acetate;
the dissolution profile of the estrogen from the composition is substantially as shown in any one of FIG. 32, 50, 52, or 54; and
the dissolution profile of the therapeutic agent from the composition under type II therapeutic agent dissolution conditions is substantially as shown in any one of FIG. 29, 43, 45, or 47; or
the core tablet comprises at least one conjugated estrogen;
the compressed outer tablet layer comprises medroxyprogesterone acetate;
the dissolution profile of the estrogen from the composition is substantially as shown in any one of FIG. 6, FIG. 33 for Example 9, or FIG. 35 for Example 18; and
the dissolution profile of the therapeutic agent from the composition under type I therapeutic agent dissolution conditions is substantially as shown in any one of FIG. 3, FIG. 37 for Example 9, or FIG. 39 for Example 18.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet; and
the outer layer hydrophilic gel-forming polymer component comprises from about 30% to about 50% by weight of the compressed outer tablet layer.

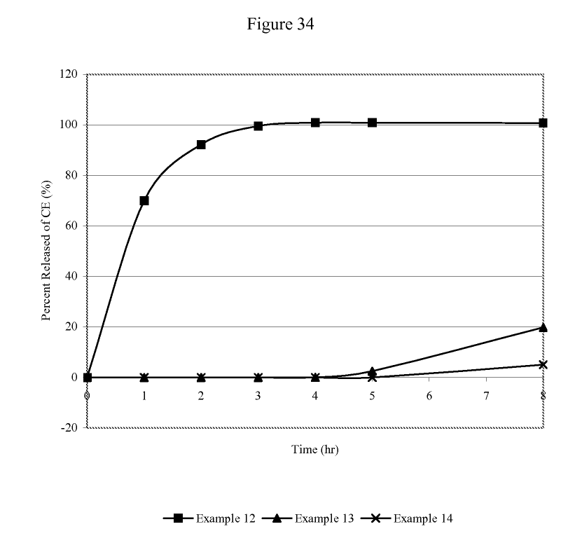

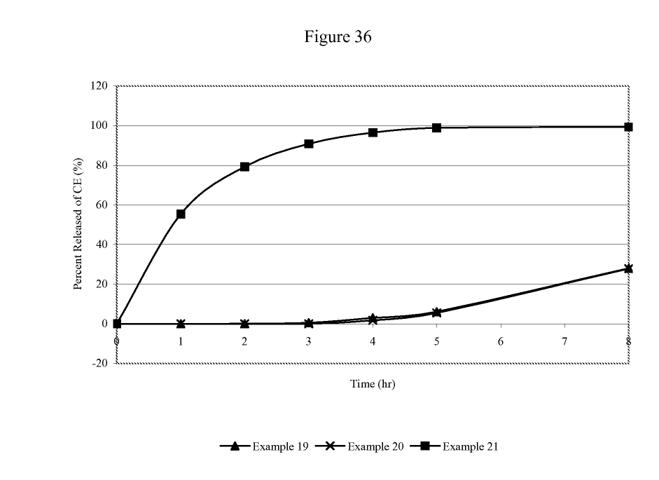

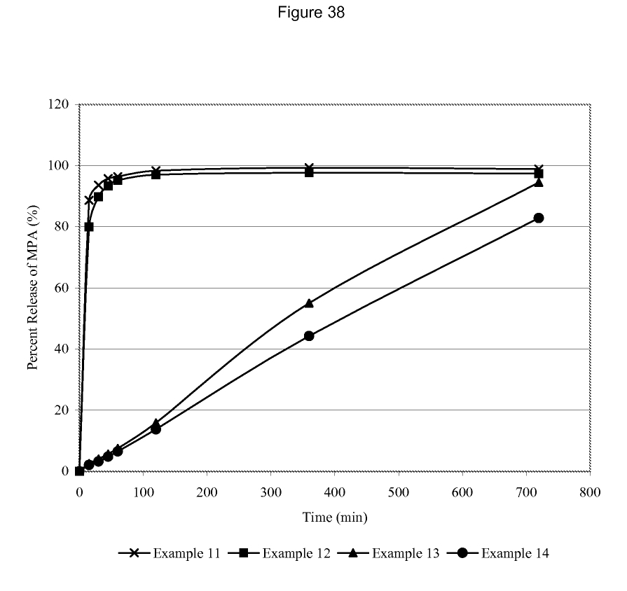

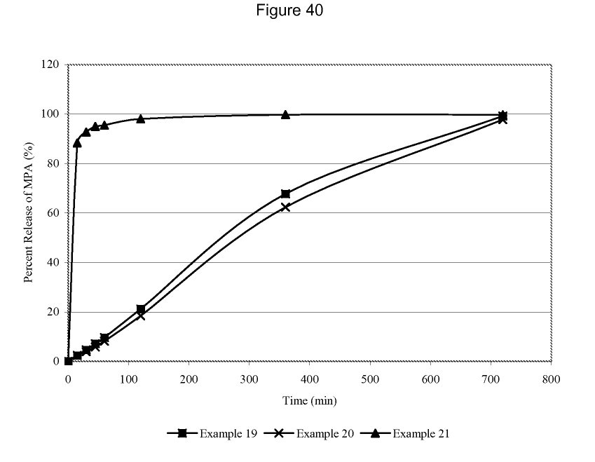

In some embodiments of the previous embodiment:
the core tablet comprises at least one conjugated estrogen;
the compressed outer tablet layer comprises medroxyprogesterone acetate;
the dissolution profile of the estrogen from the composition is substantially as shown in any one of FIG. 36 for Example 20, FIG. 35 for Example 15, or FIG. 34 for Example 13; and
the dissolution profile of the therapeutic agent from the composition under type I therapeutic agent dissolution conditions is substantially as shown in any one of FIG. 40 for Example 20, FIG. 39 for Example 15, or FIG. 38 for Example 13.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the outer layer hydrophilic gel-forming polymer component comprises from about 1% to about 8% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the outer layer hydrophilic gel-forming polymer component comprises from about 8% to about 15% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the outer layer hydrophilic gel-forming polymer component comprises from about 15% to about 30% by weight of the compressed outer tablet layer.

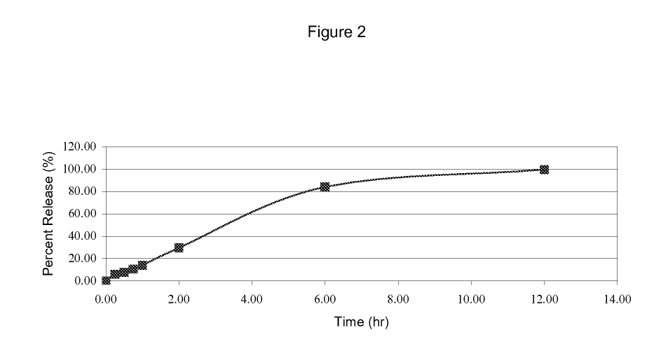

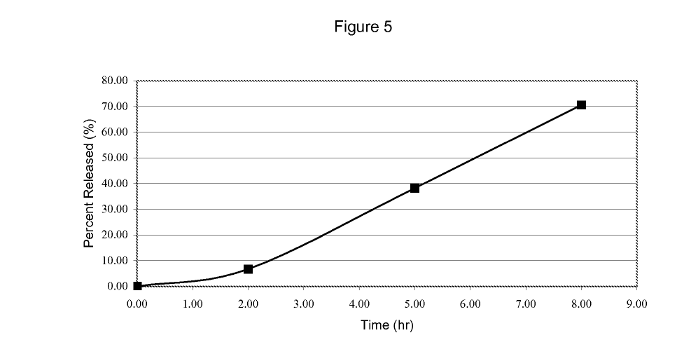

In some embodiments of the previous embodiment:
the core tablet comprises at least one conjugated estrogen;
the compressed outer tablet layer comprises medroxyprogesterone acetate;
the dissolution profile of the estrogen from the composition is substantially as shown in FIG. 5; and
the dissolution profile of the therapeutic agent from the composition under type I therapeutic agent dissolution conditions is substantially as shown in FIG. 2.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the outer layer hydrophilic gel-forming polymer component comprises from about 30% to about 50% by weight of the compressed outer tablet layer.

In some embodiments:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the outer layer hydrophilic gel-forming polymer component comprises from about 1% to about 8% by weight of the compressed outer tablet layer.

In some embodiments:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the outer layer hydrophilic gel-forming polymer component comprises from about 8% to about 15% by weight of the compressed outer tablet layer.

In some embodiments:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the outer layer hydrophilic gel-forming polymer component comprises from about 15% to about 30% by weight of the compressed outer tablet layer.

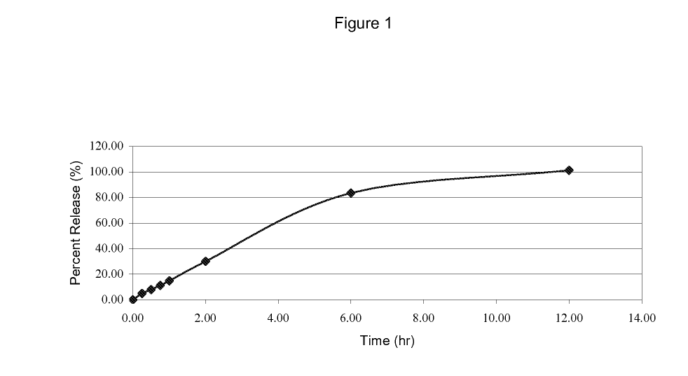

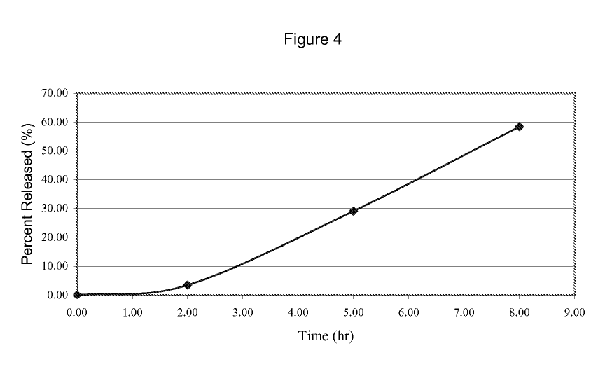

In some embodiments of the previous embodiment:
the core tablet comprises at least one conjugated estrogen;
the compressed outer tablet layer comprises medroxyprogesterone acetate;
the dissolution profile of the estrogen from the composition is substantially as shown in FIG. 4; and
the dissolution profile of the therapeutic agent from the composition under type I therapeutic agent dissolution conditions is substantially as shown in FIG. 1.

In some embodiments:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the outer layer hydrophilic gel-forming polymer component comprises from about 30% to about 50% by weight of the compressed outer tablet layer.

In some embodiments:
the core filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the core filler/binder component comprises one or more of microcrystalline cellulose, polyvinylpyrrolidone, copovidone, polyvinylalcohol, starch, gelatin, gum arabic, gum acacia, and gum tragacanth;
the core hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the outer layer filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the outer layer filler/binder component comprises one or more of microcrystalline cellulose, polyvinylpyrrolidone, copovidone, polyvinylalcohol, starch, gelatin, gum arabic, gum acacia, and gum tragacanth;
the outer layer hydrophilic gel-forming polymer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional antioxidant component, if present, comprises one or more of ascorbic acid, sodium ascorbate, ascorbyl palmitate, vitamin E, vitamin E acetate, butylated hydroxytoluene, and butylated hydroxyanisole;
the core tablet comprises at least one conjugated estrogen; and
the compressed outer tablet layer comprises medroxyprogesterone acetate or bazedoxifene acetate.

In some embodiments:
the core filler/diluent component comprises one or more of lactose and lactose monohydrate;
the core filler/binder component comprises microcrystalline cellulose;
the core hydrophilic gel-forming polymer component comprises hydroxypropylmethylcellulose;
the optional core lubricant component, if present, comprises magnesium stearate;
the outer layer filler/diluent component comprises one or more of lactose and lactose monohydrate;
the outer layer filler/binder component comprises microcrystalline cellulose;
the outer layer hydrophilic gel-forming polymer comprises hydroxypropylmethylcellulose;
the optional outer layer lubricant component, if present, comprises magnesium stearate;
the optional antioxidant component, if present, comprises one or more of ascorbic acid and vitamin E acetate;
the core tablet comprises at least one conjugated estrogen; and
the compressed outer tablet layer comprises medroxyprogesterone acetate or bazedoxifene acetate.

In some embodiments, the pharmaceutically acceptable carrier component in the second aspect of the invention comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, a metal carbonate, polyvinylpyrrolidone, copovidone, polyvinylalcohol, gelatin, gum arabic, gum acacia, gum tragacanth, hydroxypropylmethylcellulose, polyethylene oxide, hydroxypropylcellulose, hydroxyethylcellulose, methylcellulose, polyvinylpyrrolidone, xanthan gum, and guar gum. In some embodiments, the pharmaceutically acceptable carrier component comprises one or more of lactose, lactose monohydrate, microcrystalline cellulose, and hydroxypropylmethylcellulose.

In some embodiments, the pharmaceutically acceptable carrier component comprises an outer layer filler/diluent component. In some embodiments, the pharmaceutically acceptable carrier component comprises an outer layer filler/binder component. In some embodiments, the pharmaceutically acceptable carrier component comprises an outer layer hydrophilic gel-forming polymer component.

In some embodiments, the pharmaceutically acceptable carrier component comprises:
from about 30% to about 99.9% by weight of an outer layer filler/diluent component; and
from about 1% to about 70% by weight of an outer layer filler/binder component.

In some embodiments, the pharmaceutically acceptable carrier component comprises:
from about 30% to about 99.9% by weight of an outer layer filler/diluent component; and
from about 1% to about 70% by weight of an outer layer hydrophilic gel-forming polymer component.

In some embodiments, the pharmaceutically acceptable carrier component comprises:
from about 30% to about 99.9% by weight of an outer layer filler/binder component; and
from about 1% to about 70% by weight of an outer layer hydrophilic gel-forming polymer component.

In some embodiments, the pharmaceutically acceptable carrier component comprises:
from about 50% to about 99% by weight of an outer layer filler/diluent component; and
from about 1% to about 30% by weight of an outer layer filler/binder component.

In some embodiments, the pharmaceutically acceptable carrier component comprises:
from about 50% to about 99% by weight of an outer layer filler/diluent component; and
from about 1% to about 30% by weight of an outer layer hydrophilic gel-forming polymer component.

In some embodiments, the pharmaceutically acceptable carrier component comprises:
from about 50% to about 99% by weight of an outer layer filler/binder component; and
from about 1% to about 30% by weight of an outer layer hydrophilic gel-forming polymer component.

In some embodiments, the pharmaceutically acceptable carrier component comprises:
from about 20% to about 60% by weight of an outer layer filler/diluent component; and
from about 20% to about 60% by weight of an outer layer filler/binder component.

In some embodiments, the pharmaceutically acceptable carrier component comprises:
from about 20% to about 60% by weight of an outer layer filler/diluent component; and
from about 20% to about 60% by weight of an outer layer hydrophilic gel-forming polymer component.

In some embodiments, the pharmaceutically acceptable carrier component comprises:
from about 20% to about 60% by weight of an outer layer filler/binder component; and
from about 20% to about 60% by weight of an outer layer hydrophilic gel-forming polymer component.

In some embodiments:
the pharmaceutically acceptable carrier component comprises an outer layer filler/binder component;
the core tablet comprises at least one conjugated estrogen;
the compressed outer tablet layer comprises medroxyprogesterone acetate;
the dissolution profile of the estrogen from the composition is substantially as shown in FIG. 34 for Example 14; and
the dissolution profile of the therapeutic agent from the composition under type I therapeutic agent dissolution conditions is substantially as shown in FIG. 38 for Example 14.

In some embodiments:
the pharmaceutically acceptable carrier component comprises:
   from about 30% to about 99.9% by weight of an outer layer filler/diluent component; and
   from about 1% to about 70% by weight of an outer layer filler/binder component; and
the core tablet comprises at least one conjugated estrogen;
the compressed outer tablet layer comprises medroxyprogesterone acetate;
the dissolution profile of the estrogen from the composition is substantially as shown in any one of FIG. 33 for Example 11, FIG. 33 for Example 8, FIG. 34 for Example 12, or FIG. 36 for Example 21; and
the dissolution profile of the therapeutic agent from the composition under type I therapeutic agent dissolution conditions is substantially as shown in any one of FIG. 38 for Example 11, FIG. 37 for Example 8, FIG. 38 for Example 12, or FIG. 40 for Example 21.

In some embodiments:
the pharmaceutically acceptable carrier component comprises:
   from about 30% to about 99.9% by weight of an outer layer filler/binder component; and
   from about 1% to about 70% by weight of an outer layer hydrophilic gel-forming polymer component; and
the core tablet comprises at least one conjugated estrogen;
the compressed outer tablet layer comprises medroxyprogesterone acetate;
the dissolution profile of the estrogen from the composition is substantially as shown in FIG. 36 for Example 19 or FIG. 34 for Example 14; and
the dissolution profile of the therapeutic agent from the composition under type I therapeutic agent dissolution conditions is substantially as shown in FIG. 40 for Example 19 or FIG. 38 for Example 14.

In some embodiments:
the pharmaceutically acceptable carrier component comprises an outer layer filler/binder component;
the core tablet comprises at least one conjugated estrogen;
the compressed outer tablet layer comprises medroxyprogesterone acetate;
the dissolution profile of the estrogen from the composition is substantially as shown in FIG. 33 for Example 10 or FIG. 35 for Example 17; and
the dissolution profile of the therapeutic agent from the composition under type I therapeutic agent dissolution conditions is substantially as shown in FIG. 37 for Example 10 or FIG. 39 for Example 17.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet; and
the optional outer layer hydrophilic gel-forming polymer component, if present, comprises from about 1% to about 8% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet; and
the optional outer layer hydrophilic gel-forming polymer component, if present, comprises from about 8% to about 15% by weight of the compressed outer tablet layer.

In some embodiments:
the core filler/diluent component comprises from about 50% to about 85% by weight the core tablet;
the core filler/binder component comprises from about 10% to about 20% by weight of the core tablet;
the core hydrophilic gel-forming polymer component comprises from about 5% to about 15% by weight of the core tablet; and
the optional outer layer hydrophilic gel-forming polymer component, if present, comprises from about 15% to about 30% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet; and
the optional outer layer hydrophilic gel-forming polymer component, if present, comprises from about 30% to about 50% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the optional outer layer hydrophilic gel-forming polymer component, if present, comprises from about 1% to about 8% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the optional outer layer hydrophilic gel-forming polymer component, if present, comprises from about 8% to about 15% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the optional outer layer hydrophilic gel-forming polymer component, if present, comprises from about 15% to about 30% by weight of the compressed outer tablet layer.

In some embodiments:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 25% by weight of the core tablet; and
the optional outer layer hydrophilic gel-forming polymer component, if present, comprises from about 30% to about 50% by weight of the compressed outer tablet layer.

In some embodiments:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the optional outer layer hydrophilic gel-forming polymer component, if present, comprises from about 1% to about 8% by weight of the compressed outer tablet layer.

In some embodiments:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the optional outer layer hydrophilic gel-forming polymer component, if present, comprises from about 8% to about 15% by weight of the compressed outer tablet layer.

In some embodiments:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the optional outer layer hydrophilic gel-forming polymer component, if present, comprises from about 15% to about 30% by weight of the compressed outer tablet layer.

In some embodiments of the second aspect of the invention:
the core filler/diluent component comprises from about 40% to about 75% by weight of the core tablet;
the core filler/binder component comprises from about 10% to about 20% by weight of the core tablet;
the core hydrophilic gel-forming polymer component comprises from about 25% to about 35% by weight of the core tablet; and
the optional outer layer hydrophilic gel-forming polymer component, if present, comprises from about 30% to about 50% by weight of the compressed outer tablet layer.

The core filler/diluent component, the core filler/binder component, the core hydrophilic gel-forming polymer component, the core outer layer lubricant component, the optional outer layer filler/diluent component, the optional outer layer filler/binder component, the optional hydrophilic gel-forming polymer component, and the optional outer layer lubricant component in the embodiments of the second aspect of the invention can comprise the same materials as described herein for the first aspect of the invention.

In some embodiments of the second aspect of the invention:
the core filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the core filler/binder component comprises one or more of microcrystalline cellulose, polyvinylpyrrolidone, copovidone, polyvinylalcohol, starch, gelatin, gum arabic, gum acacia, and gum tragacanth;
the core hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the pharmaceutically acceptable carrier component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, a metal carbonate, polyvinylpyrrolidone, copovidone, polyvinylalcohol, gelatin, gum arabic, gum acacia, gum tragacanth, hydroxypropylmethylcellulose, polyethylene oxide, hydroxypropylcellulose, hydroxyethylcellulose, methylcellulose, polyvinylpyrrolidone, xanthan gum, and guar gum;
the optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional antioxidant component, if present, comprises one or more of ascorbic acid, sodium ascorbate, ascorbyl palmitate, vitamin E, vitamin E acetate, butylated hydroxytoluene, and butylated hydroxyanisole;
the core tablet comprises at least one conjugated estrogen; and
the compressed outer tablet layer comprises medroxyprogesterone acetate or bazedoxifene acetate.

In some embodiments:
the core filler/diluent component comprises one or more of lactose and lactose monohydrate;
the core filler/binder component comprises microcrystalline cellulose;
the core hydrophilic gel-forming polymer component comprises hydroxypropylmethylcellulose;
the optional core lubricant component, if present, comprises magnesium stearate;
the pharmaceutically acceptable carrier component comprises one or more of lactose, lactose monohydrate, microcrystalline cellulose, and hydroxypropylmethylcellulose;
the optional outer layer lubricant component, if present, comprises magnesium stearate;
the optional antioxidant component, if present, comprises one or more of ascorbic acid and vitamin E acetate;
the core tablet comprises at least one conjugated estrogen; and
the compressed outer tablet layer comprises medroxyprogesterone acetate or bazedoxifene acetate.

In some embodiments of the second aspect of the invention:
the core filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the core filler/binder component comprises one or more of microcrystalline cellulose, polyvinylpyrrolidone, copovidone, polyvinylalcohol, starch, gelatin, gum arabic, gum acacia, and gum tragacanth;
the core hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional outer layer filler/diluent component, if present, comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the optional outer layer filler/binder component, if present, comprises one or more of microcrystalline cellulose, polyvinylpyrrolidone, copovidone, polyvinylalcohol, starch, gelatin, gum arabic, gum acacia, and gum tragacanth;
the optional outer layer hydrophilic gel-forming polymer component, if present, comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional antioxidant component, if present, comprises one or more of ascorbic acid, sodium ascorbate, ascorbyl palmitate, vitamin E, vitamin E acetate, butylated hydroxytoluene, and butylated hydroxyanisole;
the core tablet comprises at least one conjugated estrogen; and
the compressed outer tablet layer comprises medroxyprogesterone acetate or bazedoxifene acetate.

In some embodiments of the second aspect of the invention:
the core filler/diluent component comprises one or more of lactose and lactose monohydrate;
the core filler/binder component comprises microcrystalline cellulose;
the core hydrophilic gel-forming polymer component comprises hydroxypropylmethylcellulose;
the optional core lubricant component, if present, comprises magnesium stearate;
the optional outer layer filler/diluent component, if present, comprises one or more of lactose and lactose monohydrate;
the optional outer layer filler/binder component, if present, comprises microcrystalline cellulose;
the optional outer layer hydrophilic gel-forming polymer component, if present, comprises hydroxypropylmethylcellulose;
the optional outer layer lubricant component, if present, comprises magnesium stearate;
the optional antioxidant component, if present, comprises one or more of ascorbic acid and vitamin E acetate;
the core tablet comprises at least one conjugated estrogen; and
the compressed outer tablet layer comprises medroxyprogesterone acetate or bazedoxifene acetate.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet;
the outer layer filler/diluent component comprises from about 35% to about 55% by weight of the compressed outer tablet layer;
the outer layer filler/binder component comprises from about 25% to about 45% by weight of the compressed outer tablet layer;
a disintegrant component comprising from about 5% to about 15% by weight of the compressed outer tablet layer;
optionally, an outer layer wetting agent component comprising from about 0.1% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 30% by weight of the core tablet;
the outer layer filler/diluent component comprises from about 35% to about 55% by weight of the compressed outer tablet layer;
the outer layer filler/binder component comprises from about 25% to about 45% by weight of the compressed outer tablet layer;
a disintegrant component comprising from about 5% to about 15% by weight of the compressed outer tablet layer;
optionally, an outer layer wetting agent component comprising from about 0.1% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 25% to about 35% by weight of the core tablet;
the outer layer filler/diluent component comprises from about 35% to about 55% by weight of the compressed outer tablet layer;
the outer layer filler/binder component comprises from about 25% to about 45% by weight of the compressed outer tablet layer;
a disintegrant component comprising from about 5% to about 15% by weight of the compressed outer tablet layer;
optionally, an outer layer wetting agent component comprising from about 0.1% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet;
the outer layer filler/diluent component comprises from about 40% to about 50% by weight of the compressed outer tablet layer;
the outer layer filler/binder component comprises from about 30% to about 40% by weight of the compressed outer tablet layer;
a disintegrant component comprising from about 8% to about 12% by weight of the compressed outer tablet layer;
optionally, an outer layer wetting agent component comprising from about 0.5% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 30% by weight of the core tablet;
the outer layer filler/diluent component comprises from about 40% to about 50% by weight of the compressed outer tablet layer;
the outer layer filler/binder component comprises from about 30% to about 40% by weight of the compressed outer tablet layer;
a disintegrant component comprising from about 8% to about 12% by weight of the compressed outer tablet layer;
optionally, an outer layer wetting agent component comprising from about 0.5% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 25% to about 35% by weight of the core tablet;
the outer layer filler/diluent component comprises from about 40% to about 50% by weight of the compressed outer tablet layer;
the outer layer filler/binder component comprises from about 30% to about 40% by weight of the compressed outer tablet layer;
a disintegrant component comprising from about 8% to about 12% by weight of the compressed outer tablet layer;
optionally, an outer layer wetting agent component comprising from about 0.5% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 5% to about 15% by weight of the core tablet;
the outer layer filler/diluent component comprises from about 40% to about 50% by weight of the compressed outer tablet layer;
the outer layer filler/binder component comprises from about 30% to about 40% by weight of the compressed outer tablet layer;
a disintegrant component comprising from about 9% to about 11% by weight of the compressed outer tablet layer;
optionally, an outer layer wetting agent component comprising from about 1% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 15% to about 30% by weight of the core tablet;
the outer layer filler/diluent component comprises from about 40% to about 50% by weight of the compressed outer tablet layer;
the outer layer filler/binder component comprises from about 30% to about 40% by weight of the compressed outer tablet layer;
a disintegrant component comprising from about 9% to about 11% by weight of the compressed outer tablet layer;
optionally, an outer layer wetting agent component comprising from about 1% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises from about 50% to about 85% by weight of the core tablet;
the core filler/binder component comprises from about 10% to about 20% by weight of the core tablet;
the core hydrophilic gel-forming polymer component comprises from about 25% to about 35% by weight of the core tablet;
the outer layer filler/diluent component comprises from about 40% to about 50% by weight of the compressed outer tablet layer;
the outer layer filler/binder component comprises from about 30% to about 40% by weight of the compressed outer tablet layer;
a disintegrant component comprising from about 9% to about 11% by weight of the compressed outer tablet layer;
optionally, an outer layer wetting agent component comprising from about 1% to about 3% of the compressed outer tablet layer;
optionally, an outer layer lubricant component comprising from about 0.01% to about 2% by weight of the compressed outer tablet layer; and
optionally, an antioxidant component comprising from about 0.01% to about 3% by weight of the compressed outer tablet layer.

In some embodiments of the third aspect of the invention:
the core filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the core filler/binder component comprises one or more of microcrystalline cellulose, polyvinylpyrrolidone, copovidone, polyvinylalcohol, starch, gelatin, gum arabic, gum acacia, and gum tragacanth;
the core hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the outer layer filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the outer layer filler/binder component comprises one or more of microcrystalline cellulose, polyvinylpyrrolidone, copovidone, polyvinylalcohol, starch, gelatin, gum arabic, gum acacia, and gum tragacanth;
the outer layer disintegrant component comprises one or more of croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
the optional outer layer wetting agent component, if present, comprises one or more of a polyethylene glycol-polypropylene glycol copolymer, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, polyethoxylated fatty acid esters, and polyglycolized glycerides;
the optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional antioxidant component, if present, comprises one or more of ascorbic acid, sodium ascorbate, ascorbyl palmitate, vitamin E, vitamin E acetate, butylated hydroxytoluene, and butylated hydroxyanisole;
the core tablet comprises at least one conjugated estrogen; and
the compressed outer tablet layer comprises medroxyprogesterone acetate or bazedoxifene acetate.

In some embodiments of the third aspect of the invention:
the core filler/diluent component comprises one or more of lactose and lactose monohydrate;
the core filler/binder component comprises microcrystalline cellulose;
the core hydrophilic gel-forming polymer component comprises hydroxypropylmethylcellulose;
the optional core lubricant component, if present, comprises magnesium stearate;
the outer layer filler/diluent component comprises one or more of lactose and lactose monohydrate;
the outer layer filler/binder component comprises microcrystalline cellulose;
the outer layer disintegrant component comprises one or more of pregelatinized starch and sodium starch glycolate;
the optional outer layer wetting agent component, if present, comprises a polyethylene glycol-polypropylene glycol copolymer;
the optional outer layer lubricant component, if present, comprises magnesium stearate;
the optional antioxidant component, if present, comprises one or more of ascorbic acid and vitamin E acetate;
the core tablet comprises at least one conjugated estrogen; and
the compressed outer tablet layer comprises medroxyprogesterone acetate or bazedoxifene acetate.

In some embodiments, the present invention provides a tablet-in-tablet composition selected from a plurality of compositions according to the first aspect of the invention, wherein the plurality has a mean dissolution profile wherein:

the mean of % of the estrogen released per composition after 1, 2, 3, 4, and 5 hours under estrogen dissolution conditions is substantially equal to the sum of b₁*X₁, b₂X₂, b₃*X₃, b₁₂*X₁*X₂, b₁₃*X₁*X₃, and b₂₃*X₂*X₃; and

the mean of % of the therapeutic agent per composition released after 0.25, 0.5, 1, 2, and 6 hours under type I therapeutic agent dissolution conditions is substantially equal to the sum of a₁*X₁, b₂X₂, a₃*X₃, a₁₂*X₁*X₂, a₁₃*X₁*X₃, and a₂₃*X₂*X₃;

X₁ is the % by weight of the outer layer hydrophilic gel-forming polymer component in the compressed outer tablet layer;

X₂ is the % by weight of the outer layer filler/diluent component in the compressed outer tablet layer;

X₃ is the % by weight of the outer layer filler/binder component in the compressed outer tablet layer;

b₁ at 1 hour is 157.4;

b₁ at 2 hours is 193.09;

b₁ at 3 hours is 184.1;

b₁ at 4 hours is 146.45;

b₁ at 5 hours is 100.25;

b₂ at 1 hour is 54.47;

b₂ at 2 hours is 80.09;

b₂ at 3 hours is 93.71;

b₂ at 4 hours is 101.05;

b₂ at 5 hours is 104.11;

b₃ at 1 hour is 46.75;

b₃ at 2 hours is 69.86;

b₃ at 3 hours is 84.19;

b₃ at 4 hours is 92.12;

b₃ at 5 hours is 95.89;

b₁₂ at 1 hour is −437.12;

b₁₂ at 2 hours is −557.91;

b₁₂ at 3 hours is −561.48;

b₁₂ at 4 hours is −489.08;

b₁₂ at 5 hours is −383.44;

b₁₃ at 1 hour is −414.17;

b₁₃ at 2 hours is −542.65;

b₁₃ at 3 hours is −569.13;

b₁₃ at 4 hours is −518.63;

b₁₃ at 5 hours is −441.05;

b₂₃ at 1 hour is 76.74;

b₂₃ at 2 hours is 79.7;

b₂₃ at 3 hours is 65.43;

b₂₃ at 4 hours is 43.23;

b₂₃ at 5 hours is 29.91;

a₁ at 0.25 hour is 217.8;

a₁ at 0.5 hour is 218.36;

a₁ at 1 hour is 188.75;

a₁ at 2 hours is 121.23;

a₁ at 6 hours is −21.48;

a₂ at 0.25 hour is 87.91;

a₂ at 0.5 hour is 93.12;

a₂ at 1 hour is 96.98;

a₂ at 2 hours is 100.52;

a₂ at 6 hours is 100.91;

a₃ at 0.25 hour is 58.83;

a₃ at 0.5 hour is 75.08;

a₃ at 1 hour is 86.32;

a₃ at 2 hours is 92.04;

a₃ at 6 hours is 99.99;

a₁₂ at 0.25 hour is −616.98;

a₁₂ at 0.5 hour is −617.39;

a₁₂ at 1 hour is −545.68;

a₁₂ at 2 hours is −377.76;

a₁₂ at 6 hours is 69.72;

a₁₃ at 0.25 hour is −536.63;

a₁₃ at 0.5 hour is −576.95;

a₁₃ at 1 hour is −540.35;

a₁₃ at 2 hours is −397.91;

a₁₃ at 6 hours is 12.22;

a₂₃ at 0.25 hour is 30.77;

a₂₃ at 0.5 hour is 31.94;

a₂₃ at 1 hour is 32.68;

a₂₃ at 2 hours is 32.91; and

a₂₃ at 6 hours is 9.65.

In some embodiments, the present invention provides a tablet-in-tablet composition selected from a plurality of compositions according to the second aspect of the invention, wherein the plurality has a mean dissolution profile wherein:

the mean of % of the estrogen released per composition after 1, 2, 3, 4, and 5 hours under estrogen dissolution conditions is substantially equal to the sum of b₁*X₁, b₂X₂, b₃*X₃, b₁₂*X₁*X₂, b₁₃*X₁*X₃, and b₂₃*X₂*X₃;

the mean of % of the therapeutic agent per composition released after 0.25, 0.5, 1, 2, and 6 hours under type I therapeutic agent dissolution conditions is substantially equal to the sum of a₁*X₁, b₂X₂, a₃*X₃, a₁₂*X₁*X₂, a₁₃*X₁*X₃, and a₂₃*X₂*X₃;

X₁ is the % by weight of the optional outer layer hydrophilic gel-forming polymer component, if present, in the compressed outer tablet layer;

X₂ is the % by weight of the optional outer layer filler/diluent component, if present, in the compressed outer tablet layer; and

X₃ is the % by weight of the optional outer layer filler/binder component, if present, in the compressed outer tablet layer;

b₁ at 1 hour is 157.4;

b₁ at 2 hours is 193.09;

b₁ at 3 hours is 184.1;

b₁ at 4 hours is 146.45;

b₁ at 5 hours is 100.25;

b₂ at 1 hour is 54.47;

b₂ at 2 hours is 80.09;

b₂ at 3 hours is 93.71;

b₂ at 4 hours is 101.05;

b₂ at 5 hours is 104.11;

b₃ at 1 hour is 46.75;

b₃ at 2 hours is 69.86;

b₃ at 3 hours is 84.19;

b₃ at 4 hours is 92.12;

b₃ at 5 hours is 95.89;

b₁₂ at 1 hour is −437.12;

b₁₂ at 2 hours is −557.91;

b₁₂ at 3 hours is −561.48;

b₁₂ at 4 hours is −489.08;

b₁₂ at 5 hours is −383.44;

b₁₃ at 1 hour is −414.17;

b₁₃ at 2 hours is −542.65;

b₁₃ at 3 hours is −569.13;

b₁₃ at 4 hours is −518.63;

b₁₃ at 5 hours is −441.05;

b₂₃ at 1 hour is 76.74;

b₂₃ at 2 hours is 79.7;

b₂₃ at 3 hours is 65.43;

b₂₃ at 4 hours is 43.23;

b₂₃ at 5 hours is 29.91;

a₁ at 0.25 hour is 217.8;

a₁ at 0.5 hour is 218.36;

a₁ at 1 hour is 188.75;

a₁ at 2 hours is 121.23;

a₁ at 6 hours is −21.48;

a₂ at 0.25 hour is 87.91;

a₂ at 0.5 hour is 93.12;

a₂ at 1 hour is 96.98;

a₂ at 2 hours is 100.52;

a₂ at 6 hours is 100.91;

a₃ at 0.25 hour is 58.83;

a₃ at 0.5 hour is 75.08;

a₃ at 1 hour is 86.32;

a₃ at 2 hours is 92.04;

a₃ at 6 hours is 99.99;

a₁₂ at 0.25 hour is −616.98;

a₁₂ at 0.5 hour is −617.39;

a₁₂ at 1 hour is −545.68;

a₁₂ at 2 hours is −377.76;

a₁₂ at 6 hours is 69.72;

a₁₃ at 0.25 hour is −536.63;

a₁₃ at 0.5 hour is −576.95;

a₁₃ at 1 hour is −540.35;

a₁₃ at 2 hours is −397.91;

a₁₃ at 6 hours is 12.22;

a₂₃ at 0.25 hour is 30.77;

a₂₃ at 0.5 hour is 31.94;

a₂₃ at 1 hour is 32.68;

a₂₃ at 2 hours is 32.91; and

a₂₃ at 6 hours is 9.65.

In some embodiments:
the core tablet comprises at least one conjugated estrogen;
the compressed outer tablet layer comprises bazedoxifene acetate;
the dissolution profile of the estrogen from the composition under estrogen dissolution conditions is substantially as shown in any one of FIGS. 30 to 32; and
the dissolution profile of the therapeutic agent from the composition under type II therapeutic agent dissolution conditions is substantially as shown in any one of FIGS. 27 to 29.

In some embodiments:
the core tablet comprises at least one conjugated estrogen;
the compressed outer tablet layer comprises medroxyprogesterone acetate;
the dissolution profile of the estrogen from the composition under estrogen dissolution conditions is substantially as shown in any one of FIGS. 4-6, FIG. 33 (Example 9), FIG. 34 (Example 13), FIG. 35 (Example 15), FIG. 35 (Example 16), FIG. 35 (Example 18) or FIG. 36 (Example 20); and
the dissolution profile of the therapeutic agent from the composition under type I therapeutic agent dissolution conditions is substantially as shown in any one of FIGS. 1-3, FIG. 37 (Example 9), FIG. 38 (Example 13), FIG. 39 (Example 15), FIG. 39 (Example 16), FIG. 39 (Example 18) or FIG. 40 (Example 20).

In some embodiments:
the core tablet comprises at least one conjugated estrogen;
the compressed outer tablet layer comprises medroxyprogesterone acetate;
the dissolution profile of the estrogen from the composition under estrogen dissolution conditions is substantially as shown in any one of FIG. 33 (Example 8), FIG. 33 (Example 10), FIG. 33 (Example 11), FIG. 34 (Example 12), FIG. 34 (Example 14), FIG. 35 (Example 17), FIG. 36 (Example 19) or FIG. 36 (Example 21); and
the dissolution profile of the therapeutic agent from the composition under type I therapeutic agent dissolution conditions is substantially as shown in any one of FIG. 37 (Example 8), FIG. 37 (Example 10), FIG. 38 (Example 11), FIG. 38 (Example 12), FIG. 38 (Example 14), FIG. 39 (Example 17), FIG. 40 (Example 19) or FIG. 40 (Example 21).

In some embodiments, the present invention further provides a tablet-in-tablet composition selected from a plurality of tablet-in-tablet compositions, wherein the plurality has a content uniformity for the therapeutic agent about equal to or less than 2%. In some embodiments, the plurality of tablet-in-tablet compositions has a content uniformity for the therapeutic agent about equal to or less than 1.5%. In some embodiments, the plurality of tablet-in-tablet compositions has a content uniformity for the therapeutic agent about equal to or less than 3.5%. In some embodiments, the plurality of tablet-in-tablet compositions has a content uniformity for the therapeutic agent about equal to or less than 2.5%.

In some embodiments, the present invention further provides a tablet-in-tablet composition selected from a plurality of tablet-in-tablet compositions, wherein the plurality has a weight variation of about equal to or less than 2%. In some embodiments, the plurality of tablet-in-tablet compositions has a weight variation of about equal to or less than 1.5%. In some embodiments, the plurality of tablet-in-tablet compositions has a weight variation of about equal to or less than 3%.

### Processes

The present invention is also directed to processes for producing the tablet-in-tablet compositions of the invention. Accordingly, in one aspect, the present invention provides a process for producing a tablet-in-tablet composition of the invention comprising

compressing a first solid mixture to form a core tablet; and

compressing a second solid mixture onto the core tablet to form a compressed outer tablet layer;

wherein:
(a) the first solid mixture comprises:
one or more estrogens;
a first solid mixture filler/diluent component comprising from about 30% to about 85% by weight of the first solid mixture;
a first solid mixture filler/binder component comprising from about 1% to about 30% by weight of the first solid mixture;
a first solid mixture hydrophilic gel-forming polymer component comprising from about 1% to about 40% by weight of the first solid mixture; and
optionally, a first solid mixture lubricant component comprising from about 0.01% to about 2% by weight of the first solid mixture; and

(b) the second solid mixture comprises:

one or more therapeutic agents selected from the group consisting of selective estrogen receptor modulator and a progestational agent;
a second solid mixture filler/diluent component comprising from about 10% to about 80% by weight of the second solid mixture;
a second solid mixture filler/binder component comprising from about 1% to about 70% by weight of the second solid mixture;
a second solid mixture hydrophilic gel-forming polymer component comprising from about 1% to about 60% of the compressed outer tablet layer;
optionally, a second solid mixture antioxidant component comprising from about 0.01% to about 4% of the second solid mixture; and
optionally, a second solid mixture lubricant component comprising from about 0.01% to about 2% of the second solid mixture.

The first and second solid mixtures can be prepared by a variety of techniques known to one of ordinary skill in the art. In one aspect, the one or both of the first and second solid mixture is prepared by direct blend techniques. In another aspect, one or both of the first and second solid mixture is prepared by wet granulation techniques. In a further aspect, one or both of the first and second solid mixture is prepared by dry granulation processes. Granulation of the mixture can be accomplished by any of the granulation techniques known to one of skill in the art. For example, dry granulation techniques include, but are not limited to, compression of the mixed powder under high pressure, either by roller compaction or “slugging” in a heavy-duty tablet press. Wet granulation techniques include, but are not limited to, high shear granulation, single-pot processing, top-spray granulation, bottom-spray granulation, fluidized spray granulation, extrusion/spheronization, and rotor granulation.

In some embodiments, the process further comprises blending the one or more therapeutic agents, the second solid mixture filler/binder component, the second solid mixture filler/diluent component, and the second solid mixture hydrophilic gel-forming polymer component to form the second solid mixture.

In some embodiments, the blending further comprises:

blending the one or more therapeutic agents and the second solid mixture filler/binder component to form an initial mixture; and

blending the initial mixture with the second solid mixture filler/diluent component and the second solid mixture hydrophilic gel-forming polymer component to form the second solid mixture.

In some embodiments, the process further comprises granulating and then milling the second solid mixture after the blending and prior to the compressing to form the compressed outer tablet layer.

In some embodiments, the process further comprises blending the second solid mixture antioxidant component and, optionally, at least a portion of the optional second solid mixture lubricant component with the one or more therapeutic agents, the second solid mixture filler/binder component, the second solid mixture filler/diluent component, and the second solid mixture hydrophilic gel-forming polymer component to form the second solid mixture.

In some embodiments, the process further comprises blending the first solid mixture filler/diluent component, the first solid mixture filler/binder component, the first solid mixture hydrophilic gel-forming polymer component, and the estrogen to form the first solid mixture.

In some embodiments, the process further comprises granulating and then milling the first solid mixture after the blending.

In some embodiments, the process further comprises the steps of:

(a) adding water to the first solid mixture during the granulating; and

(b) drying the first granulated mixture before the milling.

In some embodiments, the process further comprises drying the first granulated mixture to loss on drying (LOD) of from about 1% to about 3%.

In some embodiments, the process further comprises the steps of:

(i) blending the first solid mixture filler/diluent component, the first solid mixture filler/binder component, the first solid mixture hydrophilic gel-forming polymer component, and the estrogen to form a first solid mixture;

(ii) granulating the first solid mixture of step (i) in the presence of water;

(iiii) drying the first solid mixture of step (ii)

(iv) milling the first solid mixture of step (iii);

(v) optionally, blending the first solid mixture of step (iv) with the optional first solid mixture lubricant component, if present;

(vi) compressing the first solid mixture of step (iv) or step (v), if utilized, to form the core tablet;

(vii) blending the one or more therapeutic agents and the second solid mixture filler/binder component to form an initial mixture;

(viii) blending the initial mixture with the second solid mixture filler/diluent component and the second solid mixture hydrophilic gel-forming polymer component to form a second solid mixture;

(ix) optionally, granulating the second solid mixture of step (viii);

(x) optionally, blending the second solid mixture of step (viii) or step (ix), if utilized, with at least a portion of the optional second solid mixture lubricant component; and

(xi) after step (viii) or steps (ix) or (x), if utilized, compressing the second solid mixture of (vi) onto the core tablet of step (iv) to form the compressed outer tablet layer.

In some embodiments, the first solid mixture filler/diluent component, the first solid mixture filler/binder component, the first solid mixture hydrophilic gel-forming polymer component, or the optional first solid mixture lubricant component are selected from those listed above for the core tablet of the tablet-in-tablet compositions. In some embodiments, the second solid mixture filler/diluent component, the second solid mixture filler/binder component, the second solid mixture hydrophilic gel-forming polymer component, the optional second solid mixture lubricant component, or the optional second solid mixture antioxidant component are selected from those listed above for the compressed outer tablet layer of the tablet-in-tablet compositions.

In some embodiments:
the first solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the first solid mixture filler/binder component comprises one or more of microcrystalline cellulose, polyvinylpyrrolidone, copovidone, polyvinylalcohol, starch, gelatin, gum arabic, gum acacia, and gum tragacanth;
the first solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional first solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the second solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the second solid mixture filler/binder component comprises one or more of microcrystalline cellulose, polyvinylpyrrolidone, copovidone, polyvinylalcohol, starch, gelatin, gum arabic, gum acacia, and gum tragacanth;
the second solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional second solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional second solid mixture antioxidant component, if present, comprises one or more of ascorbic acid, sodium ascorbate, ascorbyl palmitate, vitamin E, vitamin E acetate, butylated hydroxytoluene, and butylated hydroxyanisole;
the core tablet comprises at least one conjugated estrogen; and
the compressed outer tablet layer comprises medroxyprogesterone acetate or bazedoxifene acetate.

In some embodiments:
the first solid mixture filler/diluent component comprises one or more of lactose and lactose monohydrate;
the first solid mixture filler/binder component comprises microcrystalline cellulose;
the first solid mixture hydrophilic gel-forming polymer component comprises hydroxypropylmethylcellulose;
the optional first solid mixture lubricant component, if present, comprises magnesium stearate;
the second solid mixture filler/diluent component comprises one or more of lactose and lactose monohydrate;
the second solid mixture filler/binder component comprises microcrystalline cellulose;
the second solid mixture hydrophilic gel-forming polymer component comprises hydroxypropylmethylcellulose;
the optional second solid mixture lubricant component, if present, comprises magnesium stearate;
the optional second solid mixture antioxidant component, if present, comprises one or more of ascorbic acid and vitamin E acetate;
the core tablet comprises at least one conjugated estrogen; and
the compressed outer tablet layer comprises medroxyprogesterone acetate or bazedoxifene acetate.

In an another aspect, the present invention provides a process for producing a tablet-in-tablet composition comprising:

compressing a first solid mixture to form a core tablet; and

compressing a second solid mixture onto the core tablet to form a compressed outer tablet layer;

wherein:

a) the first solid mixture comprises:

one or more estrogens;

a first solid mixture filler/diluent component comprising from about 30% to about 85% by weight by weight of the core tablet;

a first solid mixture filler/binder component comprising from about 1% to about 30% by weight of the core tablet;

a first solid mixture hydrophilic gel-forming polymer component comprising from about 1% to about 40% by weight of the core tablet; and

optionally, a first solid mixture lubricant component comprising from about 0.01% to about 2% by weight of the core tablet; and

b) the second solid mixture comprises:

one or more therapeutic agents selected from the group consisting of selective estrogen receptor modulators and progestational agents;

a pharmaceutically acceptable carrier component comprising from about 60% to about 99.9% by weight of the compressed outer tablet layer, wherein the outer pharmaceutically acceptable carrier component optionally comprises one or more of a second solid mixture filler/diluent component, a second solid mixture filler/binder component, and a second solid mixture hydrophilic gel-forming polymer component;

optionally, a second solid mixture lubricant component comprising from about 0.01% to about 2% by weight of the compressed outer tablet layer; and

optionally, a second solid mixture antioxidant component comprising from about 0.01% to about 4% by weight of the compressed outer tablet layer.

The first and second solid mixtures can be prepared by various techniques known in the art, including, but not limited to, the techniques described above.

In some embodiments, the process further comprises blending the one or more therapeutic agents and the pharmaceutically acceptable carrier component to form the second solid mixture.

In some embodiments, the process further comprises granulating and then milling the second solid mixture prior to compressing to form the compressed outer tablet layer.

In some embodiments, the process further comprises blending the first solid mixture filler/diluent component, the first solid mixture filler/binder component, the first solid mixture hydrophilic gel-forming polymer component, and the estrogen to form the first solid mixture.

In some embodiments, the process further comprises granulating and then milling the first solid mixture prior to compressing to form the core tablet.

In some embodiments, the process further comprises the steps of:

(a) adding water to the first solid mixture during the granulating; and

(b) drying the first granulated mixture before the milling.

In some embodiments, the process further comprises the steps of:

(i) blending the first solid mixture filler/diluent component, the first solid mixture filler/binder component, the first solid mixture hydrophilic gel-forming polymer component, and the estrogen to form a first solid mixture;

(ii) granulating the first solid mixture of step (i) in the presence of water;

(iii) milling the first solid mixture of step (iii) after the granulating;

(iv) optionally, blending the first solid mixture of step (iii) with the optional first solid mixture lubricant component, if present;

(v) compressing the first solid mixture of step (iiii) or optional step (iv), if utilized, to form the core tablet;

(vi) blending the one or more therapeutic agents and the pharmaceutically acceptable carrier component to form an initial mixture;

(vii) optionally, granulating and then milling the second solid mixture of step (vi);

(viii) optionally, blending the second solid mixture of step (vi) or optional step (vii), if utilized, with at least a portion of the optional second solid mixture lubricant component; and

(ix) after step (vi) or optional steps (vi) and (vii), if utilized, compressing the second solid mixture of (vi) onto the core tablet of step (iv) to form the compressed outer tablet layer.

In some embodiments, the first solid mixture filler/diluent component, the first solid mixture filler/binder component, the first solid mixture hydrophilic gel-forming polymer component, or the optional first solid mixture lubricant component are selected from those listed above for the core tablet of the tablet-in-tablet compositions. In some embodiments, the second solid mixture filler/diluent component, the second solid mixture filler/binder component, the second solid mixture hydrophilic gel-forming polymer component, the optional second solid mixture lubricant component, or the optional second solid mixture antioxidant component are selected from those listed above for the compressed outer tablet layer of the tablet-in-tablet compositions.

In some embodiments:
the first solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the first solid mixture filler/binder component comprises one or more of microcrystalline cellulose, polyvinylpyrrolidone, copovidone, polyvinylalcohol, starch, gelatin, gum arabic, gum acacia, and gum tragacanth;
the first solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the optional first solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the pharmaceutically acceptable carrier component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, a metal carbonate, polyvinylpyrrolidone, copovidone, polyvinylalcohol, gelatin, gum arabic, gum acacia, gum tragacanth, hydroxypropylmethylcellulose, polyethylene oxide, hydroxypropylcellulose, hydroxyethylcellulose, methylcellulose, xanthan gum, and guar gum;
the optional second solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional second solid mixture antioxidant component, if present, comprises one or more of ascorbic acid, sodium ascorbate, ascorbyl palmitate, vitamin E, vitamin E acetate, butylated hydroxytoluene, and butylated hydroxyanisole;
the core tablet comprises at least one conjugated estrogen; and
the compressed outer tablet layer comprises medroxyprogesterone acetate or bazedoxifene acetate.

In some embodiments:
the first solid mixture filler/diluent component comprises one or more of lactose and lactose monohydrate;
the first solid mixture filler/binder component comprises microcrystalline cellulose;
the first solid mixture hydrophilic gel-forming polymer component comprises hydroxypropylmethylcellulose;
the optional first solid mixture lubricant component, if present, comprises magnesium stearate;
the pharmaceutically acceptable carrier component comprises one or more of lactose, lactose monohydrate, microcrystalline cellulose, and hydroxypropylmethylcellulose;
the optional second solid mixture lubricant component, if present, comprises magnesium stearate;
the optional second solid mixture antioxidant component, if present, comprises one or more of ascorbic acid and vitamin E acetate;
the core tablet comprises at least one conjugated estrogen; and
the compressed outer tablet layer comprises medroxyprogesterone acetate or bazedoxifene acetate.

In an another aspect, the present invention provides a process for producing a tablet-in-tablet composition comprising:
compressing a first solid mixture to form a core tablet; and
compressing a second solid mixture onto the core tablet to form a compressed outer tablet layer;
   wherein:

a) the first solid mixture comprises:
one or more estrogens;
a first solid mixture filler/diluent component comprising from about 30% to about 85% by weight of the core tablet;
a first solid mixture filler/binder component comprising from about 1% to about 30% by weight of the core tablet;
a first solid mixture hydrophilic gel-forming polymer component comprising from about 1% to about 40% by weight of the core tablet; and
optionally, a first solid mixture lubricant component comprising from about 0.01% to about 2% by weight of the core tablet; and

b) the second solid mixture comprises:
one or more therapeutic agents selected from the group consisting of selective estrogen receptor modulators and progestational agents;
a second solid mixture filler/diluent component comprising from about 25% to about 65% by weight of the compressed outer tablet layer;
a second solid mixture filler/binder component comprising from about 20% to about 50% by weight of the compressed outer tablet layer;
a second solid mixture disintegrant component comprising from about 2% to about 15% by weight of the compressed outer tablet layer;
optionally, a second solid mixture wetting agent component comprising from about 0.01% to about 4% of the compressed outer tablet layer;
optionally, a second solid mixture lubricant component comprising from about 0.01% to about 2% by weight of the compressed outer tablet layer; and
optionally, a second solid mixture antioxidant component comprising from about 0.01% to about 4% by weight of the compressed outer tablet layer.

The first and second solid mixtures can be prepared by various techniques known in the art, including, but not limited to, the techniques described above.

In some embodiments, the process further comprises blending the first solid mixture filler/diluent component, the first solid mixture filler/binder component, the first solid mixture hydrophilic gel-forming polymer component, and the estrogen to form the first solid mixture.

In some embodiments, the process further comprises granulating and then milling the first solid mixture after the blending.

In some embodiments, the process further comprises the steps of:

(a) adding water to the first solid mixture during the granulating; and

(b) drying the first granulated mixture before the milling.

In some embodiments, the process further comprises drying the first granulated mixture to loss on drying (LOD) of from about 1% to about 3%.

In some embodiments, the process further comprises blending the one or more therapeutic agents, the optional second solid mixture wetting agent component, if present, and the optional second solid mixture antioxidant component, if present, with at least a portion of each of the second solid mixture filler/diluent component, the second solid mixture filler/binder component, and the second solid mixture disintegrant component to form an initial mixture.

In some embodiments, the process further comprises granulating and then milling the initial mixture after the blending to form a granulated mixture.

In some embodiments, the process further comprises blending the granulated mixture with any remaining portion of the second solid mixture filler/diluent component, the second solid mixture filler/binder component and the second solid mixture disintegrant component to form the second solid mixture.

In some embodiments, the process further comprises blending the second solid mixture with the optional second solid mixture lubricant component, if present, prior to compressing the second solid mixture onto the core tablet.

In some embodiments, the process further comprises the steps of:

(i) blending the first solid mixture filler/diluent component, the first solid mixture filler/binder component, the first solid mixture hydrophilic gel-forming polymer component, and the estrogen to form a first solid mixture;

(ii) granulating the first solid mixture of step (i) in the presence of water;

(iiii) drying the first solid mixture of step (ii)

(iv) milling the first solid mixture of step (iii);

(v) optionally, blending the first solid mixture of step (iv) with the optional first solid mixture lubricant component, if present;

(vi) compressing the first solid mixture of step (iv) or step (v), if utilized, to form the core tablet;

(vii) blending the one or more therapeutic agents, the optional second solid mixture wetting agent component, if present, and the optional second solid mixture antioxidant component, if present, with at least a portion of each of the second solid mixture filler/diluent component, the second solid mixture filler/binder component, and the second solid mixture disintegrant component to form an initial mixture;

(viii) optionally, granulating and milling the second solid mixture of step (vii) to form a granulated mixture;

(ix) blending either the initial mixture of (vii) or the granulated mixture of (viii) with any remaining portion of the second solid mixture filler/diluent component, the second solid mixture filler/binder component and the second solid mixture disintegrant component to form the second solid mixture;

(x) optionally, blending the second solid mixture of step (ix) with at least a portion of the optional second solid mixture lubricant component; and

(xi) compressing the second solid mixture of either step (ix) or step (x) onto the core tablet of step (vi) to form the compressed outer tablet layer.

In some embodiments, the first solid mixture filler/diluent component, the first solid mixture filler/binder component, the first solid mixture hydrophilic gel-forming polymer component, or the optional first solid mixture lubricant component are selected from those listed above for the core tablet of the tablet-in-tablet compositions. In some embodiments, the second solid mixture filler/diluent component, the second solid mixture filler/binder component, the second solid mixture disintegrant component, the second solid mixture wetting agent component, the optional second solid mixture lubricant component, or the optional second solid mixture antioxidant component are selected from those listed above for the compressed outer tablet layer of the tablet-in-tablet compositions.

In some embodiments:
the first solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the first solid mixture filler/binder component comprises one or more of microcrystalline cellulose, polyvinylpyrrolidone, copovidone, polyvinylalcohol, starch, gelatin, gum arabic, gum acacia, and gum tragacanth;
the first solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
the first solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the second solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
the second solid mixture filler/binder component comprises one or more of microcrystalline cellulose, polyvinylpyrrolidone, copovidone, polyvinylalcohol, starch, gelatin, gum arabic, gum acacia, and gum tragacanth;
the second solid mixture disintegrant component comprises one or more of croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
the optional second solid mixture wetting agent component, if present, comprises one or more of a polyethylene glycol-polypropylene glycol copolymer, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, polyethoxylated fatty acid esters, and polyglycolized glycerides;
the optional second solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
the optional second solid mixture antioxidant component, if present, comprises one or more of ascorbic acid, sodium ascorbate, ascorbyl palmitate, vitamin E, vitamin E acetate, butylated hydroxytoluene, and butylated hydroxyanisole;
the core tablet comprises at least one conjugated estrogen; and
the compressed outer tablet layer comprises medroxyprogesterone acetate or bazedoxifene acetate.

In some embodiments:
the first solid mixture filler/diluent component comprises one or more of lactose and lactose monohydrate;
the first solid mixture filler/binder component comprises microcrystalline cellulose;
the first solid mixture hydrophilic gel-forming polymer component comprises hydroxypropylmethylcellulose;
the optional first solid mixture lubricant component, if present, comprises magnesium stearate;
the second solid mixture filler/diluent component comprises one or more of lactose and lactose monohydrate;
the second solid mixture filler/binder component comprises microcrystalline cellulose;
the second solid mixture disintegrant component comprises one or more of pregelatinized starch and sodium starch glycolate;
the optional second solid mixture wetting agent component, if present, comprises a polyethylene glycol-polypropylene glycol copolymer;
the optional second solid mixture lubricant component, if present, comprises magnesium stearate;
the optional second solid mixture component, if present, comprises one or more of ascorbic acid and vitamin E acetate;
the core tablet comprises at least one conjugated estrogen; and
the compressed outer tablet layer comprises medroxyprogesterone acetate or bazedoxifene acetate.

In some embodiments, the processes produce a plurality of tablet-in-tablet compositions having a content uniformity for the therapeutic agent about equal to or less than 3.5%. In some embodiments, the processes produce a plurality of tablet-in-tablet compositions having a content uniformity for the therapeutic agent about equal to or less than 2.5%. In some embodiments, the processes produce a plurality of tablet-in-tablet compositions having a content uniformity for the therapeutic agent about equal to or less than 2% or 1.5%.

In some embodiments, the processes produce a plurality of tablet-in-tablet compositions having a weight variation about equal to or less than 2%. In some embodiments, the processes produce a plurality of tablet-in-tablet compositions having a weight variation about equal to or less than 1.5%.

The processes described herein can be used to prepare any of the tablet-in-tablet compositions described herein, or combinations or subcombinations thereof.

The present invention further provides products produced by the processes of the invention. Any of the embodiments of the processes described herein, or subembodiments or subcombinations thereof, can be used to produce the products of the invention.

In some embodiments, the compressed outer tablet layer of the product has a hardness from about 2 kp to about 7 kp.

In general, the estrogen and therapeutic agent in the compositions and mixtures described herein are present in a pharmaceutically effective amount. The phrase “pharmaceutically effective amount” refers to the amount of the active pharmacological agent that elicits the biological or medicinal response in a tissue, system, animal, individual, patient, or human that is being sought by a researcher, veterinarian, medical doctor or other clinician. The desired biological or medicinal response may include preventing the disorder in a patient (e.g., preventing the disorder in a patient that may be predisposed to the disorder, but does not yet experience or display the pathology or symptomatology of the disease). The desired biological or medicinal response may also include inhibiting the disorder in a patient that is experiencing or displaying the pathology or symptomatology of the disorder (i.e., arresting or slowing further development of the pathology and/or symptomatology). The desired biological or medicinal response may also include ameliorating the disorder in a patient that is experiencing or displaying the pathology or symptomatology of the disease (i.e., reversing the pathology or symptomatology).

The pharmaceutically effective amount provided in the prophylaxis or treatment of a specific disorder may vary according to the specific condition(s) being treated, the size, age and response pattern of the patient, the severity of the disorder, the judgment of the attending physician or the like. In general, effective amounts for daily oral administration may be about 0.01 to 1,000 mg/kg, or about 0.5 to 500 mg/kg.

In general, the compositions can be administered by any appropriate route, for example, orally. The excipients of the compositions and mixtures can also be combined with mixtures of other active compounds or inert fillers and/or diluents. Additional numerous various excipients, dosage forms, dispersing agents and the like that are suitable for use in connection with the compositions of the invention are known in the art and described in, for example, Remington's Pharmaceutical Sciences, 17th ed., Mack Publishing Company, Easton, Pa., 1985, which is incorporated herein by reference in its entirety.

Film coatings useful with the present compositions are known in the art and generally consist of a polymer (usually a cellulosic type of polymer), a colorant and a plasticizer. The compositions and formulations herein may also be combined and processed as a solid, then placed in a capsule form such as a gelatin capsule. In some cases, a plasticizer may be formulated into the outer tablet layer to prevent cracking.

Certain features of the invention are described herein in embodiments. It is emphasized that certain features of the invention, which are, for clarity, described herein in the context of separate embodiments, can also be provided in combination in a single embodiment, unless otherwise specified. Conversely, various features of the invention which are, for brevity, described in the context of a single embodiment, can also be provided separately or in any suitable subcombination, unless otherwise specified. For example, some of the embodiments herein describe individual weight percentages for each excipient, estrogen, or therapeutic agent in a given portion of the composition or mixture, while other embodiments herein describe the chemical composition of the excipients, estrogens, or therapeutic agents; these embodiments can also be provided in any suitable combination or subcombination, as well as being provided separately in a single embodiment, unless otherwise specified.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of Conjugated Estrogens Granule Comprising 27.5% HPMC K100M and Compression of the Granule to a Tablet Form

One feature of the tablet form described herein is a core tablet comprising, e.g., conjugated estrogens. Examples 1-3 are examples demonstrating production of a conjugated estrogen (“CE”) granule. In this Example 1, to produce a CE granule, HPMC K100M Premium Controlled Release (CR) grade (Dow Chemical Co., Midland, Mich.) was selected for use based on its controlled release properties. HPMC

Premium CR grade is specially produced ultra-fine particle size material, which can ensure a rapid hydration and gel formation.

CE Desiccation with Lactose (“CEDL”) (Wyeth, Madison, N.J.) was used. CEDL at a 42.9 mg CE/g mixture was granulated with the balance of the remaining ingredients in Table 1 (with the incorporation) of water by means of a high shear granulator following the procedures below for a batch size of 1.5 kg by following the procedure below.

1. CEDL was mixed with Lactose Monohydrate Spray Dried (Wyeth, Madison, N.J.), AVICEL® PH 101 (FMC Biopolymer, Philadelphia, Pa.) and HPMC K100M Premium CR (Dow Chemical Co., Midland, Mich.) in a 10 liter Collette high shear mixer for 5 minutes with plows at approximately 430 rpm.
2. The blend of step 1 was granulated by initiating the addition of water to the Collette mixer with plows and choppers running at approximately 430 and 1800 rpm, respectively. All of the water was added within approximately 4 minutes.
3. The granulation was continued for approximately 7 minutes.
4. The wet granulation was dried in a fluid bed dryer at an inlet temperature set-point of 60° C. to achieve a target granulation loss on drying (“LOD”) of 2%. A variation of ±0.5% moisture content was acceptable.
5. The dried granulation was passed through a Model “M” Fitzmill equipped with a #2A plate, set at a high speed (4500-4600 rpm), and impact knives set forward.
6. The granulation of step 5 was mixed in a V-Blender for approximately 10 minutes at approximately 22 rpm.
7. About 100 g of the blend of step 6 was removed for use in step 8.
8. Magnesium stearate (“MS”) was added through a #20 screen, in approximately equal portions, to each side of the V-blender. After the MS addition, the blend of step 7 was added, in approximately equal portions, to each side of the V-blender and blended for approximately 3 minutes. The quantity of MS added was adjusted on a per tablet basis based on the quantity of granulation to be blended.
9. The step 8 lubricated granulation was discharged into a double-bagged polyethylene bag with a desiccant bag between the bags.
10. The lubricated CE granulation was then compressed into 120 mg tablets using a ¼ inch round convex tooling with a Korsch XL100 compression machine. The tablets have a hardness range of 7.5-9.5 kp and thickness range of 0.14-0.16 inches.

TABLE 1

| Description | Input/Tablet (mg) | % W/W |
|---|---|---|
| CE Desiccation with Lactose at 42.9 mg/g | 10.4895 | 8.74 |
| Lactose Monohydrate Spray Dried | 58.2105 | 48.51 |
| Avicel PH 101, NF | 18 | 15.00 |
| HPMC K100M Premium CR | 33 | 27.50 |
| Magnesium Stearate, NF | 0.3 | 0.25 |
| Purified Water, USP (A) | 30 | |

Note:
(A) Indicates removed during processing.

### Example 2

### Preparation of Conjugated Estrogens Granule Comprising 20% HPMC K100M and Compression of the Granule to a Tablet Form

Using the ingredient amounts in Table 2, a granulated CE mixture was prepared and used to form a tablet by following the procedure of Example 1.

TABLE 2

| Description | Input/Tablet (mg) | % W/W |
|---|---|---|
| CE Desiccation with Lactose at 42.9 mg/g | 10.4895 | 8.74 |
| Lactose Monohydrate Spray Dried | 67.2105 | 56.01 |
| Avicel PH 101, NF | 18 | 15.00 |
| HPMC K100M Premium CR | 24 | 20.00 |
| Magnesium Stearate, NF | 0.3 | 0.25 |
| Purified Water, USP (A) | 30 | |

Note:
(A) Indicates removed during processing.

### Example 3

### Preparation of Conjugated Estrogens Granule Comprising 10% HPMC K100M and Compression of the Granule to a Tablet Form

Using the ingredient amounts in Table 3, a granulated CE mixture was prepared and used to form a tablet by following the procedure of Example 1.

TABLE 3

| Description | Input/Tablet (mg) | % W/W |
|---|---|---|
| CE Desiccation with Lactose at 42.9 mg/g | 10.4895 | 8.74 |
| Lactose Monohydrate Spray Dried | 79.2105 | 66.01 |
| Avicel PH 101, NF | 18 | 15.00 |
| HPMC K100M Premium CR | 12 | 10.00 |
| Magnesium Stearate, NF | 0.3 | 0.25 |
| Purified Water, USP (A) | 30 | |

Note:
(A) Indicates removed during processing.

### Examples 4-21

Another feature of the tablet described herein is an outer layer comprising a selected drug such as a progesterone. Examples 4-21 detail the preparation of blends of medroxyprogesterone acetate (“MPA”) with varying amounts of Lactose Monohydrate Spray Dried (Foremost Farms USA, Baraboo, Wis.), AVICEL® PH 200 (FMC Biopolymer, Philadelphia, Pa.), and HPMC K100M Premium CR (Dow Chemical Co., Midland, Mich.) for use as an outer layer. Lactose Monohydrate Spray Dried, AVICEL® PH 200, and HPMC K100M Premium CR are excipients in these blends. Some blends do not contain one or more of these excipients.

### Example 4

### Preparation of Medroxyprogesterone Acetate Blend Comprising 20% HPMC K4M

Using the ingredient amounts in Table 4, a blend of MPA was formed by the following procedure.

1. MPA (Berlichem, Inc., Fairfield, N.J.) was screened together with AVICEL® PH 200 (FMC Biopolymer, Philadelphia, Pa.) through a #20 mesh screen.
2. The step 1 mixture was blended in a V-blender for approximately 110 revolutions.
3. The lactose and HPMC were screened through the same screen and added to the blender.
4. The step 3 mixture was blended for approximately 330 revolutions.
5. Magnesium stearate (“MS”) was screened together with approximately 100 g of the blend of step 4 through the same screen and add to the blender. This mixture was then blended for approximately 66 revolutions and then discharged.

TABLE 4

| Description | Input/Tablet (mg) | % W/W |
|---|---|---|
| Medroxyprogesterone Acetate, USP, | 1.5 | 0.63 |
| Micronized @ 100% | | |
| Lactose Monohydrate Spray Dried, NF | 93.9 | 39.13 |
| Microcrystalline Cellulose, NF | 96 | 40.00 |
| (Avicel PH 200) | | |
| HPMC K4M Premium CR | 48 | 20.00 |
| Magnesium Stearate, NF | 0.6 | 0.25 |

### Example 5

### Preparation of Tablet-In-Tablet Composition

To prepare a tablet-in-tablet composition, the MPA blend of Example 4 was compressed onto the CE internal tablet of Example 1 with an 11 mm round convex tooling utilizing a Kilian RUD compression machine. The target MPA external layer weight was 240 mg, which generated a target tablet-in-tablet weight of 360 mg. The fill weights of both sides (top and bottom) were adjusted to allow the CE internal tablet to position itself at the center of the finished tablet. Since the hardness measurement of tablet-in-tablet was not consistent due to capping during the testing, which is a common problem for tablet-in-tablet compositions, the compression force was based on the hardness of the tablet with the MPA external layer only. The targeted hardness of the MPA outer layer tablet alone had a range of 2.0-6.0 kp. Under this compression force, the tablet-in-tablet composition had a friability of zero percent.

### Example 6

### Preparation of Tablet-In-Tablet Composition

Using the MPA blend of Example 4 and the CE internal tablet of Example 2, a tablet-in-tablet composition was prepared by following the procedure of Example 5.

### Example 7

### Preparation of Tablet-In-Tablet Composition

Using the MPA blend of Example 4 and the CE internal tablet of Example 3, a tablet-in-tablet composition was prepared by following the procedure of Example 5.

### Example 8

### Preparation of Medroxyprogesterone Acetate Blend and Subsequent Formation of a Tablet-In-Tablet Composition

Using the ingredient amounts in Table 5, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen and blended together in a 4 Qt V-blender for approximately 110 revolutions.
2. The lactose was added into the blender and blended for approximately 330 revolutions.
3. MS was screened with about 100 g of blended material through the #30 mesh screen.
4. The mixture of step 3 was added into the blender and blended for about 66 revolutions.

This MPA mixture of step 4 was then compressed onto the CE internal tablet of Example 3 to form a tablet-in-tablet composition using the procedure in Example 5.

TABLE 5

| Ingredients | mg/tab | w/w % | g/batch |
|---|---|---|---|
| MPA Micronized | 1.5 | 0.63 | 6.25 |
| Lactose monohydrate spray dried | 112.95 | 47.07 | 470.63 |
| Avicel PH 200 | 124.95 | 52.07 | 520.63 |
| HPMC K4M Premium CR | 0 | 0.00 | 0.00 |
| Magnesium Stearate | 0.6 | 0.25 | 2.50 |
| Total | 240.00 | 100.0 | 1000.00 |

### Example 9

### Preparation of Medroxyprogesterone Acetate Blend and Subsequent Formation of a Tablet-In-Tablet Composition

Using the ingredient amounts in Table 6, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1, lactose monohydrate spray dried (Foremost Farms USA, Baraboo, Wis.), and HPMC were added to a 2 Qt V-blender and blended for approximately 440 revolutions.
3. MS was screened with about 100 g of blended material through the #30 mesh screen.
4. The mixture of step 3 was added to the blender and blended for about 66 revolutions.

This MPA mixture of step 4 was then compressed onto the CE internal tablet of Example 3 to form a tablet-in-tablet composition using the procedure in Example 5.

TABLE 6

| Ingredients | mg/tab | w/w % | g/batch |
|---|---|---|---|
| MPA Micronized | 1.5 | 0.63 | 3.13 |
| Lactose monohydrate spray | 165.900 | 69.13 | 345.65 |
| dried | | | |
| Avicel PH 200 | 12 | 5.00 | 25.00 |
| HPMC K4M Premium CR | 60 | 25.00 | 125.00 |
| Magnesium Stearate | 0.6 | 0.25 | 1.25 |
| Total | 240.00 | 100.0 | 500.03 |

### Example 10

### Preparation of Medroxyprogesterone Acetate Blend and Subsequent Formation of a Tablet-In-Tablet Composition

Using the ingredient amounts in Table 7, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen and blended together in a 4 Qt V-blender for approximately 440 revolutions.
2. MS was screened with about 100 g of blended material through the #30 mesh screen.
3. The mixture of step 2 was added to the blender and blended for about 66 revolutions.

The MPA mixture of step 4 was then compressed onto the CE internal tablet of Example 3 to form a tablet-in-tablet composition using the procedure in Example 5.

TABLE 7

| Ingredients | mg/tab | w/w % | g/batch |
|---|---|---|---|
| MPA Micronized | 1.5 | 0.63 | 6.25 |
| Lactose monohydrate spray dried | 0 | 0.00 | 0.00 |
| Avicel PH 200 | 237.9 | 99.13 | 991.25 |
| HPMC K4M Premium CR | 0 | 0.00 | 0.00 |
| Magnesium Stearate | 0.6 | 0.25 | 2.50 |
| Total | 240.00 | 100.0 | 1000.00 |

### Example 11

### Preparation of Medroxyprogesterone Acetate Blend and Subsequent Formation of a Tablet-In-Tablet Composition

Using the ingredient amounts in Table 8, a MPA blend was formed by the following procedure.

1. AVICEL® PH 200 (FMC Biopolymer, Philadelphia, Pa.) and MPA (Berlichem, Inc., Fairfield, N.J.), and lactose were passed through a #30 mesh screen.
2. The mixture of step 1 was added to a 4 Qt V-blender and blended for approximately 440 revolutions.
3. MS was screened with about 100 g of blended material through the #30 mesh screen.
4. The mixture of step 3 was added to the blender and blended for about 66 revolutions.

The MPA mixture of step 4 was then compressed onto the CE internal tablet of Example 3 to form a tablet-in-tablet composition using the procedure in Example 5.

TABLE 8

| Ingredients | mg/tab | w/w % | g/batch |
|---|---|---|---|
| MPA Micronized | 1.5 | 0.63 | 6.25 |
| Lactose monohydrate spray dried | 225.9 | 94.13 | 941.25 |
| Avicel PH 200 | 12 | 5.00 | 50.00 |
| HPMC K4M Premium CR | 0 | 0.00 | 0.00 |
| Magnesium Stearate | 0.6 | 0.25 | 2.50 |
| Total | 240.00 | 100.0 | 1000.00 |

### Example 12

### Preparation of Medroxyprogesterone Acetate Blend and Subsequent Formation of a Tablet-In-Tablet Composition

Using the ingredient amounts in Table 9, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1 was added to a 4 Qt V-blender and blended for approximately 110 revolutions.
3. Lactose monohydrate spray dried (Foremost Farms USA, Baraboo, Wis.) was added and the mixture was blended for approximately 330 revolutions.
4. MS was screened with about 100 g of blended material through the #30 mesh screen.
5. The mixture of step 4 was added to the blender and blended for about 66 revolutions.

The MPA mixture of step 5 was then compressed onto the CE internal tablet of Example 3 to form a tablet-in-tablet composition using the procedure in Example 5.

TABLE 9

| Ingredients | mg/tab | w/w % | g/batch |
|---|---|---|---|
| MPA Micronized | 1.5 | 0.63 | 6.25 |
| Lactose monohydrate spray dried | 112.95 | 47.07 | 470.63 |
| Avicel PH 200 | 124.95 | 52.07 | 520.63 |
| HPMC K4M Premium CR | 0 | 0.00 | 0.00 |
| Magnesium Stearate | 0.6 | 0.25 | 2.50 |
| Total | 240.00 | 100.0 | 1000.00 |

### Example 13

### Preparation of Medroxyprogesterone Acetate Blend and Subsequent Formation of a Tablet-In-Tablet Composition

Using the ingredient amounts in Table 10, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1, lactose monohydrate spray dried (Foremost Farms USA, Baraboo, Wis.), and HPMC was added into a 2 Qt V-blender and blended for approximately 440 revolutions.
3. MS was screened with about 100 g of blended material through the #30 mesh screen.
4. The mixture of step 3 was added to the blender and blended for about 66 revolutions.

The MPA mixture of step 4 was then compressed onto the CE internal tablet of Example 3 to form a tablet-in-tablet composition using the procedure in Example 5.

TABLE 10

| Ingredients | mg/tab | w/w % | g/batch |
|---|---|---|---|
| MPA Micronized | 1.5 | 0.63 | 3.13 |
| Lactose monohydrate spray dried | 105.9 | 44.13 | 220.65 |
| Avicel PH 200 | 12 | 5.00 | 25.00 |
| HPMC K4M Premium CR | 120 | 50.00 | 250.00 |
| Magnesium Stearate | 0.6 | 0.25 | 1.25 |
| Total | 240.00 | 100.0 | 500.03 |

### Example 14

### Preparation of Medroxyprogesterone Acetate Blend and Subsequent Formation of a Tablet-In-Tablet Composition

Using the ingredient amounts in Table 11, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1 was added to a 2-Qt V-Blender and blended for approximately 110 revolutions.
3. HPMC was added to the blender and blended for approximately 330 revolutions.
4. MS was screened with about 100 g of blended material through the #30 mesh screen.
5. The mixture of step 4 was added into the blender and blended for about 66 revolutions.

The MPA mixture of step 5 was then compressed onto the CE internal tablet of Example 3 to form a tablet-in-tablet composition using the procedure in Example 5.

TABLE 11

| Ingredients | mg/tab | w/w % | g/batch |
|---|---|---|---|
| MPA Micronized | 1.5 | 0.63 | 3.13 |
| Lactose monohydrate spray dried | 0 | 0.00 | 0.00 |
| Avicel PH 200 | 117.9 | 49.13 | 245.65 |
| HPMC K4M Premium CR | 120 | 50.00 | 250.00 |
| Magnesium Stearate | 0.6 | 0.25 | 1.25 |
| Total | 240.00 | 100.0 | 500.03 |

### Example 15

### Preparation of Medroxyprogesterone Acetate Blend and Subsequent Formation of a Tablet-In-Tablet Composition

Using the ingredient amounts in Table 12, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1, lactose and HPMC was added into a 2 Qt V-blender and blended for approximately 440 revolutions.
3. MS was screened with about 100 g of blended material through the #30 mesh screen.
4. The mixture of step 3 was added into the blender and blended for about 66 revolutions.

The MPA mixture of step 4 was then compressed onto the CE internal tablet of Example 3 to form a tablet-in-tablet composition using the procedure in Example 5.

TABLE 12

| Ingredients | mg/tab | w/w % | g/batch |
|---|---|---|---|
| MPA Micronized | 1.5 | 0.63 | 3.13 |
| Lactose monohydrate spray dried | 105.9 | 44.13 | 220.65 |
| Avicel PH 200 | 12 | 5.00 | 25.00 |
| HPMC K4M Premium CR | 120 | 50.00 | 250.00 |
| Magnesium Stearate | 0.6 | 0.25 | 1.25 |
| Total | 240.00 | 100.0 | 500.03 |

### Example 16

### Preparation of Medroxyprogesterone Acetate Blend and Subsequent Formation of a Tablet-In-Tablet Composition

Using the ingredient amounts in Table 13, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1 was added into a 2 Qt V-blender and blended for approximately 110 revolutions.
3. HPMC and lactose was added to the blender and blended for approximately 330 revolutions.
4. MS was screened with about 100 g of blended material through the #30 mesh screen.
5. The mixture of step 4 was added into the blender and blended for about 66 revolutions.

The MPA mixture of step 5 was then compressed onto the CE internal tablet of Example 3 to form a tablet-in-tablet composition using the procedure in Example 5.

TABLE 13

| Ingredients | mg/tab | w/w % | g/batch |
|---|---|---|---|
| MPA Micronized | 1.5 | 0.63 | 3.13 |
| Lactose monohydrate spray dried | 154.428 | 64.35 | 321.75 |
| Avicel PH 200 | 53.472 | 22.28 | 111.40 |
| HPMC K4M Premium CR | 30 | 12.50 | 62.50 |
| Magnesium Stearate | 0.6 | 0.25 | 1.25 |
| Total | 240.00 | 100.0 | 500.03 |

### Example 17

### Preparation of Medroxyprogesterone Acetate Blend and Subsequent Formation of a Tablet-In-Tablet Composition

Using the ingredient amounts in Table 14, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen and blended together in a 4 Qt V-blender for approximately 440 revolutions.
2. MS was screened with about 100 g of blended material through the #30 mesh screen.
3. The mixture of step 2 was added into the blender and blended for about 66 revolutions.

The MPA mixture of step 3 was then compressed onto the CE internal tablet of Example 3 to form a tablet-in-tablet composition using the procedure in Example 5.

TABLE 14

| Ingredients | mg/tab | w/w % | g/batch |
|---|---|---|---|
| MPA Micronized | 1.5 | 0.63 | 6.25 |
| Lactose monohydrate spray dried | 0 | 0.00 | 0.00 |
| Avicel PH 200 | 237.9 | 99.13 | 991.25 |
| HPMC K4M Premium CR | 0 | 0.00 | 0.00 |
| Magnesium Stearate | 0.6 | 0.25 | 2.50 |
| Total | 240.00 | 100.0 | 1000.00 |

### Example 18

### Preparation of Medroxyprogesterone Acetate Blend and Subsequent Formation of a Tablet-In-Tablet Composition

Using the ingredient amounts in Table 15, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1 was added into a 2 Qt V-blender and blended for approximately 110 revolutions.
3. HPMC and lactose monohydrate spray dried (Foremost Farms USA, Baraboo, Wis.) was added to the blender and blended for approximately 330 revolutions.
4. MS was screened with about 100 g of blended material through the #30 mesh screen.
5. The mixture of step 4 was added into the blender and blended for about 66 revolutions.

The MPA mixture of step 5 was then compressed onto the CE internal tablet of Example 3 to form a tablet-in-tablet composition using the procedure in Example 5.

TABLE 15

| Ingredients | mg/tab | w/w % | g/batch |
|---|---|---|---|
| MPA Micronized | 1.5 | 0.63 | 3.13 |
| Lactose monohydrate spray dried | 41.46 | 17.28 | 86.40 |
| Avicel PH 200 | 136.44 | 56.85 | 284.25 |
| HPMC K4M Premium CR | 60 | 25.00 | 125.00 |
| Magnesium Stearate | 0.6 | 0.25 | 1.25 |
| Total | 240.00 | 100.0 | 500.03 |

### Example 19

### Preparation of Medroxyprogesterone Acetate Blend and Subsequent Formation of a Tablet-In-Tablet Composition

Using the ingredient amounts in Table 16, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1 was added into a 2 Qt V-blender and blended for approximately 110 revolutions.
3. HPMC was added to the blender and blended for approximately 330 revolutions.
4. MS was screened with about 100 g of blended material through the #30 mesh screen.
5. The mixture of step 4 was added into the blender and blended for about 66 revolutions.

The MPA mixture of step 5 was then compressed onto the CE internal tablet of Example 3 to form a tablet-in-tablet composition using the procedure in Example 5.

TABLE 16

| Ingredients | mg/tab | w/w % | g/batch |
|---|---|---|---|
| MPA Micronized | 1.5 | 0.63 | 3.13 |
| Lactose monohydrate spray dried | 0 | 0.00 | 0.00 |
| Avicel PH 200 | 177.9 | 74.13 | 370.65 |
| HPMC K4M Premium CR | 60 | 25.00 | 125.00 |
| Magnesium Stearate | 0.6 | 0.25 | 1.25 |
| Total | 240.00 | 100.0 | 500.03 |

### Example 20

### Preparation of Medroxyprogesterone Acetate Blend and Subsequent Formation of a Tablet-In-Tablet Composition

Using the ingredient amounts in Table 17, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen.
2. The mixture of step 1 was added into a 2 Qt V-blender and blended for approximately 110 revolutions.
3. HPMC and lactose monohydrate spray dried (Foremost Farms USA, Baraboo, Wis.) was added to the blender and blended for approximately 330 revolutions.
4. MS was screened with about 100 g of blended material through the #30 mesh screen.
5. The mixture of step 4 was added into the blender and blended for about 66 revolutions.

The MPA mixture of step 5 was then compressed onto the CE internal tablet of Example 3 to form a tablet-in-tablet composition using the procedure in Example 5.

TABLE 17

| Ingredients | mg/tab | w/w % | g/batch |
|---|---|---|---|
| MPA Micronized | 1.5 | 0.63 | 3.13 |
| Lactose monohydrate spray dried | 67.95 | 28.32 | 141.58 |
| Avicel PH 200 | 79.95 | 33.32 | 166.58 |
| HPMC K4M Premium CR | 90 | 37.50 | 187.50 |
| Magnesium Stearate | 0.6 | 0.25 | 1.25 |
| Total | 240.00 | 100.0 | 500.03 |

### Example 21

### Preparation of Medroxyprogesterone Acetate Blend and Subsequent Formation of a Tablet-In-Tablet Composition

Using the ingredient amounts in Table 18, an MPA blend was formed by the following procedure.

1. AVICEL® PH 200 (FMC Biopolymer, Philadelphia, Pa.) and MPA (Berlichem, Inc., Fairfield, N.J.) were passed through a #30 mesh screen and blended together in a 4 Qt V-blender for approximately 440 revolutions.
2. The mixture of step 1 and lactose monohydrate spray dried (Foremost Farms USA, Baraboo, Wis.) was added to a 4 Qt V-blender and blended for approximately 440 revolutions.
3. MS was screened with about 100 g of blended material through the #30 mesh screen.
4. The mixture of step 3 was added into the blender and blended for about 66 revolutions.

The MPA mixture of step 4 was then compressed onto the CE internal tablet of Example 3 to form a tablet-in-tablet composition using the procedure in Example 5.

TABLE 18

| | | | |
|---|---|---|---|
| MPA Micronized | 1.5 | 0.63 | 6.25 |
| Lactose monohydrate spray dried | 225.9 | 94.13 | 941.25 |
| Avicel PH 200 | 12 | 5.00 | 50.00 |
| HPMC K4M Premium CR | 0 | 0.00 | 0.00 |
| Magnesium Stearate | 0.6 | 0.25 | 2.50 |
| Total | 240.00 | 100.0 | 1000.00 |

### Example 22

### Characterization of the CE/MPA Tablet-In-Tablet Compositions

### Weight Variation

Weight variation of 100 tablets was evaluated. The weight of each individual tablet was measured using the Mocon Automatic Balance Analysis tester (USP Method <905>, General Chapters, Uniformity of Dosage Forms). The mean, standard deviation, and relative standard deviation of these 100 values was calculated by the tester. The weight variation is represented by the relative standard deviation. The results are shown in Table 19.

### Content Uniformity of MPA and CE

Content uniformity of MPA and CE was determined on a sample of 10 tablets according to USP Method <905>. The results are shown in Table 19.

Dissolution of MPA from Tablet-in-tablet

The dissolution of MPA from the tablet-in-tablet compositions was determined using USP Apparatus 2, at 50 rpm in 900 mL with 0.54% sodium lauryl sulfate (SLS) in water for a period of 12 hours. Filtered samples of the dissolution medium were taken at specified time intervals. The release of the active was determined by reversed phase high performance liquid chromatography (HPLC). The results are shown in Tables 20 and 22.

Dissolution of Conjugated Estrogens from Tablet-in-tablet

The dissolution of CE from the tablet-in-tablet compositions of Examples 5, 6 and 7 was determined using USP Apparatus 2, at 50 rpm in 900 mL of 0.02 M sodium acetate buffer, pH 4.5, for a period of 8 hours. Filtered samples of the dissolution medium were taken at specified time intervals. The release of the active was determined on the HPLC reversed phase chromatography. The results are shown in Tables 21 and 23.

### Olfactory Screening of Tablet-in-tablets

The CE/MPA tablet-in-tablets were coated with approximately 3% Opadry White (Colorcon, West Point, Pa.). Forty coated/wax polished and uncoated tablets were packaged into 40 ml high density polyethylene (“HDPE”) bottles, respectively. These capped bottles, which were not induction sealed, were put into stability chambers under 40° C./75% RH and 25° C./60% RH conditions, respectively. The bottles were opened weekly. Two different individuals monitored for the characteristic odor of conjugated estrogens.

### Results

The effect of the level of HPMC K100M in the CE internal tablet portion on dissolution rate related to CE and MPA was investigated. All three tested formulations had the same MPA external layer composition. However, the level of HPMC K100M CR in the CE internal tablet portion differed. The compressed tablet-in-tablet product was tested and evaluated for weight variation, content uniformity and dissolution for both CE and MPA active components. The data in Table 19 indicate that the formulation, as well as the process, produced tablet-in-tablet compositions that yield excellent weight variation as well as good content uniformity for both CE and MPA active components. From the results shown in Tables 20 and 21, it can be concluded that the higher the content of the polymer in the CE internal tablet portion, the slower the dissolution rate of CE. On the other hand, the dissolution rate of MPA is not affected by the concentration of HPMC K100M CR in the CE internal tablet portion. Therefore, the effect of an excipient up CE dissolution rate is generally unpredictable.

A D-optimal mixture experimental design was used to optimize the MPA external tablet portion formulation and evaluate the influence of each ingredient on the dissolution rates of MPA and CE. The results from these experiments were analyzed using DESIGN EXPERT® 6.09 software. Table 23 and FIGS. 33-36 display the CE dissolution results from the fourteen formulations generated from experimental design batches. Table 22 and FIGS. 37-40 display the MPA dissolution results from the fourteen formulations generated from experimental design batches. The CE released percentages at 1, 2, 3, 4 and 5 hours, and the MPA released percentages at 15, 30, 60, 120, and 360 minutes of all model formulations were treated by DESIGN EXPERT® 6.09 software. Suitable models for these experiments include linear, quadratic and special cubic models. The best fitting mathematical model was selected based on the comparisons of several statistical parameters including the standard deviation (ST), the multiple correlation coefficient (R²), adjusted multiple correlation coefficient (adjusted R²), predicted multiple correlation coefficient (predicted R²), the predicted residual sum of square (PRESS), and adequate precision provided by DESIGN EXPERT® 6.09 software. Among these statistical parameters, PRESS indicates how well the model fits the data, and for the chosen model it should be small relative to the other models under consideration. The predicted R² has been in reasonable agreement with the adjusted R². The adequate precision measures the signal to noise ratio. A ratio greater than 4 is desirable.

### Linear Model:

Y=b₁X₁+b₂X₂+b₃X₃

### Quadratic Model:

Y=b₁X₁+b₂X₂+b₃X₃+b₁₂X₁X₂+b₁₃X₁X₃+b₂₃X₂X₃

### Special Cubic Model:

Y=b₁X₁+b₂X₂+b₃X₃+b₁₂X₁X₂+b₁₃X₁X₃+b₂₃X₂X₃+b₁₂₃X₁X₂X₃

### Note:

X₁: Level of HPMC K4M Prem. CR

X₂: Level of Lactose Spray Dried

X₃: level of AVICEL® PH 200

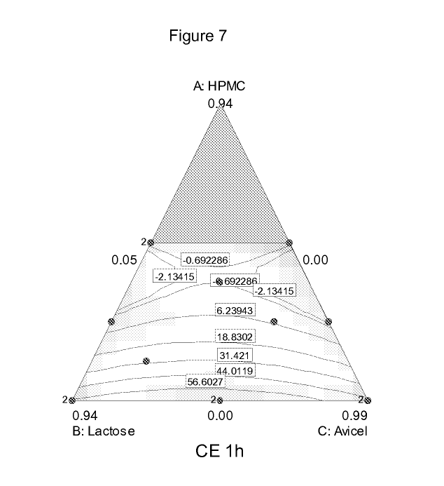

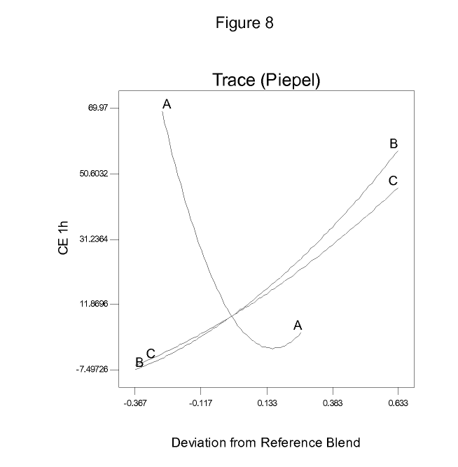

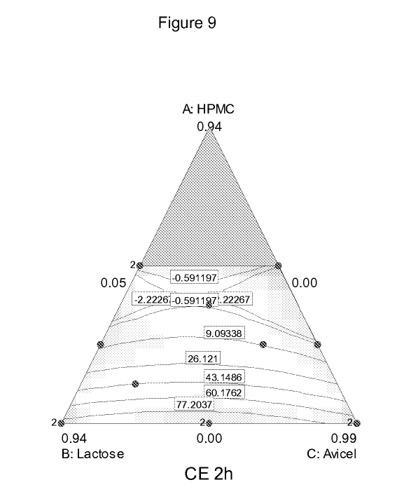

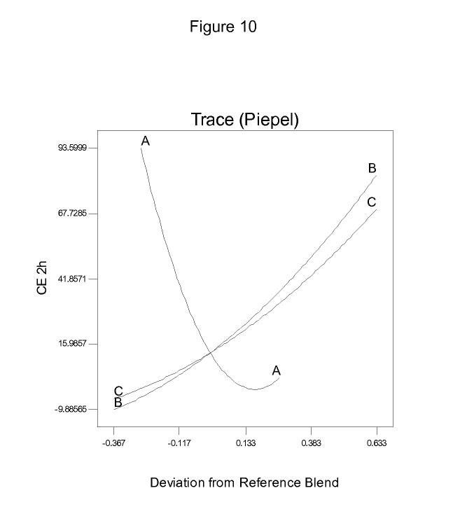

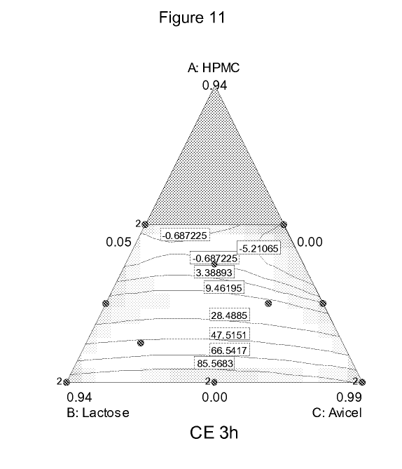

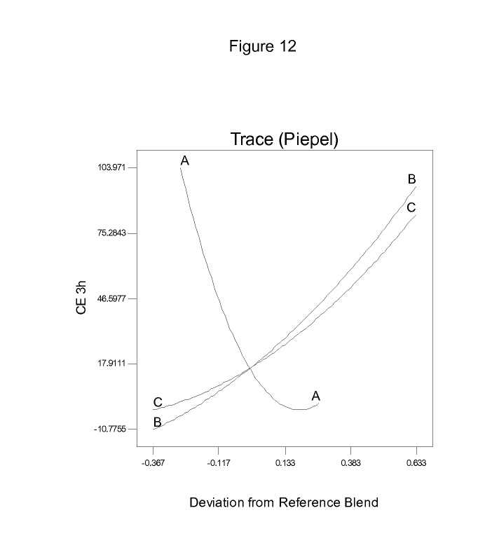

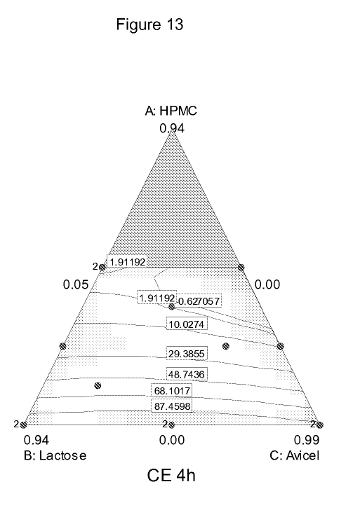

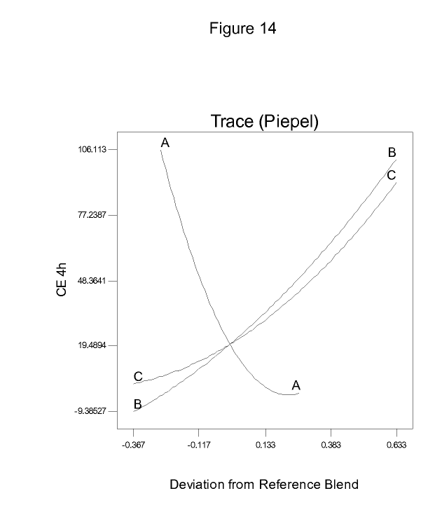

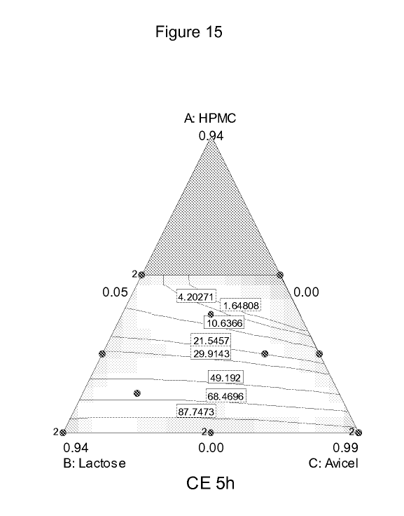

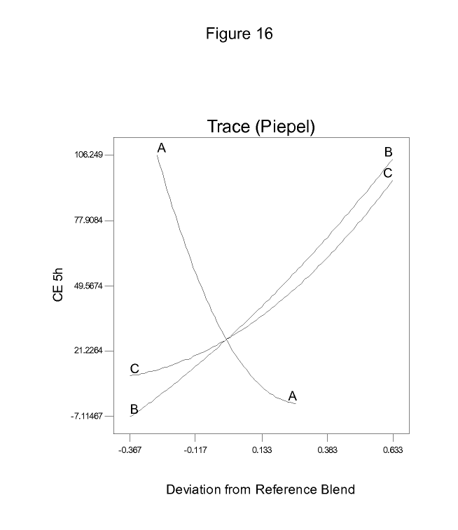

In order to evaluate the effect of HPMC levels in MPA external layer on the dissolution pattern of CE and MPA, the factors and response variables were related using polynomial equation with statistical analysis. As shown in Table 24, the approximations of response values of CE (Y_{CE 1h}, Y_{CE 2h}, Y_{CE 3h}, Y_{CE 4h}, and Y_{CE 5h}) based on the quadratic model were most suitable since it exhibits low standard deviation (ST), high R² values, a low PRESS, a reasonable agreement between predicted R² versus adjusted R². In addition, the adequate precision for all time points is more than 4. Table 25 lists all the coefficients for optimal regression equation for CE dissolution based on the quadratic model. FIGS. 7-16 illustrate the influence of levels of HPMC in the MPA outer tablet layer on the dissolution rate of CE from the tablet-in-tablet composition. For mixture designs, the trace plot shows the effects of changing each component along an imaginary line from the reference blend (defaulted to the overall centroid) to the vertex. As the amount of this component increases, the amounts of other component decreases, but their ratio to one another remains constant. On the trace plot, a steep slope or curvature in an input variable indicates a relatively high sensitivity of response. From these figures it can be concluded that HPMC (X₁) in MPA outer tablet layer was the main retardant for the CE dissolution from the tablet-in-tablet composition. The trace plots also indicate that both lactose (X₂) and AVICEL® (X₃) can increase the release rate of CE and the enhancement effect of lactose was higher than AVICEL® since the slope of the trace plot of lactose is higher than that of AVICEL®. This result might contribute to the water-soluble material, lactose, can stimulate the water penetration into the inner parts of the tablet-in-tablet, thus resulting in drug release from tablet-in-tablet.

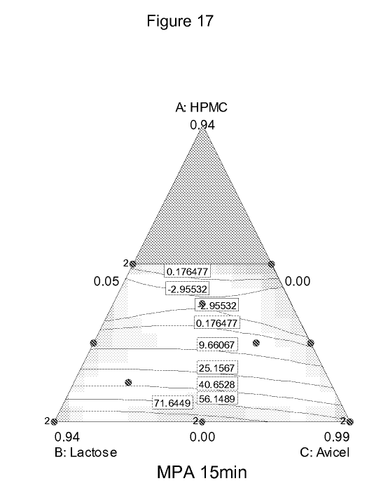

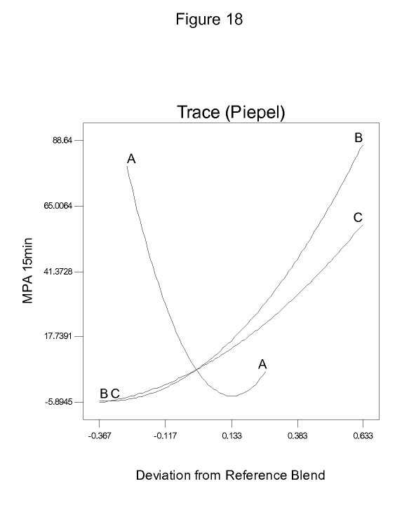

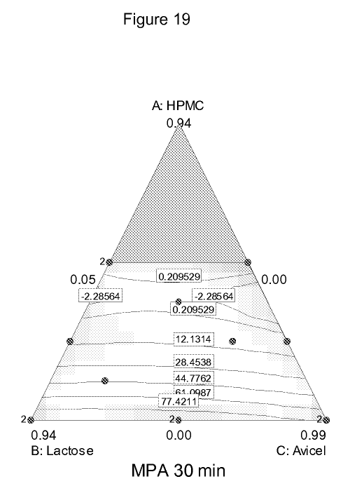

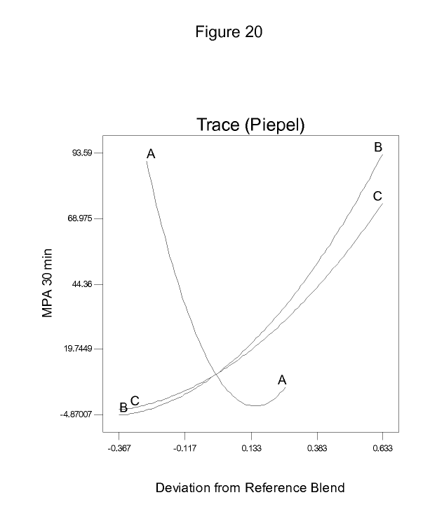

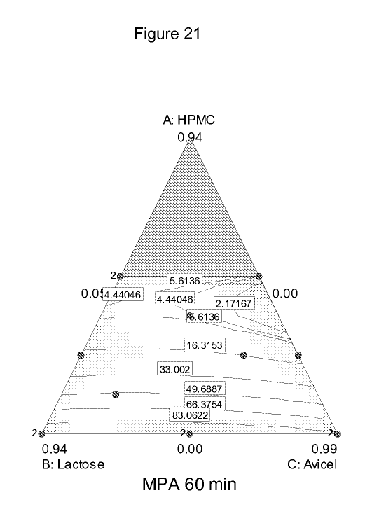

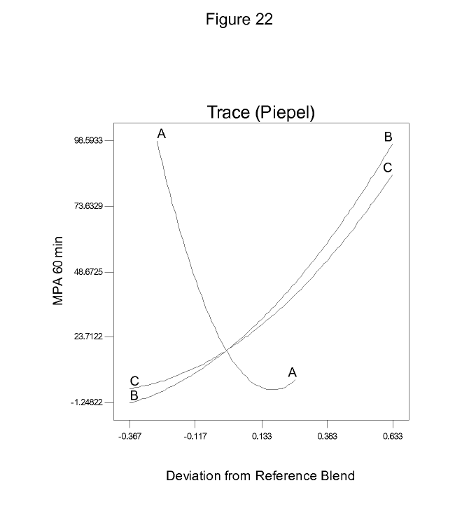

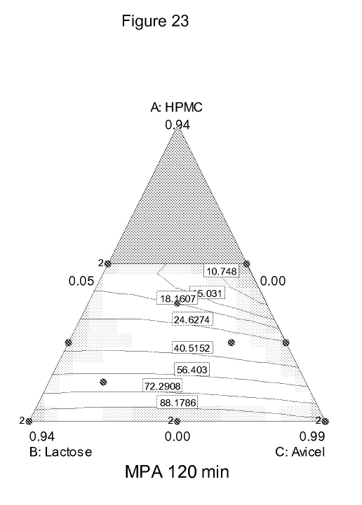

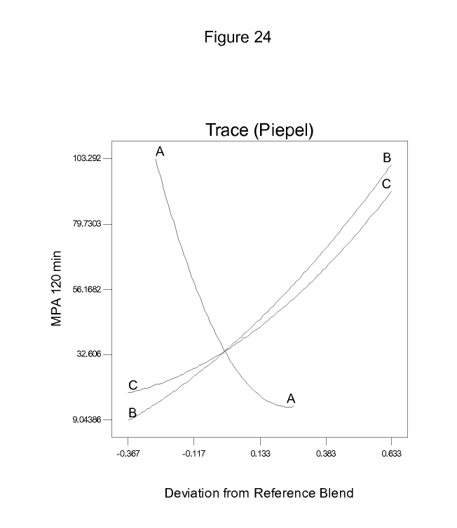

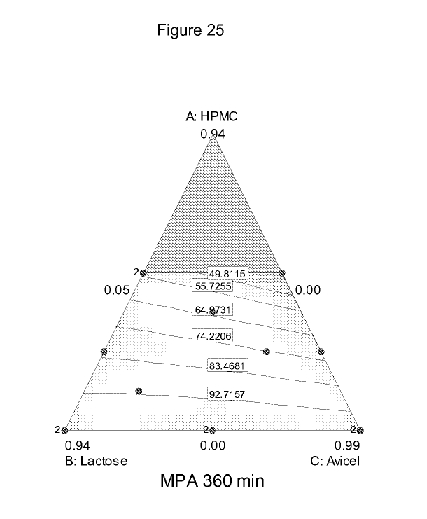

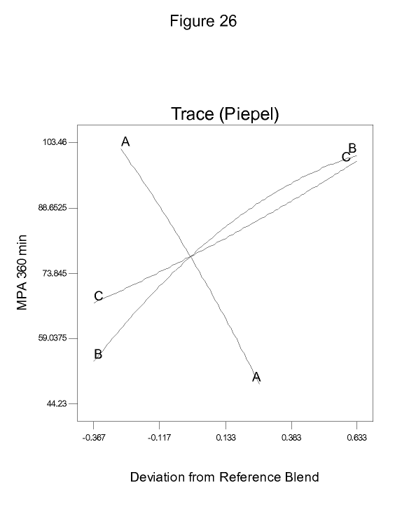

Table 26 displays the statistical parameters for MPA release rate. The results shown that approximations of response values of MPA (Y_{MPA 15 min}, Y_{MPA 30min}, Y_{MPA 60min}, Y_{MPA 120min}, and Y_{MPA 360min}) based on the quadratic model were the best fit since it exhibits low standard deviation (ST), high R² values and a low PRESS. Table 27 shows all the coefficients for optimal regression equation for dissolution rate of MPA from the tablet-in-tablet compositions. FIGS. 17-26 illustrate the influence of levels of HPMC in the MPA outer tablet layer on the dissolution rate of MPA. From these figures it can be concluded that similar to CE, HPMC (X₁) in MPA external layer was the main retardant for the MPA dissolution from the tablet-in-tablet. The trace plots also indicate that both lactose (X₂) and AVICEL® (X₃) can increase the release rate of MPA and the enhancement effect of lactose was higher than AVICEL® since the slope of trace plot of lactose is higher than that of AVICEL®.

### Stability Evaluation of CE/MPA Tablet-In-Tablets

One batch of CE/MPA tablet-in-tablet was evaluated for stability. The compositions for the CE core tablet as well as the MPA outer tablet layer are displayed in Tables 28 and 29. This batch was coated with Opadry® White (Colorcon, Inc., West Point, Pa.) with 2.8% weight gain using the Vector Coater LDCS 3 with 1.3 liter pan insert. The coated tablets were polished with carnauba wax. Fifty coated tablets were packed into 60 mL high-density polyethylene (HDPE) bottles and induction sealed. The sealed bottles were placed on stability at 40° C./75% RH and 30° C./60% RH conditions up to 6 months. The results of this study are shown in Tables 30 through 32. The dissolution at the initial time point was carried out on the uncoated tablet-in-tablet composition. As seen in the tables, this formulation is chemically stable under the conditions studied (30° C./60% RH and 40° C./75% RH) for up to 6 months without desiccant.

The same procedure was followed in another stability study, except that 1.0 g of the silica gel desiccant STRIPPAX® (Multisorb Technologies, Buffalo, N.Y.) was included in the 60 mL HDPE bottles with the CE/MPA tablet-in-tablet compositions. The formulations for the CE core and MPA outer layer are listed in Tables 33 and 34. The sealed bottles were placed on stability at 40° C./75% RH and 25° C./60% RH conditions up to 6 months and 12 months, respectively. Results are summarized in Table 35. The dessicant improved the stability of the tablet-in-tablet composition.

### Olfactory Screening of Tablet-in-tablets

As discussed above, CE has a characteristic odor that is generally not desirable in a tablet to be taken orally. To test the olfactory characteristics of tablet-in-tablet formulations described herein, olfactory screening was carried out on coated and uncoated tablet-in-tablet compositions. Table 36 displays the results. No characteristic odor of conjugated estrogen from pregnant mare urine was detected at 25° C./60% RH within the period of study for either the coated or uncoated tablet-in-tablet. Even under high temperature and humidity (40° C./75% RH) conditions, only uncoated tablets had an odor, which was very light, at the 4-week time point.

This illustrates that the formulation and the manufacturing procedures for the described tablet-in-tablet composition is robust and reproducible to produce conjugated estrogens/MPA tablet-in-tablet compositions with excellent weight variation and content uniformity. Furthermore, for the same external layer, the higher the content of the polymer in the CE core tablet portion, the slower the dissolution rate of CE. On the other hand, the dissolution rate of MPA from the outer tablet layer is not affected by the concentration of HPMC K100M CR in the CE core tablet portion.

The statistical experimental design study shows that a high level of polymer in the MPA layer will slow down the dissolution rate of MPA from the tablet-in-tablet. Both lactose monohydrate spray dried and AVICEL® can increase the release rate of MPA. The enhancement effect of lactose monohydrate spray dried was higher than AVICEL®. Similar to MPA, HPMC in the MPA outer tablet layer was the main retardant for the CE dissolution from the tablet-in-tablet composition with the same CE core tablet. Both lactose monohydrate spray dried and AVICEL® can increase the release rate of CE and the enhancement effect of lactose monohydrate spray dried was higher than AVICEL®. Without being limited to any particular theory, the greater dissolution of lactose monohydrate spray dried compared to AVICEL® may result from greater water solubility. Therefore, changing levels of HPMC in the MPA outer tablet layer portion and/or the CE core tablet portion can influence release rates of CE as well as those of MPA for this dosage form.

In addition to a robust formulation with multiple potential in vitro characteristics, the covered ranges for both CE, MPA and combinations of CE/MPA allow for a host of in vivo relationships in order to obtain a desired in vivo effect. This is a novel approach for obtaining a new robust formulation/process standpoint with acceptable stability characteristics that eliminates the need for sugar coating technology. This approach can be applied to other drug combinations, such as CE/BZA, to achieve optimum therapeutic effects.

TABLE 19
Weight Variation Results for CE/MPA Tablet-in-Tablet Compositions

Content Uniformity (%)

| Batch # | MPA | CE | Weight Variation (%) |
|---|---|---|---|
| Example 5 | 1.00 | 3.19 | 1.09 |
| Example 6 | 1.16 | 1.71 | 0.94 |
| Example 7 | 0.90 | 2.48 | 0.73 |

TABLE 20
Dissolution of MPA from Examples 5, 6, and 7

| Time (hrs) | Example 5 | Example 6 | Example 7 |
|---|---|---|---|
| 0 | 0 | 0 | 0 |
| 0.25 | 5 ± 1.1 | 6 ± 0.8 | 8 ± 0.6 |
| 0.50 | 8 ± 1.8 | 8 ± 1.2 | 11 ± 0.6 |
| 0.75 | 11 ± 2.2 | 11 ± 1.6 | 15 ± 0.7 |
| 1 | 15 ± 2.7 | 14 ± 2.1 | 18 ± 0.7 |
| 2 | 30 ± 4.7 | 30 ± 4.0 | 33 ± 1.4 |
| 6 | 84 ± 6.3 | 84 ± 4.5 | 86 ± 1.9 |
| 12 | 101 ± 2.0 | 100 ± 0.7 | 103 ± 1.6 |

Note:
Results presented are % released ± s.d. (n = 6).

TABLE 21
Dissolution of Conjugated Estrogens from Examples 5, 6, and 7

| Time (hrs) | Example 5 | Example 6 | Example 7 |
|---|---|---|---|
| 0 | 0 | 0 | 0 |
| 2 | 3 ± 1.6 | 7 ± 2.4 | 16 ± 10.9 |
| 5 | 29 ± 5.6 | 38 ± 2.9 | 55 ± 12.1 |
| 8 | 58 ± 9.4 | 71 ± 3.5 | 87 ± 7.4 |

Note:
Results presented are % released ± s.d. (n = 6).

TABLE 22
Dissolution Profiles of MPA from Examples 8-21

| | Percent Released at Different Time Intervals (% ± sd, n = 6) | | | | | | | |
|---|---|---|---|---|---|---|---|---|
| Example # | 0 min | 15 min | 30 min | 45 min | 60 min | 120 min | 360 min | 720 min |
| 8 | 0 | 82 ± 6.5 | 92 ± 5.4 | 96 ± 4.4 | 98 ± 3.5 | 101 ± 2.3 | 103 ± 1.3 | 103 ± 1.3 |
| 9 | 0 | 6 ± 0.5 | 9 ± 0.8 | 12 ± 1.1 | 16 ± 1.1 | 30 ± 2.2 | 82 ± 7.8 | 99 ± 1.7 |
| 10 | 0 | 55 ± 5.8 | 73 ± 5.0 | 83 ± 4.9 | 87 ± 3.6 | 94 ± 2.9 | 101 ± 1.9 | 102 ± 1.5 |
| 11 | 0 | 89 ± 4.6 | 94 ± 3.3 | 96 ± 3.0 | 96 ± 2.6 | 98 ± 2.0 | 99 ± 1.0 | 99 ± 1.0 |
| 12 | 0 | 80 ± 6.6 | 90 ± 3.9 | 93 ± 2.8 | 95 ± 1.9 | 97 ± 1.4 | 98 ± 0.9 | 97 ± 0.9 |
| 13 | 0 | 3 ± 0.2 | 4 ± 0.2 | 6 ± 0.4 | 7 ± 0.5 | 16 ± 1.1 | 55 ± 3.7 | 95 ± 2.7 |
| 14 | 0 | 2 ± 0.2 | 3 ± 0.4 | 5 ± 0.6 | 7 ± 0.9 | 14 ± 2.0 | 44 ± 6.3 | 83 ± 8.9 |
| 15 | 0 | 2 ± 0.2 | 3 ± 0.3 | 5 ± 0.5 | 7 ± 0.8 | 15 ± 1.3 | 53 ± 2.1 | 94 ± 2.6 |
| 16 | 0 | 28 ± 5.7 | 42 ± 4.4 | 54 ± 3.6 | 63 ± 3.5 | 87 ± 5.2 | 99 ± 2.1 | 100 ± 0.8 |
| 17 | 0 | 60 ± 7.1 | 76 ± 6.5 | 83 ± 5.7 | 87 ± 5.1 | 93 ± 4.1 | 99 ± 2.2 | 99 ± 1.5 |
| 18 | 0 | 2 ± 0.3 | 4 ± 0.6 | 6 ± 0.8 | 9 ± 1.2 | 21 ± 2.1 | 74 ± 3.8 | 101 ± 1.5 |
| 19 | 0 | 2 ± 0.3 | 5 ± 0.4 | 7 ± 0.5 | 10 ± 0.7 | 21 ± 1.5 | 68 ± 4.0 | 99 ± 1.2 |
| 20 | 0 | 2 ± 0.1 | 4 ± 0.3 | 6 ± 0.6 | 8 ± 0.8 | 18 ± 1.8 | 62 ± 3.1 | 98 ± 2.3 |
| 21 | 0 | 88 ± 4.5 | 93 ± 3.4 | 95 ± 3.0 | 95 ± 2.5 | 98 ± 1.4 | 100 ± 0.6 | 100 ± 1.0 |

TABLE 23
Dissolution Profiles of Conjugated Estrogen from Examples 8-21

| | Percent Released at Different Time Intervals (% ± sd, n = 6) | | | | | | |
|---|---|---|---|---|---|---|---|
| Example # | 0 hr | 1 hr | 2 hr | 3 hr | 4 hr | 5 hr | 8 hr |
| 8 | 0 | 67 ± 7.7 | 90 ± 5.7 | 99 ± 2.3 | 100 ± 2.5 | 101 ± 1.0 | 100 ± 2.0 |
| 9 | 0 | 0.2 ± 0.4 | 2 ± 1.5 | 7 ± 2.5 | 16 ± 3.7 | 28 ± 4.9 | 69 ± 6.1 |
| 10 | 0 | 48 ± 6.8 | 73 ± 5.8 | 88 ± 6.0 | 97 ± 4.4 | 101 ± 3.2 | 102 ± 1.8 |
| 11 | 0 | 58 ± 11.0 | 83 ± 9.5 | 94 ± 6.1 | 99 ± 3.1 | 101 ± 3.1 | 100 ± 2.2 |
| 12 | 0 | 70 ± 7.5 | 92 ± 4.4 | 100 ± 1.6 | 101 ± 1.3 | 101 ± 1.6 | 101 ± 1.1 |
| 13 | 0 | 0 ± 0 | 0 ± 0 | 0 ± 0 | 0.1 ± 0.2 | 2 ± 0.5 | 20 ± 2.1 |
| 14 | 0 | 0 ± 0 | 0 ± 0 | 0 ± 0 | 0 ± 0 | 0 ± 0 | 5 ± 0.8 |
| 15 | 0 | 0 ± 0 | 0 ± 0 | 0 ± 0 | 0 ± 0 | 2 ± 0.7 | 19 ± 2.1 |
| 16 | 0 | 33 ± 4.6 | 58 ± 9.2 | 78 ± 9.0 | 89 ± 7.6 | 94 ± 3.8 | 100 ± 2.7 |
| 17 | 0 | 44 ± 5.8 | 68 ± 7.0 | 84 ± 7.3 | 93 ± 6.4 | 96 ± 4.9 | 97 ± 3.0 |
| 18 | 0 | 0 ± 0 | 0 ± 0 | 0 ± 0 | 3 ± 0.6 | 9 ± 0.9 | 40 ± 1.4 |
| 19 | 0 | 0 ± 0 | 0.1 ± 0.2 | 1 ± 0.6 | 3 ± 1.5 | 6 ± 2.9 | 28 ± 6.8 |
| 20 | 0 | 0 ± 0 | 0 ± 0 | 0 ± 0 | 2 ± 0.7 | 5 ± 1.6 | 27.8 ± 4.4 |
| 21 | 0 | 55 ± 4.4 | 79 ± 4.1 | 91 ± 2.7 | 96 ± 1.7 | 99 ± 1.3 | 99 ± 1.5 |

TABLE 24
Optimal Regression Equation for Each Response Variable for CE Dissolution

| Model | Coefficient | CE % 1 h | CE % 2 h | CE % 3 h | CE % 4 h | CE % 5 h |
|---|---|---|---|---|---|---|
| linear | Std. Dev. | 14 | 18 | 20 | 20 | 18 |
| | R-Square | 0.8063 | 0.8318 | 0.8432 | 0.8615 | 0.8807 |
| | Adjusted R-Square | 0.771 | 0.8012 | 0.8147 | 0.8364 | 0.859 |
| | Predicted R-Square | 0.7161 | 0.7509 | 0.7668 | 0.7932 | 0.8206 |
| | PRESS | 3162 | 5436 | 6631 | 6333 | 5351 |
| | Adeq. Precision | 11 | 12 | 12 | 13 | 15 |
| Quadratic | Std. Dev. | 3 | 7 | 11 | 14 | 14 |
| | R-Square | 0.9942 | 0.9828 | 0.963 | 0.9505 | 0.9476 |
| | Adjusted R-Square | 0.9906 | 0.9721 | 0.9399 | 0.9195 | 0.9149 |
| | Predicted R-Square | 0.9861 | 0.9614 | 0.9095 | 0.8662 | 0.838 |
| | PRESS | 155 | 843 | 2574 | 4097 | 4831 |
| | Adeq. Precision | 38 | 21 | 15 | 13 | 13 |
| Special | Std. Dev. | 3 | 7 | 12 | 15 | 15 |
| Cubic | R-Square | 0.9944 | 0.9831 | 0.9639 | 0.9517 | 0.9486 |
| | Adjusted R-Square | 0.9895 | 0.9686 | 0.933 | 0.9103 | 0.9045 |
| | Predicted R-Square | 0.9813 | 0.9552 | 0.8967 | 0.8461 | 0.8098 |
| | PRESS | 209 | 978 | 2936 | 4713 | 5672 |
| | Adeq. Precision | 33 | 19 | 13 | 11 | 11 |

TABLE 25
Optimal Regression Equation Coefficients for CE dissolution

| Coefficient | Y_{CE 1 h} | Y_{CE 2 h} | Y_{CE 3 h} | Y_{CE 4 h} | Y_{CE 5 h} |
|---|---|---|---|---|---|
| b₁X₁ | 157.4 | 193.09 | 184.1 | 146.45 | 100.25 |
| b₂X₂ | 54.47 | 80.09 | 93.71 | 101.05 | 104.11 |
| b₃X₃ | 46.75 | 69.86 | 84.19 | 92.12 | 95.89 |
| b₁₂X₁X₂ | −437.12 | −557.91 | −561.48 | −489.08 | −383.44 |
| b₁₃X₁X₃ | −414.17 | −542.65 | −569.13 | −518.63 | −441.05 |
| b₂₃X₂X₃ | 76.74 | 79.7 | 65.43 | 43.23 | 29.91 |

TABLE 26
Optimal Regression Equation for Each Response Variable for MPA Dissolution

| | | MPA % | | | | |
|---|---|---|---|---|---|---|
| Model | Coefficient | 15 min | MPA % 30 min | MPA % 60 min | MPA % 120 min | MPA % 360 min |
| Linear | Std. Dev. | 18 | 18 | 17 | 15 | 5 |
| | R-Square | 0.8131 | 0.8393 | 0.8629 | 0.8843 | 0.9612 |
| | Adjusted R- | 0.7791 | 0.8101 | 0.838 | 0.8633 | 0.9542 |
| | Square | | | | | |
| | Predicted R- | 0.719 | 0.7576 | 0.7922 | 0.8276 | 0.937 |
| | Square | | | | | |
| | PRESS | 5162 | 5323 | 4828 | 3557 | 371 |
| | Adeq. Precision | 12 | 12 | 13 | 15 | 26 |
| Quadratic | Std. Dev. | 5 | 3 | 5 | 10 | 4 |
| | R-Square | 0.991 | 0.9975 | 0.9896 | 0.962 | 0.9832 |
| | Adjusted R- | 0.9853 | 0.996 | 0.9831 | 0.9383 | 0.9727 |
| | Square | | | | | |
| | Predicted R- | 0.9698 | 0.9943 | 0.9757 | 0.9032 | 0.9411 |
| | Square | | | | | |
| | PRESS | 555 | 126 | 565 | 1997 | 347 |
| | Adeq. Precision | 30 | 55 | 26 | 15 | 26 |
| Special | Std. Dev. | 5 | 3 | 6 | 10 | 4 |
| Cubic | R-Square | 0.991 | 0.9977 | 0.9903 | 0.9633 | 0.9836 |
| | Adjusted R- | 0.9832 | 0.9958 | 0.982 | 0.9318 | 0.9696 |
| | Square | | | | | |
| | Predicted R- | 0.9553 | 0.9893 | 0.9725 | 0.8893 | 0.9246 |
| | Square | | | | | |
| | PRESS | 822 | 234 | 639 | 2284 | 445 |
| | Adeq. Precision | 26 | 49 | 24 | 13 | 23 |

TABLE 27
Optimal Regression Equation Coefficients for MPA dissolution

| Coefficient | Y_{MPA 15 min} | Y_{MPA 30 min} | Y_{MPA 60 min} | Y_{MPA 120 min} | Y_{MPA 360 min} |
|---|---|---|---|---|---|
| b₁X₁ | 217.8 | 218.36 | 188.75 | 121.23 | −21.48 |
| b₂X₂ | 87.91 | 93.12 | 96.98 | 100.52 | 100.91 |
| b₃X₃ | 58.83 | 75.08 | 86.32 | 92.04 | 99.99 |
| b₁₂X₁X₂ | −616.98 | −617.39 | −545.68 | −377.76 | 69.72 |
| b₁₃X₁X₃ | −536.63 | −576.95 | −540.35 | −397.91 | 12.22 |
| b₂₃X₂X₃ | 30.77 | 31.94 | 32.68 | 32.91 | 9.65 |

TABLE 28
Composition of Conjugated Estrogens Internal
Tablet Portion For Stability Evaluation

| Description | Input/Tablet (mg) | % W/W |
|---|---|---|
| CE Desiccation with Lactose at 42.9 mg/g | 10.4895 | 8.74 |
| Lactose Monohydrate Spray Dried | 67.2105 | 56.01 |
| Avicel PH 101, NF | 18 | 15.00 |
| HPMC K100M Premium CR | 24 | 20.00 |
| Magnesium Stearate, NF | 0.3 | 0.25 |

TABLE 29
Composition of MPA External Tablet Portion For Stability Evaluation

| | Input/Tablet | |
|---|---|---|
| Description | (mg) | % W/W |
| Medroxyprogesterone Acetate, USP, Micronized | 1.5 | 0.63 |
| @100% | | |
| Lactose Monohydrate Spray Dried, NF | 93.9 | 39.13 |
| Microcrystalline Cellulose, NF (Avicel PH 200) | 96 | 40.00 |
| HPMC K4M Premium CR | 48 | 20.00 |
| Magnesium Stearate, NF | 0.6 | 0.25 |

TABLE 30
Dissolution Profiles of MPA from Conjugated Estrogens/MPA Tablet-in-Tablet
(with 20% HPMC in Internal Tablet Portion) Upon Stability

| | Percent Release of MPA (% ± sd, n = 6) | | | | | | |
|---|---|---|---|---|---|---|---|
| Time | Time | 1 MO | 1 MO | 3 MO | 3 MO | 6 MO | 6 MO |
| (hr) | Zero* | (30° C./60RH) | (40° C./75RH) | (30° C./60RH) | (40° C./75RH) | (30° C./60RH) | (40° C./75RH) |
| 0.25 | 6 ± 0.8 | 2 ± 0.6 | 2 ± 0.2 | 4 ± 0.6 | 4 ± 1.5 | 3 ± 0.4 | 3 ± 0.3 |
| 0.5 | 8 ± 1.2 | 5 ± 1.1 | 5 ± 0.5 | 6 ± 0.9 | 7 ± 2.1 | 5 ± 0.6 | 5 ± 0.5 |
| 0.75 | 11 ± 1.6 | 7 ± 1.4 | 8 ± 1.0 | 9 ± 1.2 | 10 ± 2.5 | 8 ± 0.9 | 8 ± 0.8 |
| 1 | 14 ± 2.1 | 11 ± 1.8 | 11 ± 1.5 | 12 ± 1.4 | 14 ± 2.9 | 11 ± 1.3 | 11 ± 1.1 |
| 2 | 30 ± 4.0 | 24 ± 4.2 | 26 ± 4.0 | 24 ± 3.0 | 30 ± 4.1 | 25 ± 3.5 | 26 ± 2.4 |
| 6 | 84 ± 4.5 | 75 ± 7.2 | 77 ± 6.2 | 76 ± 3.8 | 85 ± 2.7 | 76 ± 6.2 | 79 ± 5.2 |
| 12 | 100 ± 0.7 | 95 ± 1.4 | 95 ± 1.9 | 96 ± 2.3 | 98 ± 1.2 | 97 ± 2.2 | 95 ± 0.9 |

Note:
*the dissolution was carried out on the uncoated tablet-in-tablet.

TABLE 31
Dissolution Profile of CE from Conjugated Estrogens/MPA Tablet-in-Tablet
(with 20% HPMC in Internal Tablet Portion) Upon Stability

| | Percent Release of CE (% ± sd, n = 6) | | | | | | |
|---|---|---|---|---|---|---|---|
| Time | Time | 1 MO | 1 MO | 3 MO | 3 MO | 6 MO | 6 MO |
| (hr) | Zero* | (30° C./60RH) | (40° C./75RH) | (30° C./60RH) | (40° C./75RH) | (30° C./60RH) | (40° C./75RH) |
| 2 | 7 ± 2.4 | 4 ± 2.4 | 1 ± 0.7 | 3 ± 2.5 | 1 ± 1.1 | 4 ± 3.2 | 2 ± 1.8 |
| 5 | 38 ± 2.9 | 29 ± 6.8 | 20 ± 4.7 | 28 ± 7.8 | 26 ± 4.7 | 27 ± 5.8 | 21 ± 4.5 |
| 8 | 71 ± 3.5 | 62 ± 8.0 | 51 ± 7.5 | 61 ± 7.7 | 60 ± 5.8 | 60 ± 5.1 | 54 ± 5.9 |

Note:
*the dissolution was carried out on the uncoated tablet-in-tablet.

TABLE 32
Stability Results for Conjugated Estrogens/MPA Tablet-in-Tablet Composition

| | | | 3 MO | 3 MO | 6 MO | 6 MO |
|---|---|---|---|---|---|---|
| Parameters | | Time Zero | (30° C./60RH) | (40° C./75RH) | (30° C./60RH) | (40° C./75RH) |
| Potency (%) | CE | 100.08 | 100.70 | 96.91 | 102.11% | 91.58% |
| | MPA | 97.00 | 96.60 | 96.30 | 94.53% | 94.23% |

| | | | | | |
|---|---|---|---|---|---|
| Water KF (%) | 5.40 | 4.79 | 4.78 | 4.44% | 4.71% |

TABLE 33
Composition of Conjugated Estrogens Internal Tablet
Portion For Stability Evaluation with Dessicant

| Ingredient | input/tablet (mg) | w/w % |
|---|---|---|
| CE desiccation with lactose | 10.4895 | 8.74 |
| Lactose monohydrate spray dried | 79.2105 | 66.01 |
| Avicel PH 101 | 18 | 15.00 |
| HPMC 2208 (K100M) Premium CR | 12 | 10.00 |
| Magnesium Stearate | 0.3 | 0.25 |
| Total | 120 | 100.00 |

TABLE 34
Composition of MPA External Tablet Portion For
Stability Evaluation with Dessicant

| Ingredient | input/tablet (mg) | w/w % |
|---|---|---|
| Medroxyprogesterone Acetate, USP, | 1.5 | 0.625 |
| Micronized @100% | | |
| Lactose monohydrate spray dried | 86.7 | 36.13 |
| Avicel PH 200 | 110.4 | 46.00 |
| HPMC K4M Premium CR | 40.8 | 17.00 |
| Magnesium Stearate | 0.6 | 0.25 |
| Total | 240.00 | 100.0 |

TABLE 35
Stability Results for Conjugated Estrogens/MPA Tablet-in-Tablet Composition Stored
with Desiccant

| | | Time | 3 MO | 3 MO | 6 MO | 6 MO | 12 MO |
|---|---|---|---|---|---|---|---|
| Parameters | | Zero | (25° C./60RH) | (40° C./75RH) | (25° C./60RH) | (40° C./75RH) | (25° C./60RH) |
| Potency | CE | 104.22 | 103.8 | 104.11 | 103.78 | 100.05 | Potency (%) |
| (%) | MPA | 96.40 | 96.5 | 96.5 | 96.5 | 95.7 | |

| | | | | | | |
|---|---|---|---|---|---|---|
| Water KF (%) | 5.45 | 4.24 | 4.39 | 3.78 | 4.08 | 4.56 |

TABLE 36
Olfactory Screening of Conjugated Estrogens/MPA Tablet-in-Tablet
Uncoated and Coated with 3% Opadry White

| | 25° C./60% RH | | 40° C./75% RH | |
|---|---|---|---|---|
| Duration (week) | Uncoated | Coated | Uncoated | Coated |
| 1 | − | − | − | − |
| 2 | − | − | − | − |
| 3 | − | − | − | − |
| 4 | − | − | +/− | − |
| 5 | − | − | − | − |
| 6 | − | − | − | − |
| 7 | − | − | − | − |
| 8 | − | − | − | − |
| 9 | − | − | − | − |
| 10 | − | − | +/− | +/− |
| 11 | − | − | − | − |
| 12 | − | − | − | − |
| 13 | − | − | +/− | − |
| 14 | − | − | − | − |
| 15 | − | − | − | − |
| 16 | − | − | +/− | − |

Note:
40 tablets are packed into 40 ml HDPE bottle.
−: no change
+: slightly changed (The severity of odor will be expressed by the number of +)
+/−: slight odor as the cap removed. No odor when reopen the cap immediately

### Examples 23-25

Certain tablet-in-tablet compositions described herein have an outer layer of bazedoxifene. Examples 23-25 describe methods of making such compositions with varying amounts of AVICEL®, HPMC, and lactose monohydrate spray dried.

### Example 23

### Bazedoxifene Acetate Dry Granulation with 5% HPMC

Using the ingredient amounts in Table 37, the dry granulation was completed using an Alexanderwerk WP 120×40 Roller Compactor followed the procedures below for a batch size of 1 kg:

1. AVICEL® PH 200 (FMC Biopolymer, Philadelphia, Pa.) and BZA (Berlichem, Inc., Fairfield, N.J.) were passed through a #30 mesh screen.
2. The ingredients were blended together in a 4 Qt V-blender for approximately 5 minutes at about 22 rpm.
3. Lactose monohydrate spray dried (Foremost Farms USA, Baraboo, Wis.) and HPMC K100M Premium CR (Dow Chemical Co., Midland, Mich.) were added into the blender and blended for approximately 15 minutes at about 22 rpm.
4. MS was screened with about 100 g of the blended material through the #30 mesh screen.
5. The mixture of step 4 was added into the blender and blended for approximately 3 minutes at about 22 rpm.
6. The blend of step 5 was granulated using Alexanderwerk WP 120×40 Roller Compactor at following parameters:

Screen Feeder Speed: 55 rpm

Roller Speed: 7 rpm

Fine Granulator Speed: 60 rpm

Hydraulic Pressure: 40 bar

Roller Gap: 1.5 mm.

Vacuum: on

TABLE 37
Composition of BZA Granulation with 5% HPMC K100M CR

| Ingredients | mg/tab | w/w % |
|---|---|---|
| BZA Micronized (A) | 22.58 | 7.51 |
| Lactose monohydrate spray dried | 138.85 | 46.17 |
| Avicel PH 200 | 122.49 | 40.73 |
| HPMC K100M Premium CR | 15.31 | 5.09 |
| Magnesium Stearate | 1.5 | 0.50 |
| total | 300.73 | 100.0 |

Note:
(A) dosed as free base. Quantity is adjusted based on the actual potency.

### Example 24

### Bazedoxifene Acetate Dry Granulation with 10% HPMC

Using the ingredient amounts in Table 38, a bazedoxifene acetate dry granulation was prepared by following the procedure in Example 23.

TABLE 38
Composition of BZA Granulation with 10% HPMC K100M CR

| Ingredients | mg/tab | w/w % |
|---|---|---|
| BZA Micronized (A) | 22.58 | 7.51 |
| Lactose monohydrate spray dried | 123.54 | 41.08 |
| Avicel PH 200 | 122.49 | 40.73 |
| HPMC K100M Premium CR | 30.62 | 10.18 |
| Magnesium Stearate | 1.5 | 0.50 |
| total | 300.73 | 100.0 |

Note:
(A) dosed as free base. Quantity is adjusted based on the actual potency.

### Example 25

### Bazedoxifene Acetate Dry Granulation with 20% HPMC

Using the ingredient amounts in Table 39, a bazedoxifene acetate dry granulation was prepared by following the procedure in Example 23.

TABLE 39
Composition of BZA Granulation with 20% HPMC K100M CR

| Ingredients | mg/tab | w/w % |
|---|---|---|
| BZA Micronized (A) | 22.58 | 7.51 |
| Lactose monohydrate spray dried | 92.92 | 30.90 |
| Avicel PH 200 | 122.49 | 40.73 |
| HPMC K100M Premium CR | 61.24 | 20.36 |
| Magnesium Stearate | 1.5 | 0.50 |
| total | 300.73 | 100.0 |

Note:
(A) dosed as free base. Quantity is adjusted based on the actual potency.

### Examples 26-32

Certain tablet-in-tablet compositions described herein have an outer layer of bazedoxifene and one or more antioxidants. Examples 26-32 describe methods of making such compositions with varying amounts of antioxidants, AVICEL®, HPMC, and lactose monohydrate spray dried.

### Example 26

### Granulation of Bazedoxifene Acetate with Antioxidants

Using the ingredient amounts in Table 40, the dry granulation was completed using a Fitzpatrick Chilsonator IR 220 using the procedures below for a batch size of 1 kg:
1. AVICEL® PH 200 (FMC Biopolymer, Philadelphia, Pa.) and BZA were passed through a #30 mesh screen.
2. The ingredients were blended together in a 4 Qt V-blender for approximately 5 minutes at about 22 rpm.
3. Lactose monohydrate spray dried and HPMC were added into the blender and blended for approximately 15 minutes at about 22 rpm.
4. The intra-granular magnesium stearate was screened with about 100 g of the blended material through the #30 mesh screen.
5. The mixture of step 4 was added to the blender and blended for approximately 3 minutes at about 22 rpm.
6. The blend of step 2 was granulated using a Fitzpatrick Chilsonator IR 220 at following parameters:
   Roll Pressure: approximately 90-210 psi
   Roll Force: approximately 500-2500 lb/in
   Roll Speed: approximately 9 rpm
   VFS: approximately 150-200 rpm
   HFS: approximately 50-60 rpm
7. The ribbon was milled using a Quadro Comil 197S at about 20% motor speed using a screen with about a 1.575 mm opening.
8. The milled materials were weighed and blended in the 4 Qt V-blender for approximately 10 minutes at about 22 rpm.
9. The quantity of extra-granular MS needed was calculated based on the yield.
10. The MS was weighed and added to the blender and blended for approximately 3 minutes at about 22 rpm.

TABLE 40
Composition of BZA Granulation with 5% HPMC
K100M CR and Antioxidants

| Ingredients | mg/tab | w/w % |
|---|---|---|
| BZA Micronized (A) | 22.58 | 7.53 |
| Lactose monohydrate spray dried | 135.97 | 45.32 |
| Avicel PH 200 | 120 | 40.00 |
| HPMC K100M Premium CR | 15 | 5.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Example 27

### Granulation of Bazedoxifene Acetate with Antioxidants

Using the ingredient amounts in Table 41 the bazedoxifene acetate granulation was prepared by the procedure in Example 26.

TABLE 41
Composition of BZA Granulation with 10% HPMC
K100M CR and Antioxidants

| Ingredients | mg/tab | w/w % |
|---|---|---|
| BZA Micronized (A) | 22.58 | 7.53 |
| Lactose monohydrate spray dried | 120.97 | 40.32 |
| Avicel PH 200 | 120 | 40.00 |
| HPMC K100M Premium CR | 30 | 10.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Example 28

### Granulation of Bazedoxifene Acetate with Antioxidants

In some cases, it is desirable to formulate the outer tablet layer with one or more antioxidants. In one example of such a formulation, using the ingredient amounts in Table 42, the bazedoxifene acetate granulation was prepared by the procedure in Example 26.

TABLE 42
Composition of BZA Granulation with 20% HPMC
K100M CR and Antioxidants

| Ingredients | mg/tab | w/w % |
|---|---|---|
| BZA Micronized (A) | 22.58 | 7.53 |
| Lactose monohydrate spray dried | 90.97 | 30.32 |
| Avicel PH 200 | 120 | 40.00 |
| HPMC K100M Premium CR | 60 | 20.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Example 29

### Granulation of Bazedoxifene Acetate with Antioxidants

Using the ingredient amounts in Table 43, the bazedoxifene acetate granulation was prepared by the procedure in Example 26.

TABLE 43
Composition of BZA Granulation with 5% HPMC
K100 LV and Antioxidants

| Ingredients | mg/tab | w/w % |
|---|---|---|
| BZA Micronized (A) | 22.58 | 7.53 |
| Lactose monohydrate spray dried | 135.97 | 45.32 |
| Avicel PH 200 | 120 | 40.00 |
| HPMC K100 LV | 15 | 5.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Example 30

### Granulation of Bazedoxifene Acetate with Antioxidants

Using the ingredient amounts in Table 44, the bazedoxifene acetate granulation was prepared by the procedure in Example 26.

TABLE 44
Composition of BZA Granulation with 20% HPMC
K100 LV and Antioxidants

| Ingredients | mg/tab | w/w % |
|---|---|---|
| BZA Micronized (A) | 22.58 | 7.53 |
| Lactose monohydrate spray dried | 90.97 | 30.32 |
| Avicel PH 200 | 120 | 40.00 |
| HPMC K100 LV | 60 | 20.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Example 31

### Granulation of Bazedoxifene Acetate with Antioxidants

Using the ingredient amounts in Table 45, the bazedoxifene acetate granulation was prepared by the procedure in Example 26.

TABLE 45
Composition of BZA Granulation with 5% HPMC
K4M CR and Antioxidants

| Ingredients | mg/tab | w/w % |
|---|---|---|
| BZA Micronized (A) | 22.58 | 7.53 |
| Lactose monohydrate spray dried | 135.97 | 45.32 |
| Avicel PH 200 | 120 | 40.00 |
| HPMC K4M Premium CR | 15 | 5.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Example 32

### Granulation of Bazedoxifene Acetate with Antioxidants

Using the ingredient amounts in Table 46, the bazedoxifene acetate granulation was prepared by the procedure in Example 26.

TABLE 46
Composition of BZA Granulation with 20% HPMC
K4M CR and Antioxidants

| Ingredients | mg/tab | w/w % |
|---|---|---|
| BZA Micronized (A) | 22.58 | 7.53 |
| Lactose monohydrate spray dried | 90.97 | 30.32 |
| Avicel PH 200 | 120 | 40.00 |
| HPMC K4M Premium CR | 60 | 20.00 |
| Ascorbic Acid Fine Powder | 4.5 | 1.50 |
| Dry Vitamin E-Acetate 50% DC | 0.45 | 0.15 |
| Intra-granular Magnesium Stearate | 0.75 | 0.25 |
| Extra-granular Magnesium Stearate | 0.75 | 0.25 |
| total | 300.00 | 100.0 |

Note:
(A) dosed as free base. Quantity is adjusted based on the actual potency.

### Examples 33-35

Certain tablet-in-tablet compositions described herein contain a disintegrant in the outer tablet layer. The disintegrant provides nearly immediate release of API from the outer tablet layer. Examples 33-35 describe methods of making such tablet-in-tablet compositions.

### Example 33

### Preparation of BZA Immediate Release Formulation

The composition of BZA immediate release formulation is shown in Table 47. The following process was used to produce 500 g of this immediate release BZA granulation:

1. Intra-granular excipients were screened through a #20 mesh screen and blended in a 2 Qt V-blender for approximately 15 minutes at about 22 rpm.
2. The blend of step 1 was granulated using a Fitzpatrick roller compactor:

Roll Pressure: approximately 602 psi

Roll Force: approximately 5000 psi

Roll Speed: approximately 9 rpm

VFS: approximately 150 rpm

HFS: approximately 25 rpm

3. The ribbon was milled using a comil with a 2A screen at approximately 20% motor speed.
4. The intra-granular granulation was weighed. The extra-granular excipients needed were calculated based on the weight.
5. The intra-granular granulation of step 4 was placed into a V-blender and blended for approximately 10 minutes at about 22 rpm.
6. Lactose fast flow (Foremost Farms USA, Baraboo, Wis.), PROSOLV® (JRS Pharma, Patterson, N.Y.), starch pregelatin 1500 (Colorcon, West Point, Pa.), and EXPLOTAB® (JRS Pharma, Patterson, N.Y.) were screened through a #20 mesh and added to the blender. The mixture was then blended for about 10 minutes at approximately 22 rpm.
7. The magnesium stearate was screened through the same screen with about 100 g of the blend of step 6.
8. The mixture of step 7 was added to the blender and blended for about 3 minutes at approximately 22 rpm.

TABLE 47
Immediate Release Formulation of BZA External Layer

| | | |
|---|---|---|
| Ingredients | mg/tab | w/w % |

Intra-Granular

| | | |
|---|---|---|
| BZA Micronized (A) | 22.58 | 5.65 |
| Lactose Fast Flow | 103.82 | 25.96 |
| Ascorbic Acid Fine powder | 6 | 1.50 |
| Prosolv 90 | 100 | 25.00 |
| Lutrol Micro 68 | 6 | 1.50 |
| Explotab | 16 | 4.00 |

Extra-Granular

| | | |
|---|---|---|
| Lactose Fast Flow | 79.6 | 19.90 |
| Prosolv 90 | 40 | 10.00 |
| Starch Pregelatinized 1500 | 16 | 4.00 |
| Explotab | 8 | 2.00 |
| Magnesium Stearate | 2 | 0.50 |
| Total | 400 | 100 |

Note:
(A) dosed as free base. Quantity is adjusted based on the actual potency.

### Example 34

### Preparation of the Conjugated Estrogen Internal Tablet

The composition of CE with 10% HPMC K100M granulation is listed in Table 48. A CEDL at 42.9 mg/g mixture was granulated with all other ingredients using water in a high shear granulator followed by the procedures below for a batch size of 1.5 kg:

1. CEDL was mixed with lactose monohydrate spray dried, AVICEL® (FMC Biopolymer, Philadelphia, Pa.), and HPMC in a Collette shear mixer for approximately 5 minutes with plows at approximately 430 rpm.
2. The blend of step 1 was granulated by initiating the addition of water with plows and choppers set at approximately 430 and 1800 rpm, respectively. All of the water was added within approximately 4 minutes.
3. The granulation was continued for approximately 7 minutes.
4. The wet granulation was dried in a fluid bed dryer at an inlet temperature set point of 60° C. to achieve a target granulation LOD of 2%. A variation of ±0.5% moisture content was acceptable.
5. The dried granulation was passed through a Model “M” Fitzmill equipped with a #2A plate, set at a high speed (4500-4600 rpm), and impact set forward.
6. The granulation of step 5 was mixed in a V-blender for approximately 10 minutes at approximately 22 rpm.
7. About 100 g of the blend of step 6 was removed for use in step 8.
8. Magnesium stearate (MS) was added through a #20 screen, in approximately equal portions, to each side of the V-blender. After the MS addition, the blend of step 7 was added, in approximately equal portions, to each side of the V-blender and blended for approximately 3 minutes. The quantity of MS added was adjusted on a per tablet basis based on the quantity of granulation blended.
9. The lubricated granulation of step 8 was discharged into a double-bagged polyethylene bag with a desiccant bag between the bags.
10. The lubricated CE granulation was then compressed into 120 mg tablets using a ¼ inch round convex tooling with a Korsch XL100 compression machine. The tablets had a hardness range of 7.5-9.5 kp and thickness range of 0.14-0.16 inches.

TABLE 48
Composition of Conjugated Estrogens Internal Tablet Portion
with 10% HPMC K100M CR

| Description | Input/Tablet (mg) | % W/W |
|---|---|---|
| CE Desiccation with Lactose at 42.9 mg/g | 10.4895 | 8.74 |
| Lactose Monohydrate Spray Dried | 79.2105 | 66.01 |
| Avicel PH 101, NF | 18 | 15.00 |
| HPMC K100M Premium CR | 12 | 10.00 |
| Magnesium Stearate, NF | 0.3 | 0.25 |
| Purified Water, USP (A) | 30 | |

Note:
(A) Indicates removed during processing.

### Example 34A

### Preparation of the Tablet-In-Tablet Composition

Using the BZA granulation of Example 23 and the CE internal tablet of Example 34, a CE/BZA tablet-in-tablet was compressed using a Kilian RUD compression machine with an 11 mm round convex tooling. The target total tablet-in-tablet composition weight was 420 mg with 300 mg and 120 mg for the BZA external layer and CE internal tablet portion for the immediate release formulation. The fill weights of both sides (top and bottom) were adjusted in order to allow the CE internal tablet to position itself at the center of the finished tablet. Since the hardness measurement of the tablet-in-tablet composition was not consistent due to capping during the testing, which is common problem for tablet-in-tablet compositions, the compression force was based on the hardness of the tablet-in-tablet composition with the MPA external layer only. The targeted hardness of the MPA outer layer tablet alone had a range of 4.0-7.0 kp. Under this compression force, the tablet-in-tablet composition had a friability of zero percent.

### Example 34B

### Preparation of the Tablet-In-Tablet Composition

Using the BZA granulation of Example 24 and the CE core tablet of Example 34, a CE/BZA tablet-in-tablet composition was prepared by following the procedure of Example 34A.

### Example 34C

### Preparation of the Tablet-In-Tablet Composition

Using the BZA granulation of Example 25 and the CE core tablet of Example 34, a CE/BZA tablet-in-tablet composition was prepared by following the procedure of Example 34A.

### Example 34D

### Reparation of the Tablet-In-Tablet Composition

Using the BZA granulation of Example 26 and the CE core tablet of Example 34, a CE/BZA tablet-in-tablet composition was prepared by following the procedure of Example 34A.

### Example 34E

### Preparation of the Tablet-In-Tablet Composition

Using the BZA granulation of Example 27 and the CE core tablet of Example 34, a CE/BZA tablet-in-tablet composition was prepared by following the procedure of Example 34A.

### Example 34F

### Preparation of the Tablet-In-Tablet Composition

Using the BZA granulation of Example 28 and the CE core tablet of Example 34, a CE/BZA tablet-in-tablet composition was prepared by following the procedure of Example 34A.

### Example 34G

### Preparation of the Tablet-In-Tablet Composition

Using the BZA granulation of Example 29 and the CE core tablet of Example 34, a CE/BZA tablet-in-tablet composition was prepared by following the procedure of Example 34A.

### Example 34H

### Preparation of the Tablet-In-Tablet Composition

Using the BZA granulation of Example 30 and the CE core tablet of Example 34, a CE/BZA tablet-in-tablet composition was prepared by following the procedure of Example 34A.

### Example 341

### Preparation of the Tablet-In-Tablet Composition

Using the BZA granulation of Example 31 and the CE core tablet of Example 34, a CE/BZA tablet-in-tablet composition was prepared by following the procedure of Example 34A.

### Example 34J

### Preparation of the Tablet-In-Tablet Composition

Using the BZA granulation of Example 32 and the CE core tablet of Example 34, a CE/BZA tablet-in-tablet composition was prepared by following the procedure of Example 34A.

### Example 34-IR-1

### Preparation of the Tablet-In-Tablet Composition

Using the BZA granulation of Example 33 and the CE core tablet of Example 1, a CE/BZA tablet-in-tablet composition was compressed using a Kilian RUD compression machine with 11 mm round convex tooling. The target tablet-in-tablet composition weight was 520 mg with 400 mg for the BZA outer tablet layer for the immediate release formulation. The fill weights of both sides (top and bottom) were adjusted in order to allow the CE core tablet to position itself at the center of the finished tablet. Since the hardness measurement of the tablet-in-tablet composition was not consistent due to capping during the testing, which is common problem for tablet-in-tablet compositions, the compression force was based on the hardness of the tablet-in-tablet composition with the BZA outer tablet layer only. The targeted hardness of the BZA outer tablet layer alone had a range of 4.0-7.0 kp. Under this compression force, the tablet-in-tablet composition had a friability of zero percent.

### Example 34-IR-2

### Preparation of the Tablet-In-Tablet Composition

Using the BZA granulation of Example 33 and the CE core tablet of Example 2, a CE/BZA tablet-in-tablet composition was prepared by following the procedure of Example 341R-1.

### Example 34-IR-3

### Preparation of the Tablet-In-Tablet Composition

Using the BZA granulation of Example 33 and the CE core tablet of Example 3, a CE/BZA tablet-in-tablet composition was prepared by following the procedure of Example 341R-1.

### Example 35

### Characterization of the CE/BZA Tablet-In-Tablet Compositions

### Weight Variation

Weight variation of 100 tablet-in-tablet compositions was evaluated using the Mocon Automatic Balance Analysis tester for Examples 34A, 34B, and 34C.

Dissolution of BZA from Tablet-in-tablet Compositions

The dissolution of BZA for Examples 34A to 34J was determined using USP Apparatus 1 (basket), at 75 rpm in 900 mL of 10 mM acetate acid solution with 0.2% polysorbate 80 (Tween 80) at 37° C.±0.5° C. for a period of 60 minutes. Then the speed was changed to 250 rpm for data point at 80 minutes. A filtered sample of the dissolution medium was taken at specified time intervals. The release of the active was determined by reversed phase high performance liquid chromatography (HPLC).

### Results

### Weight Variation of MPA and CE of Tablet-in-tablets

Table 49 shows the results for the weight variation for Examples 34A-34C and Examples 34-IR-1 to 34-IR-3. From the data it can be seen that the compression process can produce a well-controlled tablet weight variation.

Dissolution Profiles of CE and BZA from Tablet-in-tablet Compositions

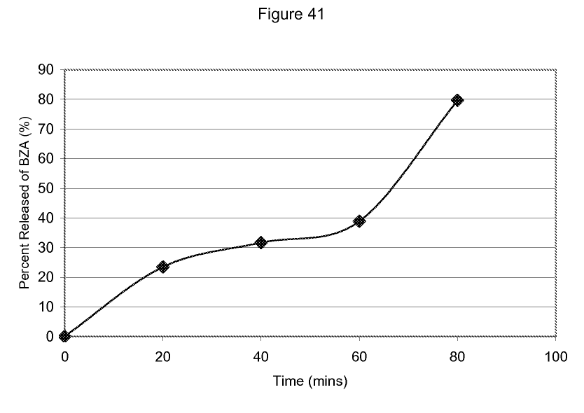

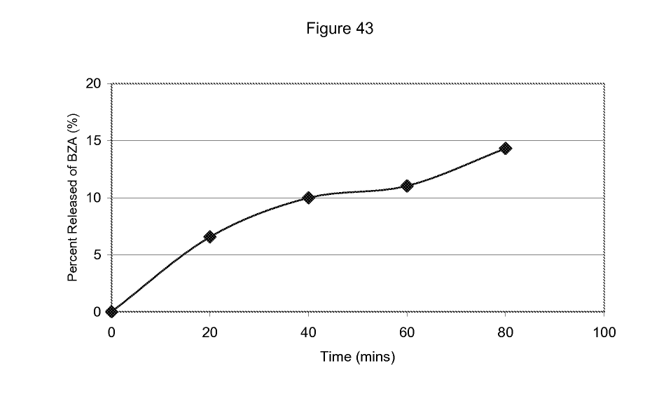

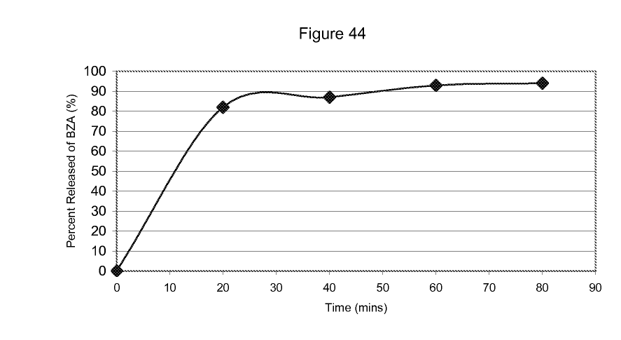

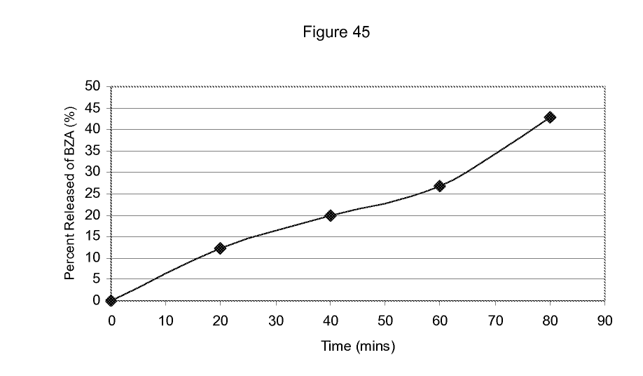

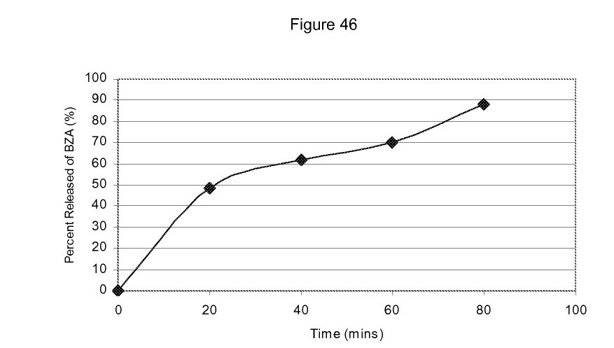

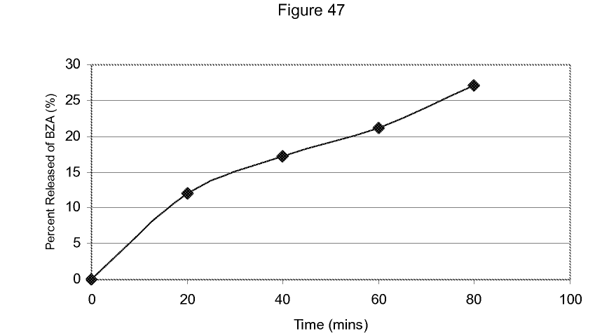

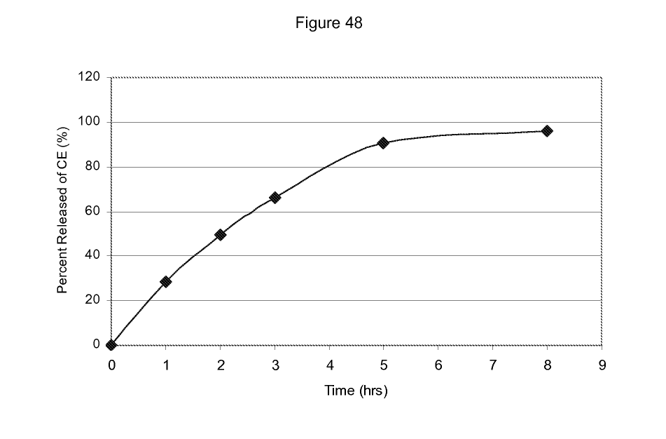

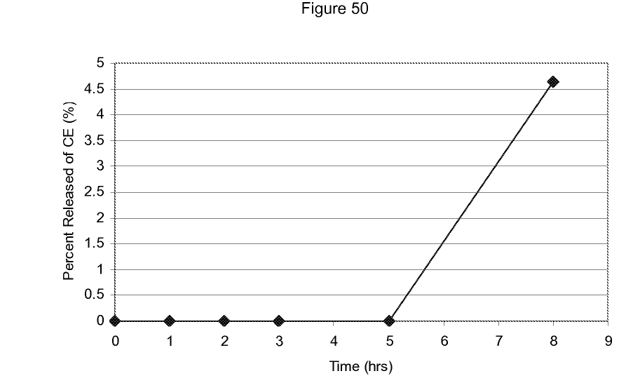

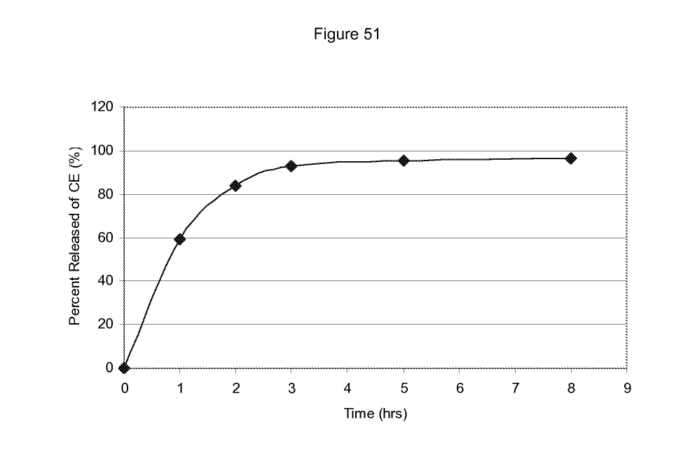

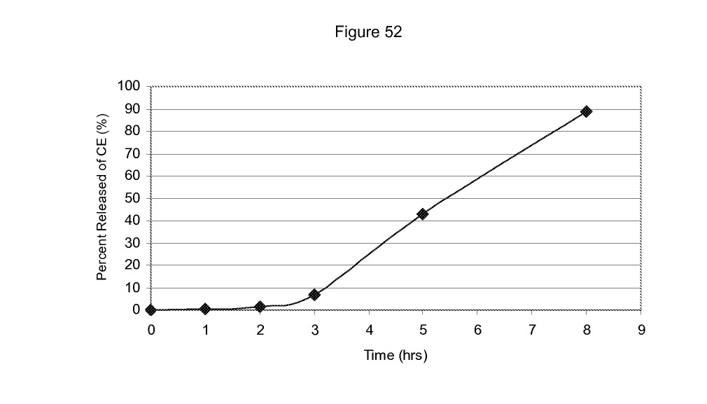

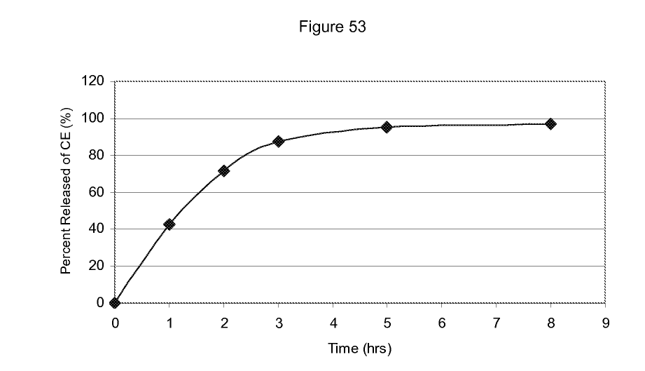

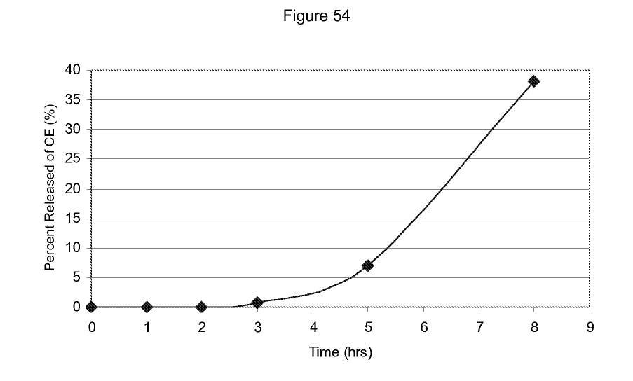

The dissolution profiles of BZA and CE from Examples 34A to 34J are listed in Tables 50 and 52 (BZA), Tables 51 and 53 (CE) and shown in FIGS. 27-29 and 41-47 (BZA) and FIGS. 30-32 and 48-54 (CE). From the results, it can be seen that a high level of polymer in the BZA layer will slow down the dissolution rates of both BZA and CE from the tablet-in-tablet composition.

From this study it can be concluded that the tablet-in-tablet composition and its related manufacturing procedures are robust to produce CE/BZA tablet-in-tablet compositions with excellent weight variation. A high level of polymer in the BZA outer tablet layer will slow down the dissolution rates of both BZA and CE from the tablet-in-tablet composition.

TABLE 49
Weight Variations of CE/BZA Tablet-in-tablets

| Batch # | Weight Variation (%) |
|---|---|
| Example 34A | 0.94 |
| Example 34B | 0.78 |
| Example 34C | 0.76 |
| Example 34-IR-1 | 1.82 |
| Example 34-IR-2 | 1.26 |
| Example 34-IR-3 | 1.08 |

TABLE 50
Dissolution of BZA from CE/BZA Tablet-in-tablets

| | Percent Dissolved (% ± sd, n = 2) | | |
|---|---|---|---|
| Time (minute) | Example 34A | Example 34B | Example 34C |
| 0 | 0 | 0 | 0 |
| 20 | 28 ± 2.8 | 11 ± 0.2 | 6 ± 0.5 |
| 40 | 41 ± 0.5 | 16 ± 2.9 | 9 ± 0.2 |
| 60 | 50 ± 1.3 | 20 ± 3.3 | 12 ± 0.4 |
| 80 | 84 ± 0.5 | 28 ± 2.4 | 15 ± 0.8 |

TABLE 51
Dissolution of CE from CE/BZA Tablet-in-tablets

| | Percent Dissolved (% ± sd, n = 6) | | |
|---|---|---|---|
| Time (hr) | Example 34A | Example 34B | Example 34C |
| 0 | 0 | 0 | 0 |
| 1 | 13.04 ± 10.6 | 0.32 ± 0.8 | 0 ± 0 |
| 2 | 31.24 ± 16.2 | 1.57 ± 2.4 | 0 ± 0 |
| 3 | 51.29 ± 14.4 | 3.95 ± 3.7 | 0 ± 0 |
| 5 | 81.22 ± 5.8 | 16.32 ± 5.2 | 0.57 ± 1.4 |
| 8 | 93.8 ± 1.5 | 45.43 ± 5.5 | 8.22 ± 6.0 |

TABLE 52
Dissolution of BZA from CE/BZA Tablet-in-Table Compositions

| | Percent Dissolved (% ± sd, n = 3) | | | | | | |
|---|---|---|---|---|---|---|---|
| Time | Example | Example | Example | Example | Example | Example | Example |
| (min) | 34D | 34E | 34F | 34G | 34H | 34I | 34J |
| 0 | 0 | 0 | 0 | 0 | 0 | 0 | 0 |
| 20 | 23 ± 1.7 | 11 ± 1.1 | 7 ± 1.0 | 82 ± 3.4 | 12 ± 2.9 | 49 ± 4.1 | 12 ± 1.6 |
| 40 | 32 ± 2.8 | 15 ± 1.7 | 10 ± 1.4 | 87 ± 4.4 | 20 ± 4.2 | 62 ± 6.4 | 17 ± 2.6 |
| 60 | 39 ± 2.3 | 18 ± 1.6 | 11 ± 1.6 | 93 ± 3.6 | 27 ± 3.4 | 70 ± 9.4 | 21 ± 3.1 |
| 80 | 80 ± 10.2 | 24 ± 2.4 | 14 ± 1.8 | 94 ± 3.0 | 43 ± 6.9 | 88 ± 3.4 | 27 ± 1.2 |

TABLE 53
Dissolution of CE from CE/BZA Tablet-in-Tablet Compositions

| | Percent Dissolved (% ± sd, n = 6) | | | | | | |
|---|---|---|---|---|---|---|---|
| Time | Example | Example | Example | Example | Example | Example | Example |
| (hr) | 34D | 34E | 34F | 34G | 34H | 34I | 34J |
| 0 | 0 | 0 | 0 | 0 | 0 | 0 | 0 |
| 1 | 29 ± 3.0 | 4 ± 5.9 | 0 ± 0 | 59 ± 10.5 | 0.3 ± 0.8 | 43 ± 7.6 | 0 ± 0 |
| 2 | 50 ± 5.3 | 10 ± 11.5 | 0 ± 0 | 84 ± 11.9 | 1.4 ± 2.6 | 72 ± 10.8 | 0 ± 0 |
| 3 | 66 ± 8.9 | 16 ± 14.1 | 0 ± 0 | 93 ± 9.9 | 7 ± 5.3 | 87 ± 10.3 | 0.7 ± 1.2 |
| 5 | 90 ± 5.7 | 29 ± 16.0 | 0 ± 0 | 95 ± 7.7 | 43 ± 4.4 | 95 ± 6.0 | 7 ± 5.6 |
| 8 | 96 ± 2.5 | 53 ± 13.2 | 5 ± 2.9 | 97 ± 5.5 | 89 ± 2.2 | 97 ± 3.8 | 38 ± 11.9 |

Various modifications of the invention, in addition to those described herein, will be apparent to those skill in the art from the foregoing description. Such modifications are also intended to fall into the scope of the claims. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A tablet-in-tablet composition comprising:
a) a core tablet comprising:
one or more estrogens;
a core filler/diluent component comprising from about 30% to about 85% by weight of said core tablet;
a core filler/binder component comprising from about 1% to about 30% by weight of said core tablet;
a core hydrophilic gel-forming polymer component comprising from about 1% to about 40% by weight of said core tablet; and
optionally, a core lubricant component comprising from about 0.01% to about 2% by weight of said core tablet; and
b) a compressed outer tablet layer comprising:
one or more therapeutic agents selected from the group consisting of selective estrogen receptor modulators and progestational agents;
an outer layer filler/diluent component comprising from about 10% to about 80% by weight of said compressed outer tablet layer;
an outer layer filler/binder component comprising from about 1% to about 60% by weight of said compressed outer tablet layer;
an outer layer hydrophilic gel-forming polymer component comprising from about 1% to about 70% by weight of said compressed outer tablet layer;
optionally, an antioxidant component comprising from about 0.01% to about 4% by weight of said compressed outer tablet layer; and
optionally, an outer layer lubricant component comprising from about 0.01% to about 2% by weight of said compressed outer tablet layer.

2. The tablet-in-tablet composition of claim 1 wherein:
said core tablet comprises from about 10% to about 50% by weight of said composition; and
said compressed outer tablet layer comprises from about 50% to about 90% by weight of said composition.

3. The tablet-in-tablet composition of claim 1 or claim 2 wherein said compressed outer tablet layer has a hardness from about 2 kp to about 7 kp.

4. The tablet-in-tablet composition of any one of claims 1 to 3 wherein said compressed outer tablet layer does not comprise a surfactant or wetting agent.

5. The tablet-in-tablet composition of claim 1 wherein:
said core tablet comprises from about 10% to about 50% by weight of said composition;
said compressed outer tablet layer comprises from about 50% to about 90% by weight of said composition;
said compressed outer tablet layer has a hardness from about 2 kp to about 7 kp; and
said compressed outer tablet layer does not comprise a surfactant or wetting agent.

6. The tablet-in-tablet composition of any one of claims 1 to 5 wherein said core tablet comprises at least one conjugated estrogen.

7. The tablet-in-tablet composition of any one of claims 1 to 6 wherein said compressed outer tablet layer comprises bazedoxifene, or pharmaceutically acceptable salt thereof.

8. The tablet-in-tablet composition of claim 7 wherein said compressed outer tablet layer comprises bazedoxifene acetate.

9. The tablet-in-tablet composition of any one of claims 1 to 6 wherein said compressed outer tablet layer comprises medroxyprogesterone acetate.

10. The tablet-in-tablet composition of claim 1 wherein:
said core tablet comprises at least one conjugated estrogen; and
said compressed outer tablet layer comprises medroxyprogestrone acetate or bazedoxifene acetate.

11. The tablet-in-tablet composition of any one of claims 1 to 10 wherein:
said core filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said core filler/binder component comprises one or more of microcrystalline cellulose, polyvinylpyrrolidone, copovidone, polyvinylalcohol, starch, gelatin, gum arabic, gum acacia, and gum tragacanth;
said core hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said outer layer filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said outer layer filler/binder component comprises one or more of microcrystalline cellulose, polyvinylpyrrolidone, copovidone, polyvinylalcohol, starch, gelatin, gum arabic, gum acacia, and gum tragacanth;
said outer layer hydrophilic gel-forming polymer comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional antioxidant component, if present, comprises one or more of ascorbic acid, sodium ascorbate, ascorbyl palmitate, vitamin E, vitamin E acetate, butylated hydroxytoluene, and butylated hydroxyanisole.

12. The tablet-in-tablet composition of claim 1 wherein:
said core filler/diluent component comprises one or more of lactose and lactose monohydrate;
said core filler/binder component comprises microcrystalline cellulose;
said core hydrophilic gel-forming polymer component comprises hydroxypropylmethylcellulose;
said optional core lubricant component, if present, comprises magnesium stearate;
said core filler/diluent component comprises one or more of lactose and lactose monohydrate;
said outer layer filler/binder component comprises microcrystalline cellulose;
said outer layer hydrophilic gel-forming polymer comprises hydroxypropylmethylcellulose;
said optional outer layer lubricant component, if present, comprises magnesium stearate;
said optional antioxidant component, if present, comprises one or more of ascorbic acid and vitamin E acetate;
said core tablet comprises at least one conjugated estrogen; and
said compressed outer tablet layer comprises medroxyprogesterone acetate or bazedoxifene acetate.

13. The tablet-in-tablet composition of any one of claims 1 to 12 wherein:
said core filler/diluent component comprises from about 50% to about 85% by weight of said core tablet;
said core filler/binder component comprises from about 10% to about 20% by weight of said core tablet;
said core hydrophilic gel-forming polymer component comprises from about 5% to about 15% by weight of said core tablet; and
said outer layer hydrophilic gel-forming polymer component comprises from about 1% to about 8% by weight of said compressed outer tablet layer.

14. The tablet-in-tablet composition of any one of claims 1 to 12 wherein:
said core filler/diluent component comprises from about 50% to about 85% by weight of said core tablet;
said core filler/binder component comprises from about 10% to about 20% by weight of said core tablet;
said core hydrophilic gel-forming polymer component comprises from about 5% to about 15% by weight of said core tablet; and
said outer layer hydrophilic gel-forming polymer component comprises from about 8% to about 15% by weight of said compressed outer tablet layer.

15. The tablet-in-tablet composition of any one of claims 1 to 12 wherein:
said core filler/diluent component comprises from about 50% to about 85% by weight said core tablet;
said core filler/binder component comprises from about 10% to about 20% by weight of said core tablet;
said core hydrophilic gel-forming polymer component comprises from about 5% to about 15% by weight of said core tablet; and
said outer layer hydrophilic gel-forming polymer component comprises from about 15% to about 30% by weight of said compressed outer tablet layer.

16. The tablet-in-tablet composition of any one of claims 1 to 12 wherein:
said core filler/diluent component comprises from about 50% to about 85% by weight of said core tablet;
said core filler/binder component comprises from about 10% to about 20% by weight of said core tablet said;
said core hydrophilic gel-forming polymer component comprises from about 5% to about 15% by weight of said core tablet; and
said outer layer hydrophilic gel-forming polymer component comprises from about 30% to about 50% by weight of said compressed outer tablet layer.

17. The tablet-in-tablet composition of any one of claims 1 to 12 wherein:
said core filler/diluent component comprises from about 50% to about 85% by weight of said core tablet;
said core filler/binder component comprises from about 10% to about 20% by weight of said core tablet;
said core hydrophilic gel-forming polymer component comprises from about 15% to about 25% by weight of said core tablet; and
said outer layer hydrophilic gel-forming polymer component comprises from about 1% to about 8% by weight of said compressed outer tablet layer.

18. The tablet-in-tablet composition of any one of claims 1 to 12 wherein:
said core filler/diluent component comprises from about 50% to about 85% by weight of said core tablet;
said core filler/binder component comprises from about 10% to about 20% by weight of said core tablet;
said core hydrophilic gel-forming polymer component comprises from about 15% to about 25% by weight of said core tablet; and
said outer layer hydrophilic gel-forming polymer component comprises from about 8% to about 15% by weight of said compressed outer tablet layer.

19. The tablet-in-tablet composition of any one of claims 1 to 12 wherein:
said core filler/diluent component comprises from about 50% to about 85% by weight of said core tablet;
said core filler/binder component comprises from about 10% to about 20% by weight of said core tablet;
said core hydrophilic gel-forming polymer component comprises from about 15% to about 25% by weight of said core tablet; and
said outer layer hydrophilic gel-forming polymer component comprises from about 15% to about 30% by weight of said compressed outer tablet layer.

20. The tablet-in-tablet composition of any one of claims 1 to 12 wherein:
said core filler/diluent component comprises from about 50% to about 85% by weight of said core tablet;
said core filler/binder component comprises from about 10% to about 20% by weight of said core tablet;
said core hydrophilic gel-forming polymer component comprises from about 15% to about 25% by weight of said core tablet; and
said outer layer hydrophilic gel-forming polymer component comprises from about 30% to about 50% by weight of said compressed outer tablet layer.

21. The tablet-in-tablet composition of any one of claims 1 to 12 wherein:
said core filler/diluent component comprises from about 40% to about 75% by weight of said core tablet;
said core filler/binder component comprises from about 10% to about 20% by weight of said core tablet;
said core hydrophilic gel-forming polymer component comprises from about 25% to about 35% by weight of said core tablet; and
said outer layer hydrophilic gel-forming polymer component comprises from about 1% to about 8% by weight of said compressed outer tablet layer.

22. The tablet-in-tablet composition of any one of claims 1 to 12 wherein:
said core filler/diluent component comprises from about 40% to about 75% by weight of said core tablet;
said core filler/binder component comprises from about 10% to about 20% by weight of said core tablet;
said core hydrophilic gel-forming polymer component comprises from about 25% to about 35% by weight of said core tablet; and
said outer layer hydrophilic gel-forming polymer component comprises from about 8% to about 15% by weight of said compressed outer tablet layer.

23. The tablet-in-tablet composition of any one of claims 1 to 12 wherein:
said core filler/diluent component comprises from about 40% to about 75% by weight of said core tablet;
said core filler/binder component comprises from about 10% to about 20% by weight of said core tablet;
said core hydrophilic gel-forming polymer component comprises from about 25% to about 35% by weight of said core tablet; and
said outer layer hydrophilic gel-forming polymer component comprises from about 15% to about 30% by weight of said compressed outer tablet layer.

24. The tablet-in-tablet composition of any one of claims 1 to 12 wherein:
said core filler/diluent component comprises from about 40% to about 75% by weight of said core tablet;
said core filler/binder component comprises from about 10% to about 20% by weight of said core tablet;
said core hydrophilic gel-forming polymer component comprises from about 25% to about 35% by weight of said core tablet; and
said outer layer hydrophilic gel-forming polymer component comprises from about 30% to about 50% by weight of said compressed outer tablet layer.

25. A tablet-in-tablet composition selected from a plurality of compositions according to any one of claims 1 to 24, wherein the plurality has a mean dissolution profile wherein:
the mean of % of the estrogen released per composition after 1, 2, 3, 4, and 5 hours under estrogen dissolution conditions is substantially equal to the sum of b₁*X₁,b₂X₂, b₃*X₃, b₁₂*X₁*X₂, b₁₃*X₁*X₃, and b₂₃*X₂*X₃; and
the mean of % of the therapeutic agent per composition released after 0.25, 0.5, 1, 2, and 6 hours under type I therapeutic agent dissolution conditions is substantially equal to the sum of a₁*X₁,b₂X₂, a₃*X₃, a₁₂*X₁*X₂, a₁₃*X₁*X₃, and a₂₃*X₂*X₃;
X₁ is the % by weight of said outer layer hydrophilic gel-forming polymer component in said compressed outer tablet layer;
X₂ is the % by weight of said outer layer filler/diluent component in said compressed outer tablet layer;
X₃ is the % by weight of said outer layer filler/binder component in said compressed outer tablet layer;
b₁ at 1 hour is 157.4;
b₁ at 2 hours is 193.09;
b₁ at 3 hours is 184.1;
b₁ at 4 hours is 146.45;
b₁ at 5 hours is 100.25;
b₂ at 1 hour is 54.47;
b₂ at 2 hours is 80.09;
b₂ at 3 hours is 93.71;
b₂ at 4 hours is 101.05;
b₂ at 5 hours is 104.11;
b₃ at 1 hour is 46.75;
b₃ at 2 hours is 69.86;
b₃ at 3 hours is 84.19;
b₃ at 4 hours is 92.12;
b₃ at 5 hours is 95.89;
b₁₂ at 1 hour is −437.12;
b₁₂ at 2 hours is −557.91;
b₁₂ at 3 hours is −561.48;
b₁₂ at 4 hours is −489.08;
b₁₂ at 5 hours is −383.44;
b₁₃ at 1 hour is −414.17;
b₁₃ at 2 hours is −542.65;
b₁₃ at 3 hours is −569.13;
b₁₃ at 4 hours is −518.63;
b₁₃ at 5 hours is −441.05;
b₂₃ at 1 hour is 76.74;
b₂₃ at 2 hours is 79.7;
b₂₃ at 3 hours is 65.43;
b₂₃ at 4 hours is 43.23;
b₂₃ at 5 hours is 29.91;
a₁ at 0.25 hour is 217.8;
a₁ at 0.5 hour is 218.36;
a₁ at 1 hour is 188.75;
a₁ at 2 hours is 121.23;
a₁ at 6 hours is −21.48;
a₂ at 0.25 hour is 87.91;
a₂ at 0.5 hour is 93.12;
a₂ at 1 hour is 96.98;
a₂ at 2 hours is 100.52;
a₂ at 6 hours is 100.91;
a₃ at 0.25 hour is 58.83;
a₃ at 0.5 hour is 75.08;
a₃ at 1 hour is 86.32;
a₃ at 2 hours is 92.04;
a₃ at 6 hours is 99.99;
a₁₂ at 0.25 hour is −616.98;
a₁₂ at 0.5 hour is −617.39;
a₁₂ at 1 hour is −545.68;
a₁₂ at 2 hours is −377.76;
a₁₂ at 6 hours is 69.72;
a₁₃ at 0.25 hour is −536.63;
a₁₃ at 0.5 hour is −576.95;
a₁₃ at 1 hour is −540.35;
a₁₃ at 2 hours is −397.91;
a₁₃ at 6 hours is 12.22;
a₂₃ at 0.25 hour is 30.77;
a₂₃ at 0.5 hour is 31.94;
a₂₃ at 1 hour is 32.68;
a₂₃ at 2 hours is 32.91; and
a₂₃ at 6 hours is 9.65.

26. The tablet-in-tablet composition of claim 1 wherein:
said core tablet comprises at least one conjugated estrogen;
said compressed outer tablet layer comprises bazedoxifene acetate;
said dissolution profile of said estrogen from said tablet under estrogen dissolution conditions is substantially as shown in any one of FIGS. 30 to 32 or 48 to 54; and
said dissolution profile of said therapeutic agent from said tablet under type II therapeutic agent dissolution conditions is substantially as shown in any one of FIGS. 27 to 29 or 41 to 47.

27. The tablet-in-tablet composition of claim 1 wherein:
said core tablet comprises at least one conjugated estrogen;
said compressed outer tablet layer comprises medroxyprogesterone acetate;
said dissolution profile of said estrogen from said tablet under estrogen dissolution conditions is substantially as shown in any one of FIGS. 4-6, FIG. 33 (Example 9), FIG. 34 (Example 13), FIG. 35 (Example 15), FIG. 35 (Example 16), FIG. 35 (Example 18) or FIG. 36 (Example 20); and
said dissolution profile of said therapeutic agent from said tablet under type I therapeutic agent dissolution conditions is substantially as shown in any one of FIGS. 1-3, FIG. 37 (Example 9), FIG. 38 (Example 13), FIG. 39 (Example 15), FIG. 39 (Example 16), FIG. 39 (Example 18) or FIG. 40 (Example 20).

28. A tablet-in-tablet composition selected from a plurality of tablet-in-tablet compositions according to any one of claims 1 to 27, wherein said plurality has a content uniformity for said therapeutic agent about equal to or less than 3.5%.

29. A tablet-in-tablet composition selected from a plurality of tablet-in-tablet compositions according to any one of claims 1 to 27, wherein said plurality has a content uniformity for said therapeutic agent about equal to or less than 2.5%.

30. A tablet-in-tablet composition selected from a plurality of tablet-in-tablet compositions according to any one of claims 1 to 27, wherein said plurality has a weight variation of about equal to or less than 2%.

31. A tablet-in-tablet composition selected from a plurality of tablet-in-tablet compositions according to any one of claims 1 to 27, wherein said plurality has a weight variation of about equal to or less than 1.5%.

32. A tablet-in-tablet composition comprising:
a) a core tablet comprising:
one or more estrogens;
a core filler/diluent component comprising from about 30% to about 85% by weight by weight of said core tablet;
a core filler/binder component comprising from about 1% to about 30% by weight of said core tablet;
a core hydrophilic gel-forming polymer component comprising from about 1% to about 40% by weight of said core tablet; and
optionally, a core lubricant component comprising from about 0.01% to about 2% by weight of said core tablet; and
b) a compressed outer tablet layer comprising:
one or more therapeutic agents selected from the group consisting of selective estrogen receptor modulators and progestational agents;
a pharmaceutically acceptable carrier component comprising from about 60% to about 99.9% by weight of said compressed outer tablet layer, wherein said pharmaceutically acceptable carrier component optionally comprises one or more of an outer layer filler/diluent component, an outer layer filler/binder component, and an outer layer hydrophilic gel-forming polymer component;
optionally, an outer layer lubricant component comprising from about 0.01% to about 2% by weight of said compressed outer tablet layer; and
optionally, an antioxidant component comprising from about 0.01% to about 4% by weight of said compressed outer tablet layer.

33. The tablet-in-tablet composition of claim 32 wherein:
said core tablet comprises from about 10% to about 50% by weight of said composition; and
said compressed outer tablet layer comprises from about 50% to about 90% by weight of said composition.

34. The tablet-in-tablet composition of claim 32 or claim 33 wherein said compressed outer tablet layer has a hardness from about 2 kp to about 7 kp.

35. The tablet-in-tablet composition of any one of claims 32 to 34 wherein said compressed outer tablet layer does not comprise a surfactant or wetting agent.

36. The tablet-in-tablet composition of claim 32 wherein:
said core tablet comprises from about 10% to about 50% by weight of said composition;
said compressed outer tablet layer comprises from about 50% to about 90% by weight of said composition;
said compressed outer tablet layer has a hardness from about 2 kp to about 7 kp; and
said compressed outer tablet layer does not comprise a surfactant or wetting agent.

37. The tablet-in-tablet composition of any one of claims 32 to 36 wherein said pharmaceutically acceptable carrier component comprises an outer layer filler/diluent component.

38. The tablet-in-tablet composition of any one of claims 32 to 37 wherein said pharmaceutically acceptable carrier component comprises an outer layer filler/binder component.

39. The tablet-in-tablet composition of any one of claims 32 to 38 wherein said pharmaceutically acceptable carrier component comprises an outer layer hydrophilic gel-forming polymer component.

40. The tablet-in-tablet composition of any one of claims 32 to 36 wherein said pharmaceutically acceptable carrier component comprises:
from about 30% to about 99.9% by weight of an outer layer filler/diluent component; and
from about 1% to about 70% by weight of an outer layer filler/binder component.

41. The tablet-in-tablet composition of any one of claims 32 to 36 wherein said pharmaceutically acceptable carrier component comprises:
from about 30% to about 99.9% by weight of an outer layer filler/diluent component; and
from about 1% to about 70% by weight of an outer layer hydrophilic gel-forming polymer component.

42. The tablet-in-tablet composition of any one of claims 32 to 36 wherein said pharmaceutically acceptable carrier component comprises:
from about 30% to about 99.9% by weight of an outer layer filler/binder component; and
from about 1% to about 70% by weight of an outer layer hydrophilic gel-forming polymer component.

43. The tablet-in-tablet composition of any one of claims 32 to 42 wherein:
said core filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said core filler/binder component comprises one or more of microcrystalline cellulose, polyvinylpyrrolidone, copovidone, polyvinylalcohol, starch, gelatin, gum arabic, gum acacia, and gum tragacanth;
said core hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said pharmaceutically acceptable carrier component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, a metal carbonate, polyvinylpyrrolidone, copovidone, polyvinylalcohol, gelatin, gum arabic, gum acacia, gum tragacanth, hydroxypropylmethylcellulose, polyethylene oxide, hydroxypropylcellulose, hydroxyethylcellulose, methylcellulose, xanthan gum, and guar gum;
said optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional antioxidant component, if present, comprises one or more of ascorbic acid, sodium ascorbate, ascorbyl palmitate, vitamin E, vitamin E acetate, butylated hydroxytoluene, and butylated hydroxyanisole;
said core tablet comprises at least one conjugated estrogen; and
said compressed outer tablet layer comprises medroxyprogesterone acetate or bazedoxifene acetate.

44. The tablet-in-tablet composition of any one of claims 32 to 42 wherein:
said core filler/diluent component comprises one or more of lactose and lactose monohydrate;
said core filler/binder component comprises microcrystalline cellulose;
said core hydrophilic gel-forming polymer component comprises hydroxypropylmethylcellulose;
said optional core lubricant component, if present, comprises magnesium stearate;
said pharmaceutically acceptable carrier component comprises one or more of lactose, lactose monohydrate, microcrystalline cellulose, and hydroxypropylmethylcellulose;
said optional outer layer lubricant component, if present, comprises magnesium stearate;
said optional antioxidant component, if present, comprises one or more of ascorbic acid and vitamin E acetate;
said core tablet comprises at least one conjugated estrogen; and
said compressed outer tablet layer comprises medroxyprogesterone acetate or bazedoxifene acetate.

45. A tablet-in-tablet composition selected from a plurality of compositions according to any one of claims 32 to 44, wherein the plurality has a mean dissolution profile wherein:
the mean of % of the estrogen released per composition after 1, 2, 3, 4, and 5 hours under estrogen dissolution conditions is substantially equal to the sum of b₁*X₁,b₂X₂, b₃*X₃, b₁₂*X₁*X₂, b₁₃*X₁*X₃, and b₂₃*X₂*X₃;
the mean of % of the therapeutic agent per composition released after 0.25, 0.5, 1, 2, and 6 hours under type I therapeutic agent dissolution conditions is substantially equal to the sum of a₁*X₁,b₂X₂, a₃*X₃, a₁₂*X₁*X₂, a₁₃*X₁*X₃, and a₂₃*X₂*X₃;
X₁ is the % by weight of said optional outer layer hydrophilic gel-forming polymer component, if present, in said compressed outer tablet layer;
X₂ is the % by weight of said optional outer layer filler/diluent component, if present, in said compressed outer tablet layer; and
X₃ is the % by weight of said optional outer layer filler/binder component, if present, in said compressed outer tablet layer;
wherein:
b₁ at 1 hour is 157.4;
b₁ at 2 hours is 193.09;
b₁ at 3 hours is 184.1;
b₁ at 4 hours is 146.45;
b₁ at 5 hours is 100.25;
b₂ at 1 hour is 54.47;
b₂ at 2 hours is 80.09;
b₂ at 3 hours is 93.71;
b₂ at 4 hours is 101.05;
b₂ at 5 hours is 104.11;
b₃ at 1 hour is 46.75;
b₃ at 2 hours is 69.86;
b₃ at 3 hours is 84.19;
b₃ at 4 hours is 92.12;
b₃ at 5 hours is 95.89;
b₁₂ at 1 hour is −437.12;
b₁₂ at 2 hours is −557.91;
b₁₂ at 3 hours is −561.48;
b₁₂ at 4 hours is −489.08;
b₁₂ at 5 hours is −383.44;
b₁₃ at 1 hour is −414.17;
b₁₃ at 2 hours is −542.65;
b₁₃ at 3 hours is −569.13;
b₁₃ at 4 hours is −518.63;
b₁₃ at 5 hours is −441.05;
b₂₃ at 1 hour is 76.74;
b₂₃ at 2 hours is 79.7;
b₂₃ at 3 hours is 65.43;
b₂₃ at 4 hours is 43.23;
b₂₃ at 5 hours is 29.91;
a₁ at 0.25 hour is 217.8;
a₁ at 0.5 hour is 218.36;
a₁ at 1 hour is 188.75;
a₁ at 2 hours is 121.23;
a₁ at 6 hours is −21.48;
a₂ at 0.25 hour is 87.91;
a₂ at 0.5 hour is 93.12;
a₂ at 1 hour is 96.98;
a₂ at 2 hours is 100.52;
a₂ at 6 hours is 100.91;
a₃ at 0.25 hour is 58.83;
a₃ at 0.5 hour is 75.08;
a₃ at 1 hour is 86.32;
a₃ at 2 hours is 92.04;
a₃ at 6 hours is 99.99;
a₁₂ at 0.25 hour is −616.98;
a₁₂ at 0.5 hour is −617.39;
a₁₂ at 1 hour is −545.68;
a₁₂ at 2 hours is −377.76;
a₁₂ at 6 hours is 69.72;
a₁₃ at 0.25 hour is −536.63;
a₁₃ at 0.5 hour is −576.95;
a₁₃ at 1 hour is −540.35;
a₁₃ at 2 hours is −397.91;
a₁₃ at 6 hours is 12.22;
a₂₃ at 0.25 hour is 30.77;
a₂₃ at 0.5 hour is 31.94;
a₂₃ at 1 hour is 32.68;
a₂₃ at 2 hours is 32.91; and
a₂₃ at 6 hours is 9.65.

46. The tablet-in-tablet composition of claim 32 wherein:
said core tablet comprises at least one conjugated estrogen;
said compressed outer tablet layer comprises medroxyprogesterone acetate;
said dissolution profile of said estrogen from said tablet under estrogen dissolution conditions is substantially as shown in any one of FIG. 33 (Example 8), FIG. 33 (Example 10), FIG. 33 (Example 11), FIG. 34 (Example 12), FIG. 34 (Example 14), FIG. 35 (Example 17), FIG. 36 (Example 19) or FIG. 36 (Example 21); and
said dissolution profile of said therapeutic agent from said tablet under type I therapeutic agent dissolution conditions is substantially as shown in any one of FIG. 37 (Example 8), FIG. 37 (Example 10), FIG. 38 (Example 11), FIG. 38 (Example 12), FIG. 38 (Example 14), FIG. 39 (Example 17), FIG. 40 (Example 19) or FIG. 40 (Example 21).

47. A tablet-in-tablet composition selected from a plurality of tablet-in-tablet compositions according to claim 32, wherein said plurality has a content uniformity for said therapeutic agent about equal to or less than 3.5%.

48. A tablet-in-tablet composition selected from a plurality of tablet-in-tablet compositions according to claim 32, wherein said plurality has a content uniformity for said therapeutic agent about equal to or less than 2.5%.

49. A tablet-in-tablet composition selected from a plurality of tablet-in-tablet compositions according to claim 32, wherein said plurality has a weight variation of about equal to or less than 2%.

50. A tablet-in-tablet composition selected from a plurality of tablet-in-tablet compositions according to of claim 32, wherein said plurality has a weight variation of about equal to or less than 1.5%.

51. A tablet-in-tablet composition comprising:
a) a core tablet comprising:
one or more estrogens;
a core filler/diluent component comprising from about 30% to about 85% by weight by weight of said core tablet;
a core filler/binder component comprising from about 1% to about 30% by weight of said core tablet;
a core hydrophilic gel-forming polymer component comprising from about 1% to about 40% by weight of said core tablet; and
optionally, a core lubricant component comprising from about 0.01% 10 to about 2% by weight of said core tablet; and
b) a compressed outer tablet layer comprising:
one or more therapeutic agents selected from the group consisting of selective estrogen receptor modulators and progestational agents;
an outer layer filler/diluent component comprising from about 25% to about 65% by weight of said compressed outer tablet layer;
an outer layer filler/binder component comprising from about 20% to about 50% by weight of said compressed outer tablet layer;
a disintegrant component comprising from about 2% to about 15% by weight of said compressed outer tablet layer;
optionally, an outer layer wetting agent component comprising from about 0.01% to about 4% of said compressed outer tablet layer;
optionally, an outer layer lubricant component comprising from about 0.01% to about 2% by weight of said compressed outer tablet layer; and
optionally, an antioxidant component comprising from about 0.01% to about 4% by weight of said compressed outer tablet layer.

52. The tablet-in-tablet composition of claim 51 wherein:
said core filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said core filler/binder component comprises one or more of microcrystalline cellulose, polyvinylpyrrolidone, copovidone, polyvinylalcohol, starch, gelatin, gum arabic, gum acacia, and gum tragacanth;
said core hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional core lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said outer layer filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said outer layer filler/binder component comprises one or more of silicified microcrystalline cellulose, microcrystalline cellulose, polyvinylpyrrolidone, copovidone, polyvinylalcohol, starch, gelatin, gum arabic, gum acacia, and gum tragacanth;
said outer layer disintegrant component comprises one or more of croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional outer layer wetting agent component, if present, comprises one or more of a polyethylene glycol-polypropylene glycol copolymer, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, polyethoxylated fatty acid esters, and polyglycolized glycerides;
said optional outer layer lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional antioxidant component, if present, comprises one or more of ascorbic acid, sodium ascorbate, ascorbyl palmitate, vitamin E, vitamin E acetate, butylated hydroxytoluene, and butylated hydroxyanisole;
said core tablet comprises at least one conjugated estrogen; and
said compressed outer tablet layer comprises medroxyprogesterone acetate or bazedoxifene acetate.

53. The tablet-in-tablet composition of claim 51 wherein:
said core filler/diluent component comprises one or more of lactose and lactose monohydrate;
said core filler/binder component comprises microcrystalline cellulose;
said core hydrophilic gel-forming polymer component comprises hydroxypropylmethylcellulose;
said optional core lubricant component, if present, comprises magnesium stearate;
said outer layer filler/diluent component comprises lactose monohydrate;
said outer layer filler/binder component comprises microcrystalline cellulose;
said outer layer disintegrant component comprises one or more of pregelatinized starch and sodium starch glycolate;
said optional outer layer wetting agent component, if present, comprises a polyethylene glycol-polypropylene glycol copolymer;
said optional outer layer lubricant component, if present, comprises magnesium stearate;
said optional antioxidant component, if present, comprises one or more of ascorbic acid and vitamin E acetate;
said core tablet comprises at least one conjugated estrogen; and
said compressed outer tablet layer comprises medroxyprogesterone acetate or bazedoxifene acetate.

54. A tablet-in-tablet composition selected from a plurality of tablet-in-tablet compositions according to any one of claims 51 to 53, wherein said plurality has a content uniformity for said therapeutic agent about equal to or less than 3.5%.

55. A tablet-in-tablet composition selected from a plurality of tablet-in-tablet compositions according to any one of claims 51 to 53, wherein said plurality has a content uniformity for said therapeutic agent about equal to or less than 2.5%.

56. A tablet-in-tablet composition selected from a plurality of tablet-in-tablet compositions according to any one of claims 51 to 53, wherein said plurality has a weight variation of about equal to or less than 2%.

57. A tablet-in-tablet composition selected from a plurality of tablet-in-tablet compositions according to of any one of claims 51 to 53, wherein said plurality has a weight variation of about equal to or less than 1.5%.

58. A process for producing a tablet-in-tablet composition comprising:
compressing a first solid mixture to form a core tablet; and
compressing a second solid mixture onto said core tablet to form a compressed outer tablet layer;
wherein:
(a) said first solid mixture comprises:
one or more estrogens;
a first solid mixture filler/diluent component comprising from about 30% to about 85% by weight of said first solid mixture;
a first solid mixture filler/binder component comprising from about 1% to about 30% by weight of said first solid mixture;
a first solid mixture hydrophilic gel-forming polymer component comprising from about 1% to about 40% by weight of said first solid mixture; and
optionally, a first solid mixture lubricant component comprising from about 0.01% to about 2% by weight of said first solid mixture; and
(b) said second solid mixture comprises:
one or more therapeutic agents selected from the group consisting of selective estrogen receptor modulator and a progestational agent;
a second solid mixture filler/diluent component comprising from about 10% to about 80% by weight of said second solid mixture;
a second solid mixture filler/binder component comprising from about 1% to about 70% by weight of said second solid mixture;
a second solid mixture hydrophilic gel-forming polymer component comprising from about 1% to about 60% of said compressed outer tablet layer;
optionally, a second solid mixture antioxidant component comprising from about 0.01% to about 4% of said second solid mixture; and
optionally, a second solid mixture lubricant component comprising from about 0.01% to about 2% of said second solid mixture.

59. The process of claim 58 further comprising blending said one or more therapeutic agents, said second solid mixture filler/binder component, said second solid mixture filler/diluent component, and said second solid mixture hydrophilic gel-forming polymer component to form said second solid mixture.

60. The process of claim 59 wherein said blending further comprises:
blending said one or more therapeutic agents and said second solid mixture filler/binder component to form an initial mixture; and
blending said initial mixture with said second solid mixture filler/diluent component and said second solid mixture hydrophilic gel-forming polymer component to form said second solid mixture.

61. The process of claim 60 further comprising granulating and then milling said second solid mixture after said blending and prior to said compressing to form said compressed outer tablet layer.

62. The process of claim 61 further comprising blending said second solid mixture antioxidant component and, optionally, at least a portion of said optional second solid mixture lubricant component with said one or more therapeutic agents, said second solid mixture filler/binder component, said second solid mixture filler/diluent component, and said second solid mixture hydrophilic gel-forming polymer component to form said second solid mixture.

63. The process of any one of claims 58 to 62 further comprising blending said first solid mixture filler/diluent component, said first solid mixture filler/binder component, said first solid mixture hydrophilic gel-forming polymer component, and said estrogen to form said first solid mixture.

64. The process of claim 63 further comprising granulating and then milling said first solid mixture after said blending.

65. The process of claim 64 further comprising the steps of:
(a) adding water to said first solid mixture during said granulating; and
(b) drying said first granulated mixture before said milling.

66. The process of claim 65 wherein said drying comprises drying said first granulated mixture to loss on drying (LOD) of from about 1% to about 3%.

67. The process of claim 58 further comprising the steps of:
(i) blending said first solid mixture filler/diluent component, said first solid mixture filler/binder component, said first solid mixture hydrophilic gel-forming polymer component, and said estrogen to form a first solid mixture;
(ii) granulating said first solid mixture of step (i) in the presence of water;
(iiii) drying the first solid mixture of step (ii)
(iv) milling the first solid mixture of step (iii);
(v) optionally, blending said first solid mixture of step (iv) with said optional first solid mixture lubricant component, if present;
(vi) compressing said first solid mixture of step (iv) or step (v), if utilized, to form said core tablet;
(vii) blending said one or more therapeutic agents and said second solid mixture filler/binder component to form an initial mixture;
(viii) blending said initial mixture with said second solid mixture filler/diluent component and said second solid mixture hydrophilic gel-forming polymer component to form a second solid mixture;
(ix) optionally, granulating the second solid mixture of step (viii);
(x) optionally, blending the second solid mixture of step (viii) or step (ix), if utilized, with at least a portion of said optional second solid mixture lubricant component; and
(xi) after step (viii) or steps (ix) or (x), if utilized, compressing the second solid mixture of (vi) onto said core tablet of step (iv) to form said compressed outer tablet layer.

68. The process of any one of claims 58 to 67 wherein:
said first solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said first solid mixture filler/binder component comprises one or more of microcrystalline cellulose, polyvinylpyrrolidone, copovidone, polyvinylalcohol, starch, gelatin, gum arabic, gum acacia, and gum tragacanth;
said first solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional first solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said second solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said second solid mixture filler/binder component comprises one or more of microcrystalline cellulose, polyvinylpyrrolidone, copovidone, polyvinylalcohol, starch, gelatin, gum arabic, gum acacia, and gum tragacanth;
said second solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional second solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional second solid mixture antioxidant component, if present, comprises one or more of ascorbic acid, sodium ascorbate, ascorbyl palmitate, vitamin E, vitamin E acetate, butylated hydroxytoluene, and butylated hydroxyanisole;
said core tablet comprises at least one conjugated estrogen; and
said compressed outer tablet layer comprises medroxyprogesterone acetate or bazedoxifene acetate.

69. The tablet-in-tablet composition of any one of claims 58 to 67 wherein:
said first solid mixture filler/diluent component comprises one or more of lactose and lactose monohydrate;
said first solid mixture filler/binder component comprises microcrystalline cellulose;
said first solid mixture hydrophilic gel-forming polymer component comprises hydroxypropylmethylcellulose;
said optional first solid mixture lubricant component, if present, comprises magnesium stearate;
said second solid mixture filler/diluent component comprises one or more of lactose and lactose monohydrate;
said second solid mixture filler/binder component comprises microcrystalline cellulose;
said second solid mixture hydrophilic gel-forming polymer component comprises hydroxypropylmethylcellulose;
said optional second solid mixture lubricant component, if present, comprises magnesium stearate;
said optional second solid mixture antioxidant component, if present, comprises one or more of ascorbic acid and vitamin E acetate;
said core tablet comprises at least one conjugated estrogen; and
said compressed outer tablet layer comprises medroxyprogesterone acetate or bazedoxifene acetate.

70. The process of any one of claims 58 to 69 wherein said process produces a plurality of tablet-in-tablet compositions having a content uniformity for said therapeutic agent about equal to or less than 3.5%.

71. The process of any one of claims 58 to 69 wherein said process produces a plurality of tablet-in-tablet compositions having a content uniformity for said therapeutic agent about equal to or less than 2.5%.

72. The process of any one of claims 58 to 69 wherein said process produces a plurality of tablet-in-tablet compositions having a weight variation about equal to or less than 2%.

73. The process of any one of claims 58 to 69 wherein said process produces a plurality of tablet-in-tablet compositions having a weight variation about equal to or less than 1.5%.

74. A product of the process of any one of claims 58 to 73.

75. A plurality of products according to claim 74.

76. A product according to claim 74 or claim 75 wherein said compressed outer tablet layer has a hardness from about 2 kp to about 7 kp.

77. A process for producing a tablet-in-tablet composition comprising:
compressing a first solid mixture to form a core tablet; and
compressing a second solid mixture onto said core tablet to form a compressed outer tablet layer;
wherein:
a) said first solid mixture comprises:
one or more estrogens;
a first solid mixture filler/diluent component comprising from about 30% to about 85% by weight by weight of said core tablet;
a first solid mixture filler/binder component comprising from about 1% to about 30% by weight of said core tablet;
a first solid mixture hydrophilic gel-forming polymer component comprising from about 1% to about 40% by weight of said core tablet; and
optionally, a first solid mixture lubricant component comprising from about 0.01% to about 2% by weight of said core tablet; and
b) said second solid mixture comprises:
one or more therapeutic agents selected from the group consisting of selective estrogen receptor modulators and progestational agents;
a pharmaceutically acceptable carrier component comprising from about 60% to about 99.9% by weight of said compressed outer tablet layer, wherein said outer pharmaceutically acceptable carrier component optionally comprises one or more of a second solid mixture filler/diluent component, a second solid mixture filler/binder component, and a second solid mixture hydrophilic gel-forming polymer component;
optionally, a second solid mixture lubricant component comprising from about 0.01% to about 2% by weight of said compressed outer tablet layer; and
optionally, a second solid mixture antioxidant component comprising from about 0.01% to about 4% by weight of said compressed outer tablet layer.

78. The process of claim 77 further comprising blending said one or more therapeutic agents and said pharmaceutically acceptable carrier component to form said second solid mixture.

79. The process of claim 78 further comprising granulating and then milling said second solid mixture prior to compressing to form said compressed outer tablet layer.

80. The process of any one of claims 77 to 79 further comprising blending said first solid mixture filler/diluent component, said first solid mixture filler/binder component, said first solid mixture hydrophilic gel-forming polymer component, and said estrogen to form said first solid mixture.

81. The process of claim 80 further comprising granulating and then milling said first solid mixture prior to said compressing to form said core tablet.

82. The process of claim 81 further comprising the steps of:
(a) adding water to said first solid mixture during said granulating; and
(b) drying said first granulated mixture before said milling.

83. The process of claim 77 further comprising the steps of:
(i) blending said first solid mixture filler/diluent component, said first solid mixture filler/binder component, said first solid mixture hydrophilic gel-forming polymer component, and said estrogen to form a first solid mixture;
(ii) granulating said first solid mixture of step (i) in the presence of water;
(iii) milling said first solid mixture of step (iii) after said granulating;
(iv) optionally, blending said first solid mixture of step (iii) with said optional first solid mixture lubricant component, if present;
(v) compressing said first solid mixture of step (iiii) or optional step (iv), if utilized, to form said core tablet;
(vi) blending said one or more therapeutic agents and said pharmaceutically acceptable carrier component to form an initial mixture;
(vii) optionally, granulating and then milling the second solid mixture of step (vi);
(viii) optionally, blending the second solid mixture of step (vi) or optional step (vii), if utilized, with at least a portion of said optional second solid mixture lubricant component; and
(ix) after step (vi) or optional steps (vi) and (vii), if utilized, compressing the second solid mixture of (vi) onto said core tablet of step (iv) to form said compressed outer tablet layer.

84. The process of any one of claims 77 to 83 wherein:
said first solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said first solid mixture filler/binder component comprises one or more of microcrystalline cellulose, polyvinylpyrrolidone, copovidone, polyvinylalcohol, starch, gelatin, gum arabic, gum acacia, and gum tragacanth;
said first solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said optional first solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said pharmaceutically acceptable carrier component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, a metal carbonate, polyvinylpyrrolidone, copovidone, polyvinylalcohol, gelatin, gum arabic, gum acacia, gum tragacanth, hydroxypropylmethylcellulose, polyethylene oxide, hydroxypropylcellulose, hydroxyethylcellulose, methylcellulose, xanthan gum, and guar gum;
said optional second solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional second solid mixture antioxidant component, if present, comprises one or more of ascorbic acid, sodium ascorbate, ascorbyl palmitate, vitamin E, vitamin E acetate, butylated hydroxytoluene, and butylated hydroxyanisole;
said core tablet comprises at least one conjugated estrogen; and
said compressed outer tablet layer comprises medroxyprogesterone acetate or bazedoxifene acetate.

85. The tablet-in-tablet composition of any one of claims 77 to 83 wherein:
said first solid mixture filler/diluent component comprises one or more of lactose and lactose monohydrate;
said first solid mixture filler/binder component comprises microcrystalline cellulose;
said first solid mixture hydrophilic gel-forming polymer component comprises hydroxypropylmethylcellulose;
said optional first solid mixture lubricant component, if present, comprises magnesium stearate;
said pharmaceutically acceptable carrier component comprises one or more of lactose, lactose monohydrate, microcrystalline cellulose, and hydroxypropylmethylcellulose;
said optional second solid mixture lubricant component, if present, comprises magnesium stearate;
said optional second solid mixture antioxidant component, if present, comprises one or more of ascorbic acid and vitamin E acetate;
said core tablet comprises at least one conjugated estrogen; and
said compressed outer tablet layer comprises medroxyprogesterone acetate or bazedoxifene acetate.

86. The process of any one of claims 77 to 85 wherein said process produces a plurality of tablet-in-tablet compositions having a content uniformity for said therapeutic agent about equal to or less than 3.5%.

87. The process of any one of claims 77 to 85 wherein said process produces a plurality of tablet-in-tablet compositions having a content uniformity for said therapeutic agent about equal to or less than 2.5%.

88. The process of any one of claims 77 to 85 wherein said process produces a plurality of tablet-in-tablet compositions having a weight variation about equal to or less than 2%.

89. The process of any one of claims 77 to 85 wherein said process produces a plurality of tablet-in-tablet compositions having a weight variation about equal to or less than 1.5%.

90. A product of the process of any one of claims 77 to 89.

91. A plurality of products according to claim 90.

92. A product according to claim 90 or claim 91 wherein said compressed outer tablet layer has a hardness from about 2 kp to about 7 kp.

93. A process for producing a tablet-in-tablet composition comprising:
compressing a first solid mixture to form a core tablet; and
compressing a second solid mixture onto said core tablet to form a compressed outer tablet layer;
wherein:
a) said first solid mixture comprises:
one or more estrogens;
a first solid mixture filler/diluent component comprising from about 30% to about 85% by weight by weight of said core tablet;
a first solid mixture filler/binder component comprising from about 1% to about 30% by weight of said core tablet;
a first solid mixture hydrophilic gel-forming polymer component comprising from about 1% to about 40% by weight of said core tablet; and
optionally, a first solid mixture lubricant component comprising from about 0.01% to about 2% by weight of said core tablet; and
b) said second solid mixture comprises:
one or more therapeutic agents selected from the group consisting of selective estrogen receptor modulators and progestational agents;
a second solid mixture filler/diluent component comprising from about 25% to about 65% by weight of said compressed outer tablet layer;
a second solid mixture filler/binder component comprising from about 20% to about 50% by weight of said compressed outer tablet layer;
a second solid mixture disintegrant component comprising from about 2% to about 15% by weight of said compressed outer tablet layer;
optionally, a second solid mixture wetting agent component comprising from about 0.01% to about 4% of said compressed outer tablet layer;
optionally, a second solid mixture lubricant component comprising from about 0.01% to about 2% by weight of said compressed outer tablet layer; and
optionally, a second solid mixture antioxidant component comprising from about 0.01% to about 4% by weight of said compressed outer tablet layer.

94. The process of claim 93 further comprising blending said first solid mixture filler/diluent component, said first solid mixture filler/binder component, said first solid mixture hydrophilic gel-forming polymer component, and said estrogen to form said first solid mixture.

95. The process of claim 94 further comprising granulating and then milling said first solid mixture after said blending.

96. The process of claim 95 further comprising the steps of:
(a) adding water to said first solid mixture during said granulating; and
(b) drying said first granulated mixture before said milling.

97. The process of any one of claims 93 to 96 further comprising blending said one or more therapeutic agents, said optional second solid mixture wetting agent component, if present, and said optional second solid mixture antioxidant component, if present, with at least a portion of each of said second solid mixture filler/diluent component, said second solid mixture filler/binder component, and said second solid mixture disintegrant component to form an initial mixture.

98. The process of claim 97 further comprising granulating and then milling said initial mixture after said blending to form a granulated mixture.

99. The process of claim 98 further comprising blending said granulated mixture with any remaining portion of said second solid mixture filler/diluent component, said second solid mixture filler/binder component and said second solid mixture disintegrant component to form said second solid mixture.

100. The process of claim 99 further comprising blending said second solid mixture with said optional second solid mixture lubricant component, if present, prior to compressing said second solid mixture onto said core tablet.

101. The process of claim 93 further comprising (i) blending said first solid mixture filler/diluent component, said first solid mixture filler/binder component, said first solid mixture hydrophilic gel-forming polymer component, and said estrogen to form a first solid mixture;
(ii) granulating said first solid mixture of step (i) in the presence of water;
(iiii) drying said first solid mixture of step (ii)
(iv) milling said first solid mixture of step (iii);
(v) optionally, blending said first solid mixture of step (iv) with said optional first solid mixture lubricant component, if present;
(vi) compressing said first solid mixture of step (iv) or step (v), if utilized, to form said core tablet;
(vii) blending said one or more therapeutic agents, said optional second solid mixture wetting agent component, if present, and said optional second solid mixture antioxidant component, if present, with at least a portion of each of said second solid mixture filler/diluent component, said second solid mixture filler/binder component, and said second solid mixture disintegrant component to form an initial mixture;
(viii) optionally, granulating and milling said second solid mixture of step
(vii) to form a granulated mixture;
(ix) blending either said initial mixture of (vii) or said granulated mixture of
(viii) with any remaining portion of said second solid mixture filler/diluent component, said second solid mixture filler/binder component and said second solid mixture disintegrant component to form said second solid mixture;
(x) optionally, blending said second solid mixture of step (ix) with at least a portion of said optional second solid mixture lubricant component; and
(xi) compressing said second solid mixture of either step (ix) or step (x) onto said core tablet of step (vi) to form said compressed outer tablet layer.

102. The process of any one of claims 93 to 101 wherein:
said first solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said first solid mixture filler/binder component comprises one or more of microcrystalline cellulose, polyvinylpyrrolidone, copovidone, polyvinylalcohol, starch, gelatin, gum arabic, gum acacia, and gum tragacanth;
said first solid mixture hydrophilic gel-forming polymer component comprises one or more of hydroxypropylmethylcellulose, polyethylene oxide, hydroxypropylcellulose, hydroxyethylcellulose, methylcellulose, polyvinylpyrrolidone, xanthan gum, and guar gum;
said first solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said second solid mixture filler/diluent component comprises one or more of lactose, lactose monohydrate, mannitol, sucrose, maltodextrin, dextrin, maltitol, sorbitol, xylitol, powdered cellulose, cellulose gum, microcrystalline cellulose, starch, calcium phosphate, and a metal carbonate;
said second solid mixture filler/binder component comprises one or more of microcrystalline cellulose, polyvinylpyrrolidone, copovidone, polyvinylalcohol, starch, gelatin, gum arabic, gum acacia, and gum tragacanth;
said second solid mixture disintegrant component comprises one or more of croscarmellose sodium, carmellose calcium, crospovidone, alginic acid, sodium alginate, potassium alginate, calcium alginate, starch, pregelatinized starch, sodium starch glycolate, cellulose floc, and carboxymethylcellulose;
said optional second solid mixture wetting agent component, if present, comprises one or more of a polyethylene glycol-polypropylene glycol copolymer, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid, polyethoxylated fatty acid esters, and polyglycolized glycerides;
said optional second solid mixture lubricant component, if present, comprises one or more of stearic acid, metallic stearate, sodium stearyl fumarate, fatty acid, fatty alcohol, fatty acid ester, glyceryl behenate, mineral oil, vegetable oil, paraffin, leucine, talc, propylene glycol fatty acid ester, polyethylene glycol, polypropylene glycol, and polyalkylene glycol;
said optional second solid mixture antioxidant component, if present, comprises one or more of ascorbic acid, sodium ascorbate, ascorbyl palmitate, vitamin E, vitamin E acetate, butylated hydroxytoluene, and butylated hydroxyanisole;
said core tablet comprises at least one conjugated estrogen; and
said compressed outer tablet layer comprises medroxyprogesterone acetate or bazedoxifene acetate.

103. The process of any one of claims 93 to 101 wherein:
said first solid mixture filler/diluent component comprises one or more of lactose and lactose monohydrate;
said first solid mixture filler/binder component comprises microcrystalline cellulose;
said first solid mixture hydrophilic gel-forming polymer component comprises hydroxypropylmethylcellulose;
said optional first solid mixture lubricant component, if present, comprises magnesium stearate;
said second solid mixture filler/diluent component comprises one or more of lactose and lactose monohydrate;
said second solid mixture filler/binder component comprises microcrystalline cellulose;
said second solid mixture disintegrant component comprises one or more of pregelatinized starch and sodium starch glycolate;
said optional second solid mixture wetting agent component, if present, comprises a polyethylene glycol-polypropylene glycol copolymer;
said optional second solid mixture lubricant component, if present, comprises magnesium stearate;
said optional second solid mixture component, if present, comprises one or more of ascorbic acid and vitamin E acetate;
said core tablet comprises at least one conjugated estrogen; and
said compressed outer tablet layer comprises medroxyprogesterone acetate or bazedoxifene acetate.

104. The process of any one of claims 93 to 103 wherein said process produces a plurality of tablet-in-tablet compositions having a content uniformity for said therapeutic agent about equal to or less than 3.5%.

105. The process of any one of claims 93 to 103 wherein said process produces a plurality of tablet-in-tablet compositions having a content uniformity for said therapeutic agent about equal to or less than 2.5%.

106. The process of any one of claims 93 to 103 wherein said process produces a plurality of tablet-in-tablet compositions having a weight variation about equal to or less than 2%.

107. The process of any one of claims 93 to 103 wherein said process produces a plurality of tablet-in-tablet compositions having a weight variation about equal to or less than 1.5%.

108. A product of the process of any one of claims 93 to 107.

109. A plurality of products according to claim 108.
